SEQUENCE LISTING (2 sequences)

SEQ ID NO: 1 - protein, 371 aa, Homo sapien
  misc_feature: Amino acid sequence for residues 1-359 of the Kinesin motor domain of the human KSP enzyme with a 6-histidine tag at the C-terminus
MGASQPNSSAKKKEEKGKNIQVVVRCRPFNLAERKASAHSIVECDPVRKEVSVRTGGLADKSSRKTYTFDMVFGASTKQIDVYRSVVCPILDEVIMGYNCTIFAYGQTGTGKTFTMEGERSPNEEYTWEEDPLAGIIPRTLHQIFEKLTDNGTEFSVKVSLLEIYNEELFDLLNPSSDVSERLQMFDDPRNKRGVIIKGLEEITVHNKDEVYQILEKGAAKRTTAATLMNAYSSRSHSVFSVTIHMKETTIDGEELVKIGKLNLVDLAGSENIGRSGAVDKRAREAGNINQSLLTLGRVITALVERTPHVPYRESKLTRILQDSLGGRTRTSIIATISPASLNLEETLSTLEYAHRAKNIAMGSSHHHHHH

SEQ ID NO: 2 - DNA, 1116 bp, Homo sapien
  misc_feature: Human cDNA sequence encoding residues 1-359 of the Kinesin motor domain of the human KSP enzyme with a 6-histidine tag at the C-terminus
atgggagcgtcgcagccaaattcgtctgcgaagaagaaagaggagaaggggaagaacatccaggtggtggtgagatgcagaccatttaatttggcagagcggaaagctagcgcccattcaatagtagaatgtgatcctgtacgaaaagaagttagtgtacgaactggaggattggctgacaagagctcaaggaaaacatacacttttgatatggtgtttggagcatctactaaacagattgatgtttaccgaagtgttgtttgtccaattctggatgaagttattatgggctataattgcactatctttgcgtatggccaaactggcactggaaaaacttttacaatggaaggtgaaaggtcacctaatgaagagtatacctgggaagaggatcccttggctggtataattccacgtacccttcatcaaatttttgagaaacttactgataatggtactgaattttcagtcaaagtgtctctgttggagatctataatgaagagctttttgatcttcttaatccatcatctgatgtttctgagagactacagatgtttgatgatccccgtaacaagagaggagtgataattaaaggtttagaagaaattacagtacacaacaaggatgaagtctatcaaattttagaaaagggggcagcaaaaaggacaactgcagctactctgatgaatgcatactctagtcgttcccactcagttttctctgttacaatacatatgaaagaaactacgattgatggagaagagcttgttaaaatcggaaagttgaacttggttgatcttgcaggaagtgaaaacattggccgttctggagctgttgataagagagctcgggaagctggaaatataaatcaatccctgttgactttgggaagggtcattactgcccttgtagaaagaacacctcatgttccttatcgagaatctaaactaactagaatcctccaggattctcttggagggcgtacaagaacatctataattgcaacaatttctcctgcatctctcaatcttgaggaaactctgagtacattggaatatgctcatagagcaaagaacatagccatgggcagcagccatcatcatcatcatcactga

(19) United States
(12) Patent Application Publication
(10) Pub. No.: US 2010/0093767 A1
(43) Pub. Date: Apr. 15, 2010

(54) Mitotic Kinase Inhibitors

(75) Inventors: Jerome C. Bressi, San Diego, CA (US); Andrew John Jennings, San Diego, CA (US); Stephen W. Kaldor, Del Mar, CA (US); Lily Kwok, San Diego, CA (US); Clifford D. Mol, San Diego, CA (US); Jeffrey A. Stafford, San Diego, CA (US)

Correspondence Address: TAKEDA SAN DIEGO, INC., 10410 SCIENCE CENTER DRIVE, SAN DIEGO, CA, 92121, US

(73) Assignee: TAKEDA SAN DIEGO, INC., San Diego, CA (US)

(21) Appl. No.: 11/720,184

(22) PCT Filed: Dec. 2, 2005
(86) PCT No.: PCT/US05/43807

§ 371 Date: Jul. 21, 2008

Related U.S. Application Data
(60) Provisional application No. 60/633,347, filed on Dec. 3, 2004.

(51) Int. Cl. C07D 235/16; A61K 31/4184; A61K 31/4439; C07D 401/12; C07D 471/04; A61K 31/437; A61K 31/52; C07D 473/00; C12N 9/99; A61P 35/04; A61P 9/00; A61P 37/00; A61P 29/00

(52) U.S. Cl. 514/263.1; 548/309.7; 514/394; 514/338; 546/273.4; 548/304.7; 546/118; 514/303; 544/264; 435/184

(57) ABSTRACT

The invention relates to inhibitors of enzymes that disrupt the assembly and function of the mitotic spindle, compositions comprising the inhibitors of Formula (I), kits and articles of manufacture comprising the inhibitors and inhibitor compositions, and methods of using the inhibitors and inhibitor compositions. The inhibitors and inhibitor compositions are useful for treating, preventing or modulating diseases in which mitotic kinesins, including kinesin-like spindle protein (KSP), may be involved; symptoms of such diseases; or the effect of other physiological events mediated by mitotic kinesins, including KSP.

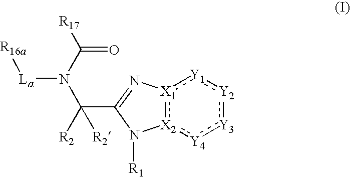

### BACKGROUND OF THE INVENTION

The invention relates to inhibitors of enzymes that disrupt the assembly and function of the mitotic spindle, compositions comprising the inhibitors, kits and articles of manufacture comprising the inhibitors and inhibitor compositions, and methods of using the inhibitors and inhibitor compositions. The inhibitors and inhibitor compositions are useful for treating, preventing or modulating diseases in which mitotic kinesins, including kinesin-like spindle protein (KSP), may be involved; symptoms of such diseases; or the effect of other physiological events mediated by mitotic kinesins, including KSP. The invention also provides for methods of making the inhibitors and methods for treating diseases in which the activities of one or more mitotic kinesins, including KSP, are involved.

Mitotic kinesins are a family of enzymes that regulate the assembly and function of the mitotic spindle, which is responsible for the distribution of replicate copies of the genome to each of the two daughter cells produced during cell division. Specifically, kinesins organize microtubules into a bipolar structure that is the mitotic spindle and mediate movement of chromosomes along spindle microtubules. Accordingly, perturbation of mitotic kinesin function causes malformation or dysfunction of the mitotic spindle, frequently resulting in cell cycle arrest and cell death. Since cancer results, at least in part, from deregulation of the normal processes that control cell division, disruption of the function of the mitotic spindle is expected to retard cancer cell division. Accordingly, the discovery of compounds that inhibit the function of mitotic kinesins, including KSP, is expected to lead to the development of antimitotic chemotherapeutics.

Human KSP (also termed HsEg5; GenBank accession numbers: X85137, NM004523 and U37426) has been described by Blangy et al., Cell, 83:1159-69 (1995); Whitehead et al., Arthritis Rheum., 39:1635-42 (1996); Galgio et al., J. Cell. Biol., 135:339-414 (1996); Blangy et al., J. Biol. Chem., 272:19418-24 (1997); Blangy et al., Cell Motil Cytoskeleton, 40:174-82 (1998); Whitehead and Rattner, J. Cell Sci., 111:2551-61 (1998); and Kaiser et al., JBC, 274:18925-31 (1999). A fragment of the KSP gene (TRIP5; GenBank accession number L40372) has also been reported by Lee et al., Mol. Endocrinol., 9:243-54 (1995). In addition, Xenopus KSP homologs (Eg5) and Drosophila KLP61 F/KRP130 homologs have been reported.

In light of the foregoing, the mitotic kinesins, including but not limited to KSP, are attractive targets for the discovery of new therapeutics due to their important role in mitosis. Specifically, compounds that inhibit mitotic kinesins, including KSP, are expected to lead to the development of therapeutics for the treatment or prevention of proliferative disorders, such as cancer, hyperplasias, restenosis, cardiac hypertrophy, immune disorders, and inflammation, and other diseases.

### SUMMARY OF THE INVENTION

The present invention relates to compounds that have activity for inhibiting mitotic kinesins, including kinesin-like spindle protein (KSP). The present invention also provides compositions, articles of manufacture and kits comprising these compounds.

In one embodiment, a pharmaceutical composition is provided that comprises a mitotic kinesins inhibitor according to the present invention as an active ingredient. Pharmaceutical compositions according to the invention may optionally comprise 0.001%-100% of one or more mitotic kinesin inhibitors of this invention. These pharmaceutical compositions may be administered or coadministered by a wide variety of routes, including for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compositions may also be administered or coadministered in slow release dosage forms.

The invention is also directed to kits and other articles of manufacture for treating disease states associated with mitotic kinesins.

In one embodiment, a kit is provided that comprises a composition comprising at least one mitotic kinesin inhibitor of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one mitotic kinesin inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

Also provided are methods for preparing compounds, compositions and kits according to the present invention. For example, several synthetic schemes are provided herein for synthesizing compounds according to the present invention.

Also provided are methods for using compounds, compositions, kits and articles of manufacture according to the present invention.

In one embodiment, the compounds, compositions, kits and articles of manufacture are used to inhibit mitotic kinesins. In particular, the compounds, compositions, kits and articles of manufacture can be used to inhibit mitotic kinesins.

In another embodiment, the compounds, compositions, kits and articles of manufacture are used to treat a disease state for which mitotic kinesins possess activity that contributes to the pathology and/or symptomology of the disease state.

In another embodiment, a compound is administered to a subject wherein mitotic kinesins activity within the subject is altered, preferably reduced.

In another embodiment, a prodrug of a compound is administered to a subject that is converted to the compound in vivo where it inhibits mitotic kinesins.

In another embodiment, a method of inhibiting mitotic kinesins is provided that comprises contacting a mitotic kinesin with a compound according to the present invention.

In another embodiment, a method of inhibiting mitotic kinesins is provided that comprises causing a compound according to the present invention to be present in a subject in order to inhibit mitotic kinesins in vivo.

In another embodiment, a method of inhibiting a mitotic kinesin is provided that comprises administering a first compound to a subject that is converted in vivo to a second compound wherein the second compound inhibits mitotic kinesin in vivo. It is noted that the compounds of the present invention may be the first or second compounds.

In another embodiment, a therapeutic method is provided that comprises administering a compound according to the present invention.

In another embodiment, a method of treating a condition in a patient which is known to be mediated by mitotic kinesins, or which is known to be treated by mitotic kinesins inhibitors, comprising administering to the patient a therapeutically effective amount of a compound according to the present invention.

In another embodiment, a method is provided for treating a disease state for which mitotic kinesins possess activity that contributes to the pathology and/or symptomology of the disease state, the method comprising: causing a compound according to the present invention to be present in a subject in a therapeutically effective amount for the disease state.

In another embodiment, a method is provided for treating a disease state for which mitotic kinesins possess activity that contributes to the pathology and/or symptomology of the disease state, the method comprising: administering a first compound to a subject that is converted in vivo to a second compound such that the second compound is present in the subject in a therapeutically effective amount for the disease state. It is noted that the compounds of the present invention may be the first or second compounds.

In another embodiment, a method is provided for treating a disease state for which mitotic kinesins possess activity that contributes to the pathology and/or symptomology of the disease state, the method comprising: administering a compound according to the present invention to a subject such that the compound is present in the subject in a therapeutically effective amount for the disease state.

In another embodiment, a method is provided for using a compound according to the present invention in order to manufacture a medicament for use in the treatment of a disease state that is known to be mediated by mitotic kinesins, or that is known to be treated by mitotic kinesins inhibitors.

It is noted in regard to all of the above embodiments that the present invention is intended to encompass all pharmaceutically acceptable ionized forms (e.g., salts) and solvates (e.g., hydrates) of the compounds, regardless of whether such ionized forms and solvates are specified since it is well know in the art to administer pharmaceutical agents in an ionized or solvated form. It is also noted that unless a particular stereochemistry is specified, recitation of a compound is intended to encompass all possible stereoisomers (e.g., enantiomers or diastereomers depending on the number of chiral centers), independent of whether the compound is present as an individual isomer or a mixture of isomers. Further, unless otherwise specified, recitation of a compound is intended to encompass all possible resonance forms and tautomers. With regard to the claims, the language “compound comprising the formula” is intended to encompass the compound and all pharmaceutically acceptable ionized forms and solvates, all possible stereoisomers, and all possible resonance forms and tautomers unless otherwise specifically specified in the particular claim.

It is further noted that prodrugs may also be administered which are altered in vivo and become a compound according to the present invention. The various methods of using the compounds of the present invention are intended, regardless of whether prodrug delivery is specified, to encompass the administration of a prodrug that is converted in vivo to a compound according to the present invention. It is also noted that certain compounds of the present invention may be altered in vivo prior to inhibiting mitotic kinesins, and thus may themselves be prodrugs for another compound. Such prodrugs of another compound may or may not themselves independently have mitotic kinesins inhibitory activity.

### BRIEF DESCRIPTION OF THE FIGURES

FIG. 1 illustrates SEQ. ID Nos. 1 and 2 referred to in this application.

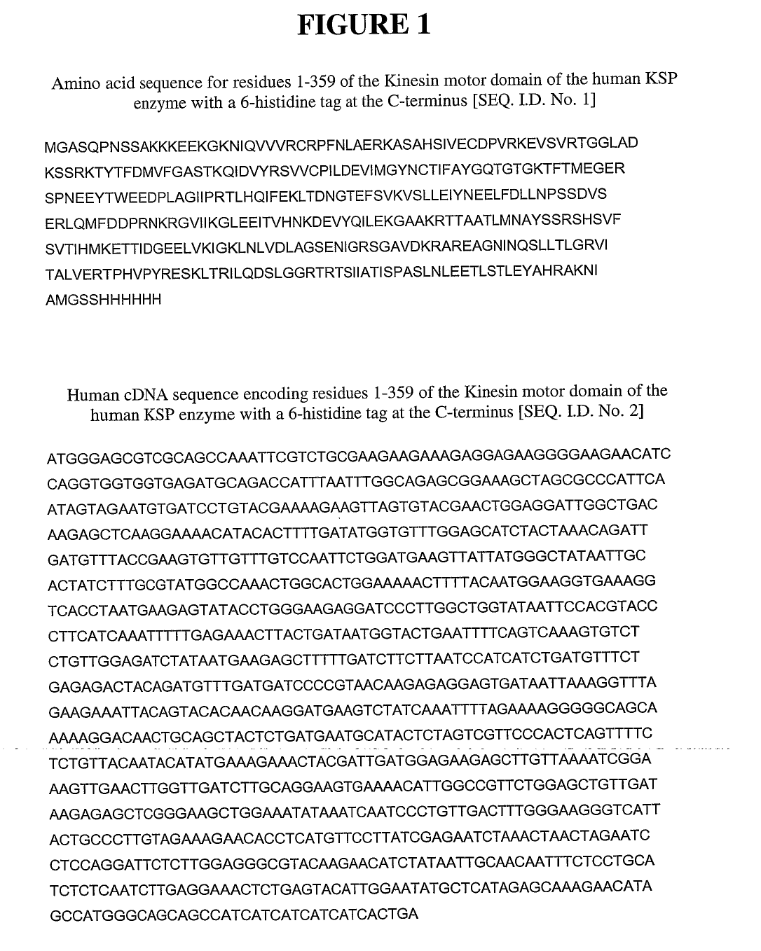

### DEFINITIONS

Unless otherwise stated, the following terms used in the specification and claims shall have the following meanings for the purposes of this Application.

“Alicyclic” means a moiety comprising a non-aromatic ring structure. Alicyclic moieties may be saturated or partially unsaturated with one, two or more double or triple bonds. Alicyclic moieties may also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃₋₈ rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctene, cyclooctene, and cyclooctadiene.

“Aliphatic” means a moiety characterized by a straight or branched chain arrangement of constituent carbon atoms and may be saturated or partially unsaturated with one, two or more double or triple bonds.

“Alkoxy” means an oxygen moiety having a further alkyl substituent. The alkoxy groups of the present invention can be optionally substituted.

“Alkyl” represented by itself means a straight or branched, saturated or unsaturated, aliphatic radical having a chain of carbon atoms, optionally with oxygen (See “oxaalkyl”) or nitrogen atoms (See “aminoalkyl”) between the carbon atoms. C_{X} alkyl and C_{X-Y} alkyl are typically used where X and Y indicate the number of carbon atoms in the chain. For example, C₁₋₆ alkyl includes alkyls that have a chain of between 1 and 6 carbons (e.g., methyl, ethyl, propyl, isopropyl, butyl, sec-butyl, isobutyl, tert-butyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl, heteroarylalkyl) means a straight or branched, saturated or unsaturated aliphatic divalent radical having the number of atoms indicated or when no atoms are indicated means a bond (e.g., (C₆₋₁₀)aryl(C₁₋₃)alkyl includes, benzyl, phenethyl, 1-phenylethyl, 3-phenylpropyl, 2-thienylmethyl, 2-pyridinylmethyl and the like). Where an alkyl moiety forms a component of a chain or a component within a linker, or where the alkyl moiety is substituted, the term “alkyl” is intended to be used synonymously with the term “alkylene.”

“Alkenyl” means a straight or branched, carbon chain that contains at least one carbon-carbon double bond. Examples of alkenyl include vinyl, allyl, isopropenyl, pentenyl, hexenyl, heptenyl, 1-propenyl, 2-butenyl, 2-methyl-2-butenyl, and the like.

“Alkynyl” means a straight or branched, carbon chain that contains at least one carbon-carbon triple bond. Examples of alkynyl include ethynyl, propargyl, 3-methyl-1-pentynyl, 2-heptynyl and the like.

“Alkylene”, unless indicated otherwise, means a straight or branched, saturated or unsaturated, aliphatic, divalent radical. C_{X} alkylene and C_{X-Y} alkylene are typically used where X and Y indicate the number of carbon atoms in the chain. For example, C₁₋₆ alkylene includes methylene (—CH₂—), ethylene(—CH₂CH₂—), trimethylene (—CH₂CH₂CH₂—), tetramethylene (—CH₂CH₂CH₂CH₂—) 2-butenylene (—CH₂CH═CHCH₂—), 2-methyltetramethylene (—CH₂CH(CH₃)CH₂CH₂—), pentamethylene (—CH₂CH₂CH₂CH₂CH₂—) and the like.

“Alkylidene” means a straight or branched saturated or unsaturated, aliphatic radical connected to the parent molecule by a double bond. C_{X} alkylidene and C_{X-Y} alkylidene are typically used where X and Y indicate the number of carbon atoms in the chain. For example, C₁₋₆ alkylidene includes methylene (═CH₂), ethylidene (═CHCH₃), isopropylidene (═C(CH₃)₂), propylidene (═CHCH₂CH₃), allylidene (═CH—CH═CH₂), and the like.

“Amino” means a nitrogen moiety having two further substituents where, for example, a hydrogen or carbon atom is attached to the nitrogen. For example, representative amino groups include —NH₂, —NHCH₃, —N(CH₃)₂, —NHC₁₋₁₀-alkyl, —N(C₁₋₁₀-alkyl)₂, —NHaryl, —NHheteroaryl, —N(aryl)₂, —N(heteroaryl)₂, and the like. Optionally, the two substituents together with the nitrogen may also form a ring. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aminoalkyl” means an alkyl, as defined above, except where one or more substituted or unsubstituted nitrogen atoms (—N—) are positioned between carbon atoms of the alkyl. For example, an (C₂₋₆)aminoalkyl refers to a chain comprising between 2 and 6 carbons and one or more nitrogen atoms positioned between the carbon atoms.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2. An aromatic ring may be such that the ring atoms are only carbon atoms or may include carbon and non-carbon atoms (see Heteroaryl).

“Aryl” means a monocyclic or polycyclic ring assembly wherein each ring is aromatic or when fused with one or more rings forms an aromatic ring assembly. If one or more ring atoms is not carbon (e.g., N, S), the aryl is a heteroaryl. C_{X} aryl and C_{X-Y} aryl are typically used where X and Y indicate the number of atoms in the ring.

“Bicycloalkyl” means a saturated or partially unsaturated fused bicyclic or bridged polycyclic ring assembly.

“Bicycloaryl” means a bicyclic ring assembly wherein the rings are linked by a single bond or fused and at least one of the rings comprising the assembly is aromatic. C_{X} bicycloaryl and C_{X-Y} bicycloaryl are typically used where X and Y indicate the number of carbon atoms in the bicyclic ring assembly and directly attached to the ring.

“Bridging ring” as used herein refers to a ring that is bonded to another ring to form a compound having a bicyclic structure where two ring atoms that are common to both rings are not directly bound to each other. Non-exclusive examples of common compounds having a bridging ring include borneol, norbornane, 7-oxabicyclo[2.2.1]heptane, and the like. One or both rings of the bicyclic system may also comprise heteroatoms.

“Carbamoyl” means the radical —OC(O)NRₐR_{b} where Rₐ and R_{b} are each independently two further substituents where a hydrogen or carbon atom is attached to the nitrogen.

“Carbocycle” means a ring consisting of carbon atoms.

“Carbocyclic ketone derivative” means a carbocyclic derivative wherein the ring contains a —CO— moiety.

“Carbonyl” means the radical —CO—. It is noted that the carbonyl radical may be further substituted with a variety of substituents to form different carbonyl groups including acids, acid halides, aldehydes, amides, esters, and ketones.

“Carboxy” means the radical —CO₂—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Cycloalkyl” means a non-aromatic, saturated or partially unsaturated, monocyclic, fused bicyclic or bridged polycyclic ring assembly. C_{X} cycloalkyl and C_{X-Y} cycloalkyl are typically used where X and Y indicate the number of carbon atoms in the ring assembly. For example, C₃₋₁₀ cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like.

“Cycloalkylene” means a divalent saturated or partially unsaturated, monocyclic or polycyclic ring assembly. C_{X} cycloalkylene and C_{X-Y} cycloalkylene are typically used where X and Y indicate the number of carbon atoms in the ring assembly.

“Disease” specifically includes any unhealthy condition of an animal or part thereof and includes an unhealthy condition that may be caused by, or incident to, medical or veterinary therapy applied to that animal, i.e., the “side effects” of such therapy.

“Fused ring” as used herein refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems may be saturated, partially saturated, carbocyclics, heterocyclics, aromatics, heteroaromatics, and the like.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like (e.g. halo-substituted (C₁₋₃)alkyl includes chloromethyl, dichloromethyl, difluoromethyl, trifluoromethyl, 2,2,2-trifluoroethyl, perfluoroethyl, 2,2,2-trifluoro-1,1-dichloroethyl, and the like).

“Heteroatom” refers to an atom that is not a carbon atom. Particular examples of heteroatoms include, but are not limited to nitrogen, oxygen, and sulfur.

“Heteroatom moiety” includes a moiety where the atom by which the moiety is attached is not a carbon. Examples of heteroatom moieties include —N═, —N_{c}—, N⁺(O⁻)═, —O—, —S— or —S(O)₂—, wherein R_{c} is further substituent.

“Heterobicycloalkyl” means bicycloalkyl, as defined in this Application, provided that one or more of the atoms within the ring is a heteroatom. For example hetero(C₉₋₁₂)bicycloalkyl as used in this application includes, but is not limited to, 3-aza-bicyclo[4.1.0]hept-3-yl, 2-aza-bicyclo[3.1.0]hex-2-yl, 3-aza-bicyclo[3.1.0]hex-3-yl, and the like.

“Heterocycloalkylene” means cycloalkylene, as defined in this Application, provided that one or more of the ring member carbon atoms is replaced by a heteroatom.

“Heteroaryl” means a cyclic aromatic group having five or six ring atoms, wherein at least one ring atom is a heteroatom and the remaining ring atoms are carbon. The nitrogen atoms can be optionally quaternerized and the sulfur atoms can be optionally oxidized. Heteroaryl groups of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrroline, thiazole, 1,3,4-thiadiazole, triazole and tetrazole. “Heteroaryl” also includes, but is not limited to, bicyclic or tricyclic rings, wherein the heteroaryl ring is fused to one or two rings independently selected from the group consisting of an aryl ring, a cycloalkyl ring, a cycloalkenyl ring, and another monocyclic heteroaryl or heterocycloalkyl ring. These bicyclic or tricyclic heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole and 2(1H)-pyridinone. The bicyclic or tricyclic heteroaryl rings can be attached to the parent molecule through either the heteroaryl group itself or the aryl, cycloalkyl, cycloalkenyl or heterocycloalkyl group to which it is fused. The heteroaryl groups of this invention can be substituted or unsubstituted.

“Heterobicycloaryl” means bicycloaryl, as defined in this Application, provided that one or more of the atoms within the ring is a heteroatom. For example, hetero(C₄₋₁₂)bicycloaryl as used in this Application includes, but is not limited to, 2-amino-4-oxo-3,4-dihydropteridin-6-yl, tetrahydroisoquinolinyl, and the like.

“Heterocycloalkyl” means cycloalkyl, as defined in this Application, provided that one or more of the atoms forming the ring is a hetero atom selected, independently from N, O, or S. Non-exclusive examples of heterocycloalkyl include piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

“Hydroxy” means the radical —OH.

“Iminoketone derivative” means a derivative comprising the moiety —C(NR)—, wherein R comprises a hydrogen or carbon atom attached to the nitrogen.

“Isomers” mean any compound having an identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.” A compound that has more than one chiral center has 2ⁿ⁻¹ enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see Advanced Organic Chemistry, 4th edition, March, Jerry, John Wiley & Sons, New York, 1992).

“Nitro” means the radical —NO₂.

“Oxaalkyl” means an alkyl, as defined above, except where one or more oxygen atoms (—O—) are positioned between carbon atoms of the alkyl. For example, an (C₂₋₆)oxaalkyl refers to a chain comprising between 2 and 6 carbons and one or more oxygen atoms positioned between the carbon atoms.

“Oxoalkyl” means an alkyl, further substituted with a carbonyl group. The carbonyl group may be an aldehyde, ketone, ester, amide, acid or acid chloride.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of inhibitors of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Prodrug” means a compound that is convertible in vivo metabolically into an inhibitor according to the present invention. The prodrug itself may or may not also have kinase inhibitory activity. For example, an inhibitor comprising a hydroxy group may be administered as an ester that is converted by hydrolysis in vivo to the hydroxy compound. Suitable esters that may be converted in vivo into hydroxy compounds include acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, an inhibitor comprising an amine group may be administered as an amide that is converted by hydrolysis in vivo to the amine compound.

“Protected derivatives” means derivatives of inhibitors in which a reactive site or sites are blocked with protecting groups. Protected derivatives are useful in the preparation of inhibitors or in themselves may be active as inhibitors. A comprehensive list of suitable protecting groups can be found in T. W. Greene, Protecting Groups in Organic Synthesis, 3rd edition, John Wiley & Sons, Inc. 1999.

“Ring” means a carbocyclic or a heterocyclic system.

“Substituted or unsubstituted” means that a given moiety may consist of only hydrogen substituents through available valencies (unsubstituted) or may further comprise one or more non-hydrogen substituents through available valencies (substituted) that are not otherwise specified by the name of the given moiety. For example, isopropyl is an example of an ethylene moiety that is substituted by —CH₃. In general, a non-hydrogen substituent may be any substituent that may be bound to an atom of the given moiety that is specified to be substituted. Examples of substituents include, but are not limited to, aldehyde, alicyclic, aliphatic, (C₁₋₁₀)alkyl, alkylene, alkylidene, amide, amino, aminoalkyl, aromatic, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl, and oxoalkyl moieties, each of which may optionally also be substituted or unsubstituted.

“Sulfinyl” means the radical —SO—. It is noted that the sulfinyl radical may be further substituted with a variety of substituents to form different sulfinyl groups including sulfinic acids, sulfinamides, sulfinyl esters, and sulfoxides.

“Sulfonyl” means the radical —SO₂—. It is noted that the sulfonyl radical may be further substituted with a variety of substituents to form different sulfonyl groups including sulfonic acids, sulfonamides, sulfonate esters, and sulfones.

“Therapeutically effective amount” means that amount which, when administered to an animal for treating a disease, is sufficient to effect such treatment for the disease.

“Thiocarbonyl” means the radical —CS—. It is noted that the thiocarbonyl radical may be further substituted with a variety of substituents to form different thiocarbonyl groups including thioacids, thioamides, thioesters, and thioketones.

“Treatment” or “treating” means any administration of a compound of the present invention and includes:
(1) preventing the disease from occurring in an animal which may be predisposed to the disease but does not yet experience or display the pathology or symptomatology of the disease,
(2) inhibiting the disease in an animal that is experiencing or displaying the pathology or symptomatology of the diseased (i.e., arresting further development of the pathology and/or symptomatology), or
(3) ameliorating the disease in an animal that is experiencing or displaying the pathology or symptomatology of the diseased (i.e., reversing the pathology and/or symptomatology).

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} may each independently be hydrogen or any other substituent where the atom attached to the carbon is a heteroatom or cyano. Hence, CF₃, CH₂OH and CH₂CN, for example, are all C₁ alkyls.

### DETAILED DESCRIPTION OF THE INVENTION

The present invention relates to compounds, compositions, kits and articles of manufacture that may be used to inhibit mitotic kinesins and, including mitotic kinesin-like spindle protein (IPSP).

### Mitotic Kinesins Inhibitors:

In one embodiment, mitotic kinesins inhibitors of the present invention comprise the formula:

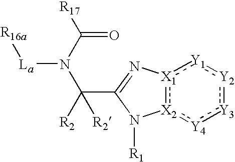

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR₆R₆′, NR₂₃, CO, S, SO, SO₂, and O, with the proviso that R₆′ and R₂₃ are each absent when the atom to which it is bound forms part of a double bond;

Y₂ is selected from the group consisting of CR₇R₇′, NR₂₄, CO, S, SO, SO₂, and O, with the proviso that R₇′ and R₂₄ are each absent when the atom to which it is bound forms part of a double bond;

Y₃ is selected from the group consisting of CR₈R₈′, NR₂₅, CO, S, SO, SO₂, and O, with the proviso that R₈′ and R₂₅ are each absent when the atom to which it is bound forms part of a double bond;

Y₄ is selected from the group consisting of CR₉R₉′, NR₂₆, CO, S, SO, SO₂, and O, with the proviso that R₉′ and R₂₆ are each absent when the atom to which it is bound forms part of a double bond;

R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₆ₐ is an unsubstituted or substituted amino;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

with the provisos that Lₐ is not ethylene when R₁₆ₐ is dimethylamino and Lₐ is not hexyl when R₁₆ₐ is a 2-substituted pyridin-5-yl.

In one aspect of the invention, L is a linker preferably comprising a chain having at least 1 atom. In one variation, the linker comprises at least 1 carbon atom. The linker chain atoms, i.e., the atoms defining the backbone of the linker chain, are selected from the group consisting of C, O, N, and S. In one aspect, the linker chain atoms are selected from the group consisting of C, O, and N, with no oxygen atom being directly bonded to another oxygen atom or to a nitrogen atom of the linker chain or any other nitrogen atom. In another aspect, the linker chain atoms are selected from the group consisting of C and N. In one variation, the linker comprises a chain having about 1 to about 5 chain atoms. In another variation, the linker comprises a chain of 2 to 4 carbon atoms.

In another aspect, there is provided a compound comprising the formula

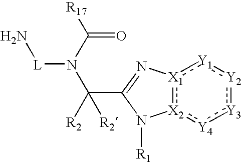

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR₆R₆′, NR₂₃, CO, S, SO, SO₂, and O, with the proviso that R₆′ and R₂₃ are each absent when the atom to which it is bound forms part of a double bond;

Y₂ is selected from the group consisting of CR₇R₇′, NR₂₄, CO, S, SO, SO₂, and O, with the proviso that R₇′ and R₂₄ are each absent when the atom to which it is bound forms part of a double bond;

Y₃ is selected from the group consisting of CR₈R₈′, NR₂₅, CO, S, SO, SO₂, and O, with the proviso that R₈′ and R₂₅ are each absent when the atom to which it is bound forms part of a double bond;

Y₄ is selected from the group consisting of CR₉R₉′, NR₂₆, CO, S, SO, SO₂, and O, with the proviso that R₉′ and R₂₆ are each absent when the atom to which it is bound forms part of a double bond;

R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R₆, R₆′, R₇, R₇′, R₈, R₈¹, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

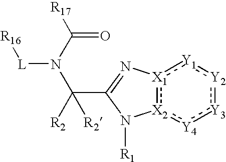

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR₆R₆′, NR₂₃, CO, S, SO, SO₂, and O, with the proviso that R₆′ and R₂₃ are each absent when the atom to which it is bound forms part of a double bond;

Y₂ is selected from the group consisting of CR₇R₇′, NR₂₄, CO, S, SO, SO₂, and O, with the proviso that R₇′ and R₂₄ are each absent when the atom to which it is bound forms part of a double bond;

Y₃ is selected from the group consisting of CR₈R₈′, NR₂₅, CO, S, SO, SO₂, and O, with the proviso that R₈′ and R₂₅ are each absent when the atom to which it is bound forms part of a double bond;

Y₄ is selected from the group consisting of CR₉R₉′, NR₂₆, CO, S, SO, SO₂, and O, with the proviso that R₉′ and R₂₆ are each absent when the atom to which it is bound forms part of a double bond;

R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R₆, R₆′, R₇, R₇′, R₈, R₈¹, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl (C₁₋₅)alkyl, thiocarbonyl(C₁₋₅)alkyl, sulfonyl(C₁₋₃)alkyl, sulfinyl(C₁₋₃)alkyl, imino (C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;

R₁₇ is selected from the group consisting of hydrogen, thio, hydroxy, alkoxy, aryloxy, heteroaryloxy, amino, (C₁₀)alkyl amino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkyl aryl, aryl(C₁₋₆)alkyl, heteroaryl, (C₁₋₆)alkylheteroaryl, and heteroaryl(C₁₋₆)alkyl each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloallcyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl,

with the proviso that R₁₇ is not 3-chloro-benzo[b]thiophene-2-yl when R_{1c} is a 2-piperidinylmethyl-phenyl-methyl.

In another aspect, there is provided a compound comprising the formula

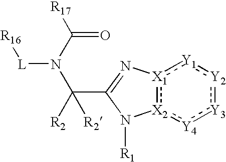

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;

Y₂ is selected from the group consisting of CR_{7d}R_{7d}′, NR₂₄, CO, S, SO, SO₂, and O, with the proviso that R₇′ and R₂₄ are each absent when the atom to which it is bound forms part of a double bond;

Y₃ is selected from the group consisting of CR_{8d}R_{8d}′, NR₂₅, CO, S, SO, SO₂, and O, with the proviso that R₈′ and R₂₅ are each absent when the atom to which it is bound forms part of a double bond;

Y₄ is selected from the group consisting of CR_{9d}R_{9d}′, NR₂₆, CO, S, SO, SO₂, and O, with the proviso that R₉′ and R₂₆ are each absent when the atom to which it is bound forms part of a double bond;

R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R_{6d}, R_{6d}′, R_{7d}, R_{7d}′, R_{8d}, R_{8d}′, R_{9d} and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl (C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;

R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloallcyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

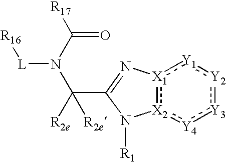

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR₆R₆′, NR₂₃, CO, S, SO, SO₂, and O, with the proviso that R₆′ and R₂₃ are each absent when the atom to which it is bound forms part of a double bond;

Y₂ is selected from the group consisting of CR₇R₇′, NR₂₄, CO, S, SO, SO₂, and O, with the proviso that R₇′ and R₂₄ are each absent when the atom to which it is bound forms part of a double bond;

Y₃ is selected from the group consisting of CR₈R₈′, NR₂₅, CO, S, SO, SO₂, and O, with the proviso that R₈′ and R₂₅ are each absent when the atom to which it is bound forms part of a double bond;

Y₄ is selected from the group consisting of CR₉R₉′, NR₂₆, CO, S, SO, SO₂, and O, with the proviso that R₉′ and R₂₆ are each absent when the atom to which it is bound forms part of a double bond;

R₁ is hydrogen, or is selected from the group consisting of (C₁₋₆)alkyl, (C₁₋₆)alkyloxy(C₁₋₆)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;

R₂′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂′ are taken together to form a 3- to 7-membered ring, each substituted or unsubstituted;

R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl (C₁₋₅)alkyl, thiocarbonyl(C₁₋₅)alkyl, sulfonyl(C₁₋₃)alkyl, sulfinyl(C₁₋₃)alkyl, imino (C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₄₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

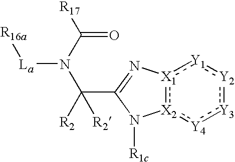

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR₆R₆′, NR₂₃, CO, S, SO, SO₂, and O, with the proviso that R₆′ and R₂₃ are each absent when the atom to which it is bound forms part of a double bond;

Y₂ is selected from the group consisting of CR₇R₇′, NR₂₄, CO, S, SO, SO₂, and O, with the proviso that R₇′ and R₂₄ are each absent when the atom to which it is bound forms part of a double bond;

Y₃ is selected from the group consisting of CR₈R₈′, NR₂₅, CO, S, SO, SO₂, and O, with the proviso that R₈′ and R₂₅ are each absent when the atom to which it is bound forms part of a double bond;

Y₄ is selected from the group consisting of CR₉R₉′, NR₂₆, CO, S, SO, SO₂, and O, with the proviso that R₉′ and R₂₆ are each absent when the atom to which it is bound forms part of a double bond;

R_{1c} is selected from the group consisting of (C₁₋₆)alkyl(C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₆ₐ is an unsubstituted or substituted amino;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicyclo hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

In another aspect, there is provided a compound comprising the formula

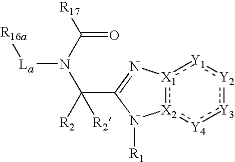

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;

Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;

Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;

Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;

R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)cycloalkyl, hetero(C₃₋₁₂)cycloalkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;

R₁₆ₐ is an unsubstituted or substituted amino;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

In another aspect, there is provided a compound comprising the formula

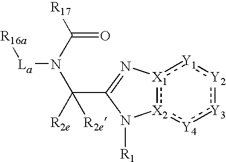

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR₆R₆′, NR₂₃, CO, S, SO, SO₂, and O, with the proviso that R₆′ and R₂₃ are each absent when the atom to which it is bound forms part of a double bond;

Y₂ is selected from the group consisting of CR₇R₇′, NR₂₄, CO, S, SO, SO₂, and O, with the proviso that R₇′ and R₂₄ are each absent when the atom to which it is bound forms part of a double bond;

Y₃ is selected from the group consisting of CR₈R₈′, NR₂₅, CO, S, SO, SO₂, and O, with the proviso that R₈′ and R₂₅ are each absent when the atom to which it is bound forms part of a double bond;

Y₄ is selected from the group consisting of CR₉R₉′, NR₂₆, CO, S, SO, SO₂, and O, with the proviso that R₉′ and R₂₆ are each absent when the atom to which it is bound forms part of a double bond;

R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;

R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;

R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₆ₐ is an unsubstituted or substituted amino;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

In another aspect, there is provided a compound comprising the formula

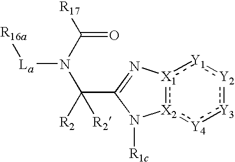

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;

Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;

Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;

Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;

R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl (C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;

R₁₆ₐ is an unsubstituted or substituted amino;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo((C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

In another aspect, there is provided a compound comprising the formula

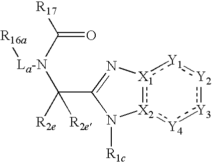

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR₆R₆′, NR₂₃, CO, S, SO, SO₂, and O, with the proviso that R₆′ and R₂₃ are each absent when the atom to which it is bound fowls part of a double bond;

Y₂ is selected from the group consisting of CR₇R₇′, NR₂₄, CO, S, SO, SO₂, and O, with the proviso that R₇′ and R₂₄ are each absent when the atom to which it is bound forms part of a double bond;

Y₃ is selected from the group consisting of CR₈R₈′, NR₂₅, CO, S, SO, SO₂, and O, with the proviso that R₈′ and R₂₅ are each absent when the atom to which it is bound forms part of a double bond;

Y₄ is selected from the group consisting of CR₉R₉′, NR₂₆, CO, S, SO, SO₂, and O, with the proviso that R₉′ and R₂₆ are each absent when the atom to which it is bound forms part of a double bond;

R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

R₂ₑ is selected from the group consisting of (C₁₋₆)alkyl, (C₁₋₃)alkyloxy(C₁₋₃)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;

R₂ₑ′ is selected from the group consisting of hydrogen, (C₁₋₆)alkyl, (C₁₋₃)alkyloxy(C₁₋₃)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;

R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₆ₐ is an unsubstituted or substituted amino;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

In another aspect, there is provided a compound comprising the formula

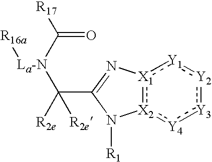

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;

Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;

Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;

Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;

R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;

R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;

R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl (C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;

R₁₆ₐ is an unsubstituted or substituted amino;

R₁₇ is selected from the group consisting of hydrogen, thio, hydroxy, alkoxy, aryloxy, heteroaryloxy, amino, (C₁₋₁₀)alkylarnino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

In another aspect, there is provided a compound comprising the formula

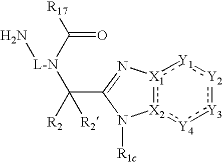

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR₆R₆′, NR₂₃, CO, S, SO, SO₂, and O, with the proviso that R₆′ and R₂₃ are each absent when the atom to which it is bound forms part of a double bond;

Y₂ is selected from the group consisting of CR₇R₇′, NR₂₄, CO, S, SO, SO₂, and O, with the proviso that R₇′ and R₂₄ are each absent when the atom to which it is bound forms part of a double bond;

Y₃ is selected from the group consisting of CR₈R₈′, NR₂₅, CO, S, SO, SO₂, and O, with the proviso that R₈′ and R₂₅ are each absent when the atom to which it is bound forms part of a double bond;

Y₄ is selected from the group consisting of CR₉R₉′, NR₂₆, CO, S, SO, SO₂, and O, with the proviso that R₉′ and R₂₆ are each absent when the atom to which it is bound forms part of a double bond;

R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloallcyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₃ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkyl aryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

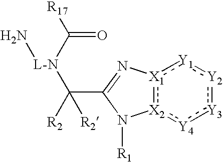

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;

Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;

Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;

Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;

R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl (C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;

R₁₇ is selected from the group consisting of hydrogen, thio, hydroxy, alkoxy, aryloxy, heteroaryloxy, amino, (C₁₋₁₀)alkyl amino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

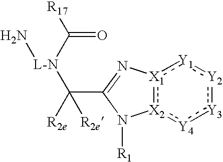

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR₆R₆′, NR₂₃, CO, S, SO, SO₂, and O, with the proviso that R₆′ and R₂₃ are each absent when the atom to which it is bound foul's part of a double bond;

Y₂ is selected from the group consisting of CR₇R₇′, NR₂₄, CO, S, SO, SO₂, and O, with the proviso that R₇′ and R₂₄ are each absent when the atom to which it is bound forms part of a double bond;

Y₃ is selected from the group consisting of CR₈R₈′, NR₂₅, CO, S, SO, SO₂, and O, with the proviso that R₈′ and R₂₅ are each absent when the atom to which it is bound forms part of a double bond;

Y₄ is selected from the group consisting of CR₉R₉′, NR₂₆, CO, S, SO, SO₂, and O, with the proviso that R₉′ and R₂₆ are each absent when the atom to which it is bound forms part of a double bond;

R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;

R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;

R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkyl aryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

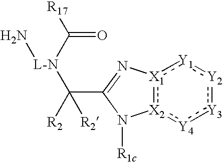

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;

Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;

Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;

Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;

R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R_{6d}, R_{6d}′, R_{7d}, R_{7d}′, R_{8d}, R₈′, R_{9d}, and R_{9d}′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)allcylhetero(C₃₋₇)cycloalkyl, hetero(C₃₋₇)cyclo alkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

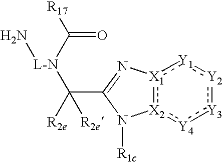

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR₆R₆′, NR₂₃, CO, S, SO, SO₂, and O, with the proviso that R₆′ and R₂₃ are each absent when the atom to which it is bound forms part of a double bond;

Y₂ is selected from the group consisting of CR₇R₇′, NR₂₄, CO, S, SO, SO₂, and O, with the proviso that R₇′ and R₂₄ are each absent when the atom to which it is bound forms part of a double bond;

Y₃ is selected from the group consisting of CR₈R₈′, NR₂₅, CO, S, SO, SO₂, and O, with the proviso that R₈′ and R₂₅ are each absent when the atom to which it is bound forms part of a double bond;

Y₄ is selected from the group consisting of CR₉R₉′, NR₂₆, CO, S, SO, SO₂, and O, with the proviso that R₉′ and R₂₆ are each absent when the atom to which it is bound forms part of a double bond;

R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;

R₂′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂′ are taken together to form a 3- to 7-membered ring, each substituted or unsubstituted;

R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₇ is selected from the group consisting of hydrogen, thio, hydroxy, alkoxy, aryloxy, heteroaryloxy, amino, (C₁₋₁₀)alkyl amino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

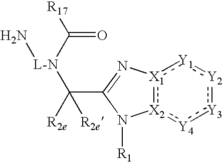

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR_{6d}R_{6d}′, NR₂₃, CO, S, SO, SO₂, and O, with the proviso that Rd and R₂₃ are each absent when the atom to which it is bound forms part of a double bond;

Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;

Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;

Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;

R₁ is hydrogen, or is selected from the group consisting of (C₁₋₆)alkyl, (C₁₋₆)alkyloxy(C₁₋₆)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;

R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;

R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl (C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

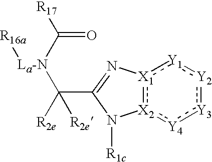

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;

Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;

Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;

Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;

R_{1c} is selected from the group consisting of (C₁₋₆)alkyl, (C₁₋₃)alkyloxy(C₁₋₃)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;

R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;

R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl (C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;

R₁₆ₐ is an unsubstituted or substituted amino;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylarnino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

In another aspect, there is provided a compound comprising the formula

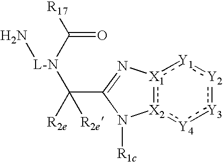

wherein:

X₁ and X₂ are each independently selected from the group consisting of CR₁₈ and N, with the proviso that R₁₈ is absent when the carbon to which it is bound forms part of a double bond;

Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;

Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;

Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;

Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;

R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

R₃ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;

R₂ₑ′ is selected from the group consisting of hydrogen, (C₁₋₆)alkyl, (C₁₋₃)alkyloxy(C₁₋₃)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;

R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl (C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;

R₁₋₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;

R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

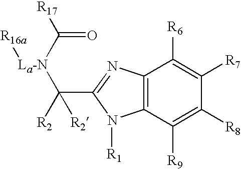

wherein:

R₁ is hydrogen, or is selected from the group consisting of (C₁₋₆)alkyl, (C₁₋₆)alkyloxy(C₁₋₆)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R₆, R₇, R₈, and R₉ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₆ₐ is an unsubstituted or substituted amino;

R₁₇ is selected from the group consisting of hydrogen, thio, hydroxy, alkoxy, aryloxy, heteroaryloxy, amino, (C₁₋₁₀)alkyl amino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

with the provisos that Lₐ is not ethyl when R₁₆ₐ is dimethylamino and Lₐ is not hexyl when R₁₆ₐ is a 2-substituted pyridine-5-yl.

In one aspect of the invention, L is a linker preferably comprising a chain having at least 1 atom. In one variation, the linker comprises at least 1 carbon atom. The linker chain atoms, i.e., the atoms defining the backbone of the linker chain, are selected from the group consisting of C, O, N, S, and Si. In one aspect, the linker chain atoms are selected from the group consisting of C, O, and N, with no oxygen atom being directly bonded to another oxygen atom or to a nitrogen atom of the linker chain or any other nitrogen atom. In another aspect, the linker chain atoms are selected from the group consisting of C and N. In one variation, the linker comprises a chain having about 1 to about 5 chain atoms. In another variation, the linker comprises a chain of 2 to 4 carbon atoms.

In another aspect, there is provided a compound comprising the formula

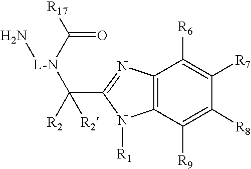

wherein:

R₁ is hydrogen, or is selected from the group consisting of (C₁₋₆)alkyl, (C₁₋₆)alkyloxy(C₁₋₆)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R₆, R₇, R₈ and R₉ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₇ is selected from the group consisting of hydrogen, thio, hydroxy, alkoxy, aryloxy, heteroaryloxy, amino, (C₁₋₁₀)alkyl amino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkyl aryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

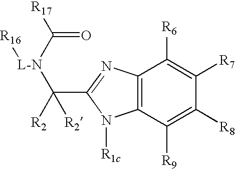

wherein:

R₁ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R₆, R₇, R₈, and R₉ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl (C₁₋₅)alkyl, thiocarbonyl(C₁₋₅)alkyl, sulfonyl(C₁₋₃)alkyl, sulfinyl(C₁₋₃)alkyl, imino (C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

with the proviso that R₁₇ is not 3-chloro-benzo[b]thiophene-2-yl when R₁ is a 2-piperidinylmethyl-phenyl-methyl.

In another aspect, there is provided a compound comprising the formula

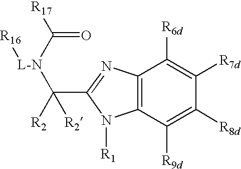

wherein:

R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R_{6d}, R_{7d}, R_{8d} and R_{9d} are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl(C₁₋₅)alkyl, sulfonyl (C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;

R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl (C₁₋₅)alkyl, thiocarbonyl(C₁₋₅)alkyl, sulfonyl(C₁₋₃)alkyl, sulfinyl(C₁₋₃)alkyl, imino (C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;

R₁₇ is selected from the group consisting of hydrogen, thio, hydroxy, alkoxy, aryloxy, heteroaryloxy, amino, (C₁₋₁₀)alkylamino, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another aspect, there is provided a compound comprising the formula

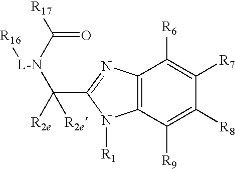

wherein:

R₁ is hydrogen, or is selected from the group consisting of (C₁₋₆)alkyl, (C₁₋₆)alkyloxy(C₁₋₆)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;

R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;

R₆, R₇, R₈, and R₉ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl (C₁₋₅)alkyl, thiocarbonyl(C₁₋₅)alkyl, sulfonyl(C₁₋₃)alkyl, sulfinyl(C₁₋₃)alkyl, imino (C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;

R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

### Substituents X₁, X₂, Y₁, Y₂, Y₃, and Y₄:

In one variation of the above compound, X₁ and X₂ are both C and form part of a double bond. In another variation of the above, Y₁ is CR₆ and forms part of a double bond. In yet another variation, Y₂ is CR₇ and forms part of a double bond. In yet another variation, Y₃ is CR₈ and forms part of a double bond. In another variation, Y₄ is CR₉ and forms part of a double bond.

### Substituent R₁:

In one variation of the above compound, R₁ is selected from the group consisting of (C₁₋₆)alkyl, aryl(C₁₋₆)alkyl, and heteroaryl(C₁₋₆)alkyl, each substituted or unsubstituted. In another variation, R₁ is selected from the group consisting of methyl, benzyl, thiophene-yl-methyl, and pyridinylmethyl, each substituted or unsubstituted. In yet another variation of the above, R_{1c} is selected from the group consisting of (C₁₋₆)alkyl, (C₁₋₆)alkyloxy(C₁₋₆)alkyl, aryl, (C₁₋₆)alkylaryl, heteroaryl, (C₁₋₆)alkylheteroaryl, and hetero(C₃₋₇)cycloalkyl, each substituted or unsubstituted.

In a particular variation of the above, R₁ is selected from the group consisting of (C₁₋₆)alkyl, aryl, (C₁₋₆)alkyl aryl, heteroaryl, (C₁₋₆)alkylheteroaryl, and hetero(C₃₋₇)cycloalkyl, each substituted or unsubstituted. In another particular variation, R₁ is selected from the group consisting of benzyl, phenyl, methylthiophene-yl, and methylfuranyl, each substituted or unsubstituted. In yet another variation, R₁ is selected from the group consisting of methyl, ethyl, propyl, methoxymethyl, methoxyethyl, ethoxymethyl, ethoxyethyl, phenyl, 4-morpholinyl-(C₂₋₆)alkenyl, 4-morpholin-4-yl-buten-2-yl, pyrrolidin-1-yl-(C₂₋₆)alkenyl, 2-, 3-, or 4-halophenyl, 2-, 3- or 4-(C₁₋₃)alkylphenyl, 2,4-di-(C₁₋₃)alkylphenyl, 3,4-di-(C₁₋₃)alkylphenyl, 3- or 4-(C₁₋₃)alkoxyphenyl, 2-(C₁₋₃)alkyl-3-halophenyl, 2,4-di-halophenyl, 3,4-di-halophenyl, 2-(C₁₋₃)alkyl-4-halophenyl, 2-, 3- or 4-(C₁₋₃)alkylbenzyl, 2,4-di-(C₁₋₃)alkylbenzyl, 3,4-di-(C₁₋₃)alkylbenzyl, 3 or 4-(C₁₋₃)alkoxybenzyl, 2-, 3- or 4-halobenzyl, 2-(C₁₋₃)alkyl-3-halobenzyl, 2,4-di-halobenzyl, 3,4-di-halobenzyl, 2-(C₁₋₃)alkyl-4-halobenzyl, and (C₁₋₅)alkoxycarbonyl-(C₁₋₆)alkyl, each optionally further substituted.

In one variation of the above compound, R₁ is selected from the group consisting of naphthyl, naphthalen(C₁₋₃)alkyl, 2-tetrahydrofuran-methyl-, piperidinyl, piperidino(C₁₋₃)alkyl, 2-imidazol-1-ylmethyl-benzyl, 4-morpholinyl, 4-morpholino(C₁₋₆)alkyl 4-piperazinyl, 2-piperidin-1-ylmethyl-benzyl, 2-(3,3-dimethyl-piperidin-1-ylmethyl)-benzyl, 2-diethylaminomethyl-benzyl, 2-((ethyl-methyl-amino)methyl)-benzyl, 2-(4-methyl-piperidin-1-ylmethyl)-benzyl, (2-morpholin-4-yl-methylbenzyl), 2-(4-methyl-piperazin-1-ylmethyl)-benzyl, 4-piperazino(C₁₋₃)alkyl, pyrrolidinyl, pyrrolidino(C₁₋₃)alkyl, perhydropyrrolizinyl, perhydropyrrolizino(C₁₋₃)alkyl, 1,4-diazaperhydroepinyl, 1,4-diazaperhydroepinyl(C₁₋₃)alkyl, 1,3-dioxanyl, 1,4-dioxanyl(C₁₋₃)alkyl, 1,3-dioxanyl, and 1,4-dioxanyl(C₁₋₃)alkyl, each substituted or unsubstituted.

Substituents R₂ and R₂′:

According to each of the above variations, there is provided a compound wherein R₂ is selected from the group consisting of hydrogen and (C₁₋₆)alkyl, each substituted or unsubstituted. In another variation of the above, R₂ is selected from the group consisting of methyl, ethyl and propyl, each substituted or unsubstituted. According to each of the above variation, R₂′ is hydrogen.

According to each of the above variations, R₂ and R₂′ are each independently hydrogen or are each independently selected from the group consisting of (C₁₋₆)alkyl, (C₁₋₃)alkyloxy(C₁₋₃)alkyl, aryl, (C₁₋₁₀)alkylaryl, heteroaryl, (C₃₋₁₂)cycloalkyl, hetero(C₃₋₁₂)cycloalkyl, (C₁₋₆)alkylheteroaryl, and the side chain of an amino acid, or R₂ and R₂′ are taken together to form a 3- to 7-membered ring, each substituted or unsubstituted. In yet another variation, R₂ and R₂′ are each independently hydrogen or the side chain of an amino acid.

The amino acids may either be naturally occurring or non-naturally occurring. The side chain of the amino acid above may be in either the R or the S configuration. In a preferred aspect of the invention, the amino acids are in the R or D-configuration. In a preferred aspect, the functional groups above that corresponds to the “side chains of the amino acid” are in the R configuration when the side chains are incorporated in the compound above.

According to the above variation, R₂ and R₂′ are taken together to form a 3- to 7-membered ring, each substituted or unsubstituted. In another variation, R₂ and R₂′ are each independently hydrogen or are each selected from the group consisting of an unsubstituted or substituted (R) or (S) (C₁₋₅)alkyl. In yet another variation, R₂ and R₂′ are each independently hydrogen or are each selected from the group consisting of an unsubstituted or substituted (R) (C₁₋₅)alkyl.

According to each of the above variations, there is provided a compound wherein the stereogenic center to which R₂ and R₂′ are attached is in the R configuration.

### Substituent R₂ₑ and R₂ₑ′:

According to particular variation of the above, R₂ₑ is selected from the group consisting of (C₁₋₁₀)alkyl, (C₁₋₆)alkyloxy(C₁₋₆)alkyl, aryl(C₁₋₁₀)alkyl, aryl, (C₁₋₁₀)alkylaryl, heteroaryl, heteroaryl(C₁₋₆)alkyl, hetero(C₃₋₁₂)cycloalkyl, (C₁₋₆)alkylheteroaryl, and the side chain of an amino acid. In one variation, R₂ₑ′ is selected from the group consisting of hydrogen, (C₁₋₁₀)alkyl, (C₁₋₆)alkyloxy(C₁₋₆)alkyl, aryl(C₁₋₁₀)alkyl, aryl, (C₁₋₁₀)alkylaryl, heteroaryl, heteroaryl(C₁₋₆)alkyl, hetero(C₃₋₁₂)cycloalkyl, (C₁₋₆)alkylheteroaryl, and the side chain of an amino acid, or R₂ₑ and R₂ₑ′ are taken together to form a 3- to 7-membered ring, each substituted or unsubstituted.

### Substituent R₆, R₇, R₈, and R₉:

According to particular variations of the above, there is provided a compound wherein R₆, R₇, R₈, and R₉ are each independently selected from the group consisting of hydrogen, halo, a substituted or unsubstituted (C₁₋₆)alkyl, and cyano. In one variation, R₆, R₇, R₈, R₉ are each independently hydrogen or are each selected from the group consisting of halo, halo(C₁₋₆)alkyl, amino, nitro, cyano, thio, (C₁₋₆)alkyl, cycloalkyl, heterocycloalkyl, aryl, heteroaryl, carbonyl(C₁₋₅)alkyl, thiocarbonyl(C₁₋₅)alkyl, sulfonyl(C₁₋₃)alkyl, sulfinyl (C₁₋₃)alkyl, imino(C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring.

In another variation, R₆, R₇, R₈, R₉ are each independently hydrogen or are each independently selected from the group consisting of (C₁₋₆)dialkylamino, (C₁₋₆)alkylsulfonyl, (C₁₋₆)alkylsulfonamido, sulfonamido(C₁₋₆)alkyl, sulfonamidoaryl, (C₁₋₆)alkylthio, carboxy(C₁₋₆)alkyl, carboxamido, aminocarbonyl, aryl and heteroaryl, each substituted or unsubstituted. In yet another variation of the above, R₆, R₇, R₈, R₉ are each independently hydrogen or are each independently selected from the group consisting of —C(O)NH₂, —SO₂NH₂, —COOH, (C₁₋₆)alkyl, (C₂₋₆)alkenyl, (C₂₋₆)alkenyl, (C₁₋₆)alkoxy, (C₁₋₆)alkoxyCO—, (C₁₋₆)alkylCO—, (C₁₋₆)alkylthio-, (C₁₋₆)alkylNH—, ((C₁₋₆)alkyl)₂N—, (C₁₋₆)alkylOCONH—, (C₁₋₆)alkylSO₂NH—, (C₁₋₆)alkylSONH—, and (C₁₋₆)alkylOCONH—, each unsubstituted or further substituted with the group selected from hydroxy, halogen, amino, cyano, nitro, (C₁₋₆)alkyl and halo(C₁₋₆)alkyl.

According to particular variation of the above, there is provided a compound wherein R₆, R₇, R₈, R₉ are each independently selected from the group consisting of fluoro, chloro, bromo, iodo, cyano, and nitro. In one variation, R₇ and R₈ are each independently selected from the group consisting of fluoro, chloro, bromo, iodo, cyano, and nitro. In another variation, R₈ and R₉ are each H. In yet another variation, R₇ and R₈ are each independently selected from the group consisting of halo, (C₁₋₆)alkyl, halo(C₁₋₆)alkyl, and (C₁₋₆)alkoxy.

Substituent R₁₆:

According to one variation of the above, R₁₆ is a substituted or unsubstituted amino. In another particular variation, R₁₆ is NH₂. In yet another variation, R₁₆ is selected from the group consisting of 3-chloro-benzo[b]thiophene, 2,3-difluorophenyl, 4-methylthiophenyl, 2-ethoxyphenyl, 2-chloro-5-trifluoromethylphenyl, 1-naphthyl, 8-bromo-1-naphthyl, 3-fluoro-4-methoxyphenyl, 2-methyl-5-fluorophenyl, 4-chlorophenyl, 2,5-dichlorophenyl and 2-chlorothienyl, each further substituted or unsubstituted. In another variation of the above, there is provided a compound wherein R₁₆ is selected from the group consisting of phenyl, —CH═CH-phenyl, phenylcyclopropyl-, pyridinyl, quinolinyl, indolinyl, quinazolinyl, isothiazolyl, 1H-pyrazolyl, and thiophenyl, each substituted or unsubstituted.

In one variation of the above, R₁₆ is selected from the group consisting of methyl, i-propyl, t-butyl, MeCO₂—, —CO₂H, cyclohexyl, hydroxy, methoxy, —NH₂, —NHMe, —NMe₂, —NEt₂, —NH(i-propyl), —N(i-propyl)₂, —NHBoc, phenyl, 2-methyl-, 2-ethyl- or 2-methoxy-phenyl, 3-methyl-, 3-ethyl-, or 3-hydroxyphenyl, and 2,5-dimethyl or 2,5-dimethoxyphenyl, each substituted or unsubstituted. In another variation, R₁₆ is selected from the group consisting of pyrrolidin-1-yl, pyrrolidin-2-yl, N-methyl or N-ethylpyrrolidin-2-yl, pyrrolidin-2-one-1-yl, piperazin-1-yl, morpholin-1-yl, 3-pyridyl, 1H-indol-1-yl, 1H-imidazol-4-yl, 1H-imidazol-1-yl, tetrahydrofuran-2-yl-methyl, azetidin-1-yl, azetidin-2-yl, azetidin-3-yl, piperidin-1-yl, 2-methylpiperidin-1-yl, piperidin-4-yl, piperidin-3-yl, piperidin-2-yl, and N-methylpiperidin-1-yl, each unsubstituted or further substituted.

According to one variation of the above, there is provided a compound wherein -L-R₁₆ together is selected from the group consisting of 3-dimethylamino-2,2-di(C₁₋₃)alkylpropanyl, 4-di(C₁₋₃)alkylamino-pentan-2-yl, 4-di(C₁₋₃)alkylamino-1-cyclohexyl-pentanyl, 4-di(C₁₋₃)alkylamino-ethyl, and N-benzyl-piperidin-4-yl, each unsubstituted or further substituted.

### Substituent R₁₇:

According to each of the above variations, there is provided a compound wherein R₁₇ is selected from the group consisting of aryl(C₁₋₆)alkyl, (C₃₋₇)cycloalkyl, hetero(C₃₋₇)cycloalkyl, haloaryl, (C₁₋₆)alkylaryl, (C₁₋₆)alkoxyaryl, cyanoaryl, heteroaryl, haloheteroaryl, (C₁₋₆)alkylheteroaryl, (C₁₋₆)alkyl-thio-heteroaryl, and heterobicycloaryl, each substituted or unsubstituted.

According to each of the above variations, there is provided a compound wherein R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, halo(C₁₋₁₀)alkyl, (C₃₋₁₂)cycloalkyl(C₁₋₁₀)alkyl, amino(C₁₋₁₀)alkyl, aryl, heteroaryl, (C₉₋₁₂)bicycloaryl, and hetero(C₄₋₁₂)bicycloaryl, each substituted or unsubstituted.

According to each of the above variations, R₁₇ is hydrogen or is selected from the group consisting of halo(C₁₋₆)alkyl, amino, (C₁₋₁₅)alkyl, cycloalkyl, hetero(C₃₋₁₂)cycloalkyl, aryl, heteroaryl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, aryloxy, heteroaryloxy, each substituted or unsubstituted, and a substituted or unsubstituted 4, 5, 6 or 7 membered ring.

According to another variation of the above, there is provided a compound wherein R₁₇ is hydrogen or is selected from the group consisting of (C₁₋₁₅)alkyl, aryl, aryl(C₁₋₃)alkyl, heteroaryl, heteroaryl(C₁₋₃)alkyl, each substituted or unsubstituted, R₁₄O(C₁₋₆)alkyl where R₁₄ is H or is selected from the group consisting of (C₁₋₃)alkyl, aryl, aryl(C₁₋₃)alkyl, heteroaryl, heteroaryl(C₁₋₃)alkyl, each substituted or unsubstituted, (C₁₋₃)alkoxyCO(C₁₋₃)alkyl, [(C₁₋₃)alkoxy(C₁₋₃)alkyl]¹⁻³, aryl[(C₁₋₃)alkoxy(C₁₋₃)alkyl]₁₋₃, [(C₁₋₃)alkoxy(C₁₋₃)alkyl]₁₋₃aryl, and [(C₁₋₃)alkoxy(C₁₋₃)alkyl]₁₋₃aryl, each substituted or unsubstituted, or R₁₄R₁₅N(C₁₋₆)alkyl where R₁₄ and R₁₅ are each independently H or (C₁₋₃)alkyl, aryl, aryl(C₁₋₃)alkyl, heteroaryl, and heteroaryl(C₁₋₃)alkyl, each substituted or unsubstituted.

According to the above variations, there is provided a compound wherein R₁₇ is selected from the group consisting of styrenyl, naphthyl, 2-, 3- or 4-(C₁₋₅)alkylbenzyl, and 2-, 3- or 4-(C₁₋₅)alkoxybenzyl, each substituted or unsubstituted. In variations of the above, R₁₇ is selected from the group consisting of phenyl, methylenedioxyphenyl, biphenyl, 2-, 3- or 4-(C₁₋₅)alkylphenyl, 2-, 3-, or 4-halophenyl, 2- or 4-CN-phenyl, 3- or 4-NO₂-phenyl, 2-, 3- or 4-MeO-phenyl, 2-, 3-, or 4-CF₃-phenyl, 2-, 3-, or 4-CF₃O-phenyl, 2- or 4-(C₁₋₅)alkylSO₂phenyl, 2- or 4-(C₁₋₅)alkylSOphenyl, 2- or 4-[(C₁₋₄)alkylOCO]phenyl, 2,3-, 2,4-, 2,5- or 2,6-dihalophenyl, 3,4-dihalophenyl, 3,5-dihalophenyl, 2-, 3- or 4-carboxyphenyl, 2,3-, 2,4-, 2,5- or 2,6-, or 3,5-di(C₁₋₄alkylphenyl, 3,5-di(C₁₋₄)alkoxyphenyl, 3,5-di(CF₃)-phenyl, perfluoro(C₁₋₄)alkylphenyl, and F₅-phenyl, each further substituted or unsubstituted.

According to variations of the above, R₁₇ is selected from the group consisting of (C₁₋₅)alkylHNCH₂—, arylNHCH₂—, alkylaryl HNCH₂—, heteroarylHNCH₂—, and alkylheteroarylHNCH₂—, each substituted or unsubstituted. According to another variation of the above, R₁₇ is selected from the group consisting of (C₁₋₅)alkylNH—, ((C₁₋₅)alkyl)₂N, ((C₁₋₅)alkyl)₂NCH₂—, phenyl-NH—, phenyl-N(C₁₋₅)alkyl-, 2-, 3-, or 4-halophenyl-NH—, and 2-, 3-, or 4-(C₁₋₅)alkylphenyl-NH—, each substituted or unsubstituted.

According to yet another variation of the above, there is provided a compound wherein R₁₇ is selected from the group consisting of styrenyl-CH₂—, and naphthyl-CH₂—, each substituted or unsubstituted. In yet another variation of the above, R₁₇ is selected from the group consisting of benzyl, methylenedioxybenzyl, biphenyl-CH₂—, 2-, 3- or 4-(C₁₋₅)alkylbenzyl, 2-, 3-, or 4-halobenzyl, 2- or 4-CN-benzyl, 3- or 4-NO₂-benzyl, 2-, 3- or 4-MeO-benzyl, 2-, 3-, or 4-CF₃-benzyl, 2-, 3-, or 4-CF₃O-benzyl, 2- or 4-(C₁₋₅)alkylSO₂benzyl, 2- or 4-[(C₁₋₄)alkylOCO]benzyl, 2,3-, 2,4-, 2,5- or 2,6-dihalobenzyl, 3,4-dihalobenzyl, 3,5-dihalobenzyl, 2-, 3- or 4-carboxybenzyl, 2,3-, 2,4-, 2,5- or 2,6-, or 3,5-di(C₁₋₄)alkylbenzyl, 3,5-di(C₁₋₄)alkoxybenzyl, 3,5-di(CF₃)-benzyl, perfluoro(C₁₋₄)alkylbenzyl, and F₅-benzyl, each further substituted or unsubstituted.

According to yet another variation of the above, R₁₇ is selected from the group consisting of (C₁₋₅)alkylNH-CH₂—, ((C₁₋₅)alkyl)₂NCH₂—, phenyl-NH—CH₂—, phenyl-N(C₁₋₅)alkyl-, 2-, 3-, or 4-halophenyl-NH—CH₂—, and 2-, 3-, or 4-(C₁₋₅)alkylphenyl-NH—CH₂—, each substituted or unsubstituted.

In yet another variation of the above, R₁₇ is selected from the group consisting of 2-, 3- or 4-pyridyl, 2-(C₁₋₄)alkyl-pyrid-5-yl, imidazolyl, pyrazolyl, quinolinyl, quinoxalinyl, quinazolinyl, pyrrolyl, pyrazolyl, benzofuranyl, benzothiophenyl, benzimidazolyl, indolyl, furanyl, and tetrahydrofuranyl, each substituted or unsubstituted. In another variation, R₁₇ is selected from the group consisting of halo(C₁₋₆)alkyl, cycloalkyl, heterocycloalkyl, aryl, heteroaryl, carbonyl(C₁₋₅)alkyl, thiocarbonyl (C₁₋₅)alkyl, sulfonyl(C₁₋₃)alkyl, sulfinyl(C₁₋₃)alkyl, and imino(C₁₋₃)alkyl, each substituted or unsubstituted.

According to the above variations, there is provided a compound wherein R₁₇ is selected from the group consisting of (C₁₋₁₀)alkyl, halo(C₁₋₆)alkyl, aryl(C₁₋₁₀)alkyl, cycloalkyl, aryl, heteroaryl, (C₁₋₁₀)alkylaryl, heteroaryl, heteroaryl(C₁₋₆)alkyl, hetero(C₃₋₁₂)cycloalkyl, and (C₁₋₁₀)alkylhetero(C₃₋₈)aryl, each substituted or unsubstituted, and wherein R₁₁ is hydrogen or is selected from the group consisting of (C₁₋₁₀)alkyl, halo(C₁₋₆)alkyl, aryl(C₁₋₁₀)alkyl, cycloalkyl, aryl, heteroaryl, (C₁₋₁₀)alkylaryl, heteroaryl, heterocycloalkyl, heteroaryl(C₁₋₆)alkyl, hetero(C₃₋₁₂)cycloalkyl, and (C₁₋₁₀)alkylhetero(C₃₋₈)aryl, each substituted or unsubstituted.

In the above variations, there is provided a compound wherein R₁₇ is selected from the group consisting of (C₁₋₁₀)alkyl, halo(C₁₋₆)alkyl, aryl(C₁₋₁₀)alkyl, cycloalkyl, aryl, heteroaryl, (C₁₋₁₀)alkylaryl, heteroaryl, hetero(C₃₋₁₂)cycloalkyl, heteroaryl(C₁₋₆)alkyl, hetero(C₃₋₁₂)cycloalkyl, and (C₁₋₁₀)alkylhetero(C₃₋₈)aryl, each substituted or unsubstituted. In yet another variation of the above, R₁₇ is selected from the group consisting of (C₁₋₁₀)alkyl, aryl(C₁₋₁₀)alkyl, aryl, heteroaryl, heteroaryl(C₁₋₆)alkyl, and hetero(C₃₋₁₂)cycloalkyl, each substituted or unsubstituted. In yet another variation of the above, R₁₇ is selected from the group consisting of (C₁₋₃)alkyl, phenyl(C₁₋₃)alkyl, phenyl, biphenyl, naphthyl, naphthyl(C₁₋₃)alkyl, heteroaryl, and heteroaryl(C₁₋₆)alkyl, each substituted or unsubstituted.

According to the above variations, there is provided a compound wherein R₁₇ is selected from the group consisting of 3- or 4-halophenyl, 2-(C₁₋₃)alkyl-5-halophenyl, 2-, 3- or 4-halophenyl, 2-, 3- or 4-halobenzyl, 2,3-, 2,4-, 2,5-, or 3,4-dihalophenyl, 4-cyanophenyl, 4-(pyrrolidiny-1-yl)phenyl, 4-(C₁₋₃)alkylSO₂phenyl, 4-(C₁₋₃)alkylSOphenyl, 4-(C₁₋₆)alkoxyphenyl, 2-, 3- or 4-(C₁₋₃)alkylthiophenyl, 4-CF₃-thiophenyl, 4-(C₁₋₃)alkylNH-(C₁₋₃)alkylphenyl, 4-morpholin-4-yl-(C₁₋₃)alkylphenyl, and 4-(piperidin-1-yl-(C₁₋₃)alkyl)phenyl, each unsubstituted or further substituted. In another variation of the above, R₁₇ is selected from the group consisting of 2-, 3- or 4-(C₁₋₃)alkylphenyl, 2-, 3- or 4-(C₁₋₃)alkylbenzyl, 8-halo-naphthalen-1-yl, napthalen-1-yl-CH═CH—, 2-(C₁₋₃)alkylphenyl-CH═CH—, 2,3-dihalophenyl-CH═CH—, 2,5-dihalophenyl, 2,6-dihalophenyl-CH═CH—, 2-halo-4,5-dimethoxyphenyl, 2-halo-3,4-dimethoxyphenyl, 3-halo-4-methoxyphenyl, 3-halo-4-(C₁₋₃)alkylphenyl, 2-halo-5-trifluoromethylphenyl, 4-trifluoromethoxyphenyl, 2-, 3- or 4 (C₁₋₃)alkoxyphenyl, phenylcyclopropyl-, 2-, 3- or 4-(C₁₋₃)alkoxybenzyl, 3- or 4-cyanophenyl, 3- or 4-cyanobenzyl, 2- or 3-pyridyl, 2- or 3-furanyl, 2- or 3-thiophenyl, 3-halo-thiophen-2-yl, thieno[2,3-b]thiophenyl, 4,5-dihalo-isothiazole-3-yl, 3,6-dihalo-benzo[b]thiophen-2-yl, 5-halo-3-(C₁₋₃)alkyl-benzo [b]thiophen-2-yl, 5-halo-thiophen-2-yl, 3-bromo-5-chloro-thiophen-2-yl, and 3-halo-benzo[b]thiophen-2-yl, each further substituted or unsubstituted.

### Substituent R₁₈:

In particular variations of the above, there is provided a compound wherein R₁₈ is selected from the group consisting of hydrogen, nitro, cyano, thio, hydroxy, alkoxy, carbonyl, amino, (C₁₋₁₀)alkylamino, sulfonamido, imino, sulfonyl, sulfinyl, (C₁₋₁₀)alkyl, (C₃₋₇)cycloalkyl, hetero(C₃₋₇)cycloalkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted. In another variation, R₁₈ is selected from the group consisting of hydrogen, halo, and (C₁₋₆)alkyl, each substituted or unsubstituted.

### Substituent R₂₃, R₂₄, R₂₅ and R₂₆:

In one variation of the above, there is provided a compound wherein R₂₃, R₂₄, R₂₅, and R₂₆ are each independently selected from the group consisting of hydrogen, (C₁₋₁₀)alkylamino, (C₃₋₇)cycloalkyl, hetero(C₃₋₇)cycloalkyl, halo(C₁₋₁₀)alkyl, aryl, and heteroaryl, each substituted or unsubstituted.

### Substituent L:

In one aspect of the invention, L is a linker preferably comprising a chain having at least 1 atom. In one variation, the linker comprises at least 1 carbon atom. The linker chain atoms, i.e., the atoms defining the backbone of the linker chain, are selected from the group consisting of C, O, N, and S. In one aspect, the linker chain atoms are selected from the group consisting of C, O, and N, with no oxygen atom being directly bonded to another oxygen atom or to a nitrogen atom of the linker chain or any other nitrogen atom. In another aspect, the linker chain atoms are selected from the group consisting of C and N. In one variation, the linker comprises a chain having about 1 to about 5 chain atoms. In another variation, the linker comprises a chain of 2 to 4 carbon atoms.

According to particular variations of the above compounds, L is a substituted or unsubstituted (C₁₋₆)alkyl. In one variation, L is selected from the group consisting of methyl, ethyl, and propyl, each unsubstituted or substituted. In another variation, L is a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl, each further substituted or unsubstituted.

In one variation of the above, Lₐ is an unsubstituted or substituted (C₁₋₆)alkyl. In another variation, Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each further substituted or unsubstituted.

According to particular variations of the above, L is selected from the group consisting of —CH₂—, —(CH₂)₂—, —(CH₂)₃—, —(CH₂)₄—, —(CH₂)₅—, and —(CH₂)₆—, each substituted or unsubstituted. In yet another variation of the above, L is selected from the group consisting of arylene, (C₁₋₆)alkylarylene, aryl(C₁₋₆)alkylene, heteroarylene, and (C₁₋₆)alkylhetero(C₃₋₈)arylene, and hetero(C₃₋₈)aryl(C₁₋₆)alkylene, each substituted or unsubstituted.

### Particular Mitotic Kinesin Inhibitors:

Particular examples of mitotic kinesin inhibitors according to the present invention include, but are not limited to:

(R)-{3-[1-(1-Benzyl-1H-benzoimidazol-2-yl)-2-methyl-propylamino]-propyl}-carbamic acid tert-butyl ester;

(R)—N-(3-amino-propyl)-N-[1-(1-benzyl-1H-benzoimidazol-2-yl)-2-methyl-propyl]-4-methyl-benzamide;

N-(3-Amino-propyl)-N-(1-benzyl-1H-benzoimidazol-2-ylmethyl)-4-methyl-benzamide;

(R)—N-(3-Amino-propyl)-N-[1-(1-benzyl-1H-benzoimidazol-2-yl)-ethyl]-4-methyl-benzamide;

(R)—N-(3-Amino-propyl)-N-[1-(1-benzyl-1H-benzoimidazol-2-yl)-propyl]-4-methyl-benzamide;

(R)—N-(3-Amino-propyl)-N-]1-(1-benzyl-1H-benzoimidazol-2-yl)-2-methyl-propyl]-4-methyl-benzamide;

(R)—N-(3-Amino-propyl)-N-[1-(1-benzyl-7-chloro-1H-benzoimidazol-2-yl)-2-methyl-propyl]-4-methyl-benzamide;

(R)—N-(3-Amino-propyl)-N-[1-(1-benzyl-6-chloro-1H-benzoimidazol-2-yl)-2-methyl-propyl]-4-methyl-benzamide;

(R)—N-(3-Amino-propyl)-N-[1-(1-benzyl-5-chloro-1H-benzoimidazol-2-yl)-2-methyl-propyl]-4-methyl-benzamide;

(R)—N-(3-Amino-propyl)-N-[1-(1-benzyl-5,6-dichloro-1H-benzoimidazol-2-yl)-2-methyl-propyl]-4-methyl-benzamide;

(R)—N-(3-Amino-propyl)-N-[1-(1-benzyl-7-methyl-1H-benzoimidazol-2-yl)-2-methyl-propyl]-4-methyl-benzamide;

(R)—N-(3-Amino-propyl)-N-[1-(1-benzyl-6-methyl-1H-benzoimidazol-2-yl)-2-methyl-propyl]-4-methyl-benzamide;

(R)—N-(3-Amino-propyl)-N-[1-(1-benzyl-5,6-dimethyl-1H-benzoimidazol-2-yl)-2-methyl-propyl]-4-methyl-benzamide;

(R)—N-(3-Amino-propyl)-N-[1-(1-benzyl-1H-benzoimidazol-2-yl)-2-methyl-propyl]-3-methyl-benzamide;

(R)—N-(3-Amino-propyl)-N-[1-(1-benzyl-1H-benzoimidazol-2-yl)-2-methyl-propyl]-2-methyl-benzamide;

(R)—N-(3-Amino-propyl)-N-[1-(1-benzyl-1H-benzoimidazol-2-yl)-2-methyl-propyl]-4-chloro-benzamide;

(R)—N-(3-Amino-propyl)-N-[1-(1-benzyl-1H-benzoimidazol-2-yl)-2-methyl-propyl]-3-chloro-benzamide;

(R)—N-(3-Amino-propyl)-N-[1-(1-benzyl-1H-benzoimidazol-2-yl)-2-methyl-propyl]-2-chloro-benzamide;

(R)—N-(3-Amino-propyl)-N-[1-(1-benzyl-1H-benzoimidazol-2-yl)-2-methyl-propyl]-4-methoxy-benzamide;

(R)—N-(3-Amino-propyl)-N-[1-(1-benzyl-1H-benzoimidazol-2-yl)-2-methyl-propyl]-3-methoxy-benzamide;

(R)—N-(3-Amino-propyl)-N-[1-(1-benzyl-1H-benzoimidazol-2-yl)-2-methyl-propyl]-2-methoxy-benzamide;

(R)—N-(3-Amino-propyl)-N-[4-(1-benzyl-1H-benzoimidazol-2-yl)-2-methyl-propyl]-4-cyano-benzamide;

(R)—N-(3-Amino-propyl)-N-[1-(1-benzyl-1H-benzoimidazol-2-yl)-2-methyl-propyl]-3-cyano-benzamide;

(R)—N-(3-Amino-propyl)-N-[1-(1-benzyl-1H-benzoimidazol-2-yl)-2-methyl-propyl]-nicotinamide;

(R)-Pyridine-2-carboxylic acid (3-amino-propyl)-[1-(1-benzyl-1H-benzoimidazol-2-yl)-2-methyl-propyl]-amide;

(R)-Furan-2-carboxylic acid (3-amino-propyl)-[1-(1-benzyl-1H-benzoimidazol-2-yl)-2-methyl-propyl]-amide;

(R)-Thiophene-2-carboxylic acid (3-amino-propyl)-[1-(1-benzyl-1H-benzo-imidazol-2-yl)-2-methyl-propyl]-amide;

(R)—N-(3-Amino-propyl)-N-[1-(1-benzyl-1H-benzoimidazol-2-yl)-2-methyl-propyl]-2-phenyl-acetamide;

(R)—N-(3-Amino-propyl)-N-[1-(1-benzyl-1H-benzoimidazol-2-yl)-2-methyl-propyl]-3-phenyl-propionamide;

(R)—N-(3-Amino-propyl)-N-[1-(1-benzyl-1H-benzoimidazol-2-yl)-2-methyl-propyl]-4-ethyl-benzamide;

(R)—N-(3-Amino-propyl)-4-methyl-N-[2-methyl-1-(1-methyl-1H-benzoimidazol-2-yl)-propyl]-benzamide;

(S)—N-(3-Amino-propyl)-N-[1-(1-benzyl-1H-benzoimidazol-2-yl)-2-methyl-propyl]-4-methyl-benzamide;

(R)—N-(3-aminopropyl)-N-(1-(1-benzyl-4-chloro-1H-benzokllimidazol-2-yl)-2-methylpropyl)-4-methylbenzamide;

(R)—N-(3-aminopropyl)-N-(1-(1-benzyl-1H-benzo[d]imidazol-2-yl)-2-methylpropyl)-5-ethylthiophene-3-carboxamide;

(R)—N-(3-aminopropyl)-N-(1-(1-benzyl-1H-benzo[d]imidazol-2-yl)-2-methylpropyl)-4-bromo-5-ethylthiophene-2-carboxamide;

(R)—N-(3-aminopropyl)-N-(1-(1-benzyl-1H-benzo[d]imidazol-2-yl)-2-methylpropyl)-4-bromo-5-propylthiophene-2-carboxamide;

(R)—N-(3-aminopropyl)-N-(1-(1-benzyl-1H-benzo[d]imidazol-2-yl)-2-methylpropyl)-5-methylthiophene-2-carboxamide;

(R)—N-(3-aminopropyl)-N-(1-(1-benzyl-1H-benzo]d]imidazol-2-yl)-2-methylpropyl)-5-isopropylthiophene-3-carboxamide;

(R)—N-(3-aminopropyl)-N-(1-(1-benzyl-1H-benzo[d]imidazol-2-yl)-2-methylpropyl)-5-(methylthio)thiophene-2-carboxamide;

(R)—N-(3-aminopropyl)-N-(1-(1-benzyl-1H-benzo[d]imidazol-2-yl)-2-methylpropyl)-4,5-dimethylthiophene-2-carboxamide;

(R)—N-(3-aminopropyl)-N-(1-(1-benzyl-1H-benzo[d]imidazol-2-yl)-2-methylpropyl)-4-bromothiophene-2-carboxamide;

(R)—N-(3-aminopropyl)-N-(1-(1-benzyl-1H-benzo[d]imidazol-2-yl)-2-methylpropyl)-4,5-dibromothiophene-2-carboxamide;

(R)—N-(3-aminopropyl)-N-(1-(1-benzyl-1H-benzo[d]imidazol-2-yl)-2-methylpropyl)thieno[2,3-b]thiophene-2-carboxamide;

(R)—N-(3-aminopropyl)-N-(1-(1-benzyl-4-methyl-1H-benzo[d]imidazol-2-yl)-2-methylpropyl)-4-methylbenzamide;

(R)—N-(3-aminopropyl)-N-(1-(1-benzyl-5-methyl-1H-benzo[d]imidazol-2-yl)-2-methylpropyl)-4-methylbenzamide;

(R)—N-(3-aminopropyl)-N-(1-(1-benzyl-4,5-dimethyl-1H-benzo[d]imidazol-2-yl)-2-methylpropyl)-4-methylbenzamide;

(R)—N-(3-aminopropyl)-N-(1-(1-benzyl-4,5-dichloro-1H-benzo[d]imidazol-2-yl)-2-methylpropyl)-4-methylbenzamide;

(R)—N-(3-aminopropyl)-N-(1-(1-benzyl-4-chloro-5-methyl-1H-benzo[d]imidazol-2-yl)-2-methylpropyl)-4-methylbenzamide ;

(R)—N-(3-aminopropyl)-N-(1-(1-benzyl-5-chloro-4-methyl-1H-benzo[d]imidazol-2-yl)-2-methylpropyl)-4-methylbenzamide;

(R)—N-(3-aminopropyl)-N-(1-(1-benzyl-4-ethyl-1H-benzo[d]imidazol-2-yl)-2-methylpropyl)-4-methylbenzamide;

(R)—N-(3-aminopropyl)-N-(1-(1-benzyl-5-ethyl-1H-benzo[d]imidazol-2-yl)-2-methylpropyl)-4-methylbenzamide;

(R)—N-(3-aminopropyl)-N-(1-(1-benzyl-4-ethyl-5-methyl-1H-benzo[d]imidazol-2-yl)-2-methylpropyl)-4-methylbenzamide ;

(R)—N-(3-aminopropyl)-N-(1-(1-benzyl-5-chloro-4-ethyl-1H-benzo[d]imidazol-2-yl)-2-methylpropyl)-4-methylbenzamide;

(R)—N-(3-aminopropyl)-N-(1-(1-benzyl-5-ethyl-4-methyl-1H-benzo[d]imidazol-2-yl)-2-methylpropyl)-4-methylbenzamide;

(R)—N-(3-aminopropyl)-N-(1-(1-benzyl-4-chloro-5-ethyl-1H-benzo[d]imidazol-2-yl)-2-methylpropyl)-4-methylbenzamide ;

(R)—N-(3-aminopropyl)-N-(1-(1-benzyl-4-cyano-1H-benzo[d]imidazol-2-yl)-2-methylpropyl)-4-methylbenzamide;

(R)—N-(2-aminoethyl)-N-(1-(1-benzyl-1H-benzo[d]imidazol-2-yl)-2-methylpropyl)-4-methylbenzamide;

(R)—N-(1-(1-benzyl-1H-benzo[d]imidazol-2-yl)-2-methylpropyl)-N-ethyl-4-methylbenzamide;

(R)—N-(1-(1-benzyl-1H-benzo[d]imidazol-2-yl)-2-methylpropyl)-4-methyl-N-propylbenzamide;

(R)—N-(1-(1-benzyl-1H-benzo[d]imidazol-2-yl)-2-methylpropyl)-N-(cyclopropylmethyl)-4-methylbenzamide;

(R)—N-(3-aminopropyl)-N-(1-(1-benzyl-1H-benzo[d]imidazol-2-yl)-2-methylpropyl)-6-methylnicotinamide;

(R)—N-(3-aminopropyl)-N-(1-(1-benzyl-1H-benzo[d]imidazol-2-yl)-2-methylpropyl)-6-chloronicotinamide;

(R)—N-(3-aminopropyl)-4-methyl-N-(2-methyl-1-(1-(thiophen-2-ylmethyl)-1H-benzo[d]imidazol-2-yl )propyl)benzamide;

(R)—N-(3-aminopropyl)-N-(1-(3-benzyl-3H-imidazo[4,5-b]pyridin-2-yl)-2-methylpropyl)-4-methylbenzamide;

(R)—N-(3-aminopropyl)-4-methyl-N-(2-methyl-1-(1-(pyridin-3-ylmethyl)-1H-benzo[d]imidazol-2-yl)propyl)benzamide;

(R)—N-(3-aminopropyl)-N-(1-(9-benzyl-9H-purin-8-yl)-2-methylpropyl)-4-methylbenzamide;

(R)—N-(3-aminopropyl)-N-(1-(3-benzyl-7-chloro-3H-imidazo[4,5-c]pyridin-2-yl)-2-methylpropyl)-4-methylbenzamide; and

(R)—N-(1-(3-benzyl-7-chloro-3H-imidazo[4,5-c]pyridin-2-yl)-2-methylpropyl)-N-(cyclopropylmethyl)-4-methylbenzamide.

According to each of the above, there is provided the above compound in the form of a pharmaceutically acceptable salt. In one variation, the compound is present in a mixture of stereoisomers. In another variation, the compound comprises a single stereoisomer. In a particular variation, there is provided a pharmaceutical composition comprising, as an active ingredient, a compound according to any of the above variations. In another variation of the pharmaceutical composition, the composition is a solid formulation adapted for oral administration. In one variation of the above composition, the composition is a tablet. In another variation, the composition is a liquid formulation adapted for oral administration. In yet another variation, the composition is a liquid formulation adapted for parenteral administration.

According to the above variations, there is provided a pharmaceutical composition comprising a compound wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, and intrathecally.

In one aspect, there is provided a kit comprising a compound according to any one of the above compounds and instructions which comprise one or more forms of information selected from the group consisting of indicating a disease state for which the compound is to be administered, storage information for the compound, dosing information and instructions regarding how to administer the compound. In one aspect, the kit comprises the compound in a multiple dose form.

In another aspect, there is provided an article of manufacture comprising a compound according to those described above and packaging materials. In one variation of the above, the packaging material comprises a container for housing the compound. In another variation, the container comprises a label indicating one or more members of the group consisting of a disease state for which the compound is to be administered, storage information, dosing information and/or instructions regarding how to administer the composition. In one particular aspect of the above, the article of manufacture comprises the compound in a multiple dose form.

According to one aspect, there is provided a method of treating cellular proliferative diseases comprising administering a compound as described above.

According to another aspect, there is provided a method of treating a disorder associated with KSP kinesin activity comprising administering any one of the above compounds.

According to another aspect, there is provided a method of inhibiting KSP kinesin comprising contacting KSP kinesin with any of the above compounds. In one aspect of the above, the disease or disorder is selected from the group consisting of cancer, hyperplasia, restenosis, cardiac hypertrophy, immune disorders and inflammation.

According to one aspect, there is provided a method of inhibiting KSP kinesin comprising contacting KSP kinesin with a compound comprising the formula:

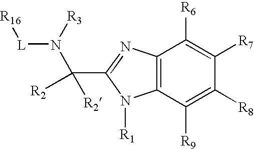

wherein:

R₁ is hydrogen, or is selected from the group consisting of (C₁₋₆)alkyl, (C₁₋₆)alkyloxy(C₁₋₆)alkyl, aryl, (C₁₋₆)alkyl aryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;

R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;

R₃ is selected from the group consisting of hydrogen, halo(C₁₋₆)alkyl, (C₁₋₁₅)alkyl, cycloalkyl, hetero(C₃₋₁₂)cycloalkyl, aryl, heteroaryl, carbonyl(C₁₋₅)alkyl, thiocarbonyl (C₁₋₅)alkyl, sulfonyl(C₁₋₃)alkyl, sulfinyl(C₁₋₃)alkyl, imino(C₁₋₃)alkyl, carbonyl group, imino group, sulfonyl group, sulfinyl group and sulfonamido, each substituted or unsubstituted, or R₃ and R₂ or R₂′ are taken together to form a 4- to 7-membered substituted or unsubstituted ring;

R₆, R₇, R₈, and R₉ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;

R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl (C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring; and

L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

It is noted that the compounds of the present invention may be in the form of a pharmaceutically acceptable salt, biohydrolyzable ester, biohydrolyzable amide, biohydrolyzable carbamate, solvate, hydrate or prodrug thereof. For example, the compound optionally comprises a substituent that is convertible in vivo to a different substituent such as a hydrogen.

It is further noted that the compounds of the present invention may optionally be solely or predominantly in the enol tautomer in its active state. It is further noted that the compound may be present in a mixture of stereoisomers, or the compound comprises a single stereoisomer.

The invention also provides pharmaceutical compositions comprising, as an active ingredient, a compound according to any one of the above embodiments and variations. In addition, the composition may be a solid or liquid formulation adapted for oral administration. In a further variation, the pharmaceutical composition may be a tablet. In yet another variation, the pharmaceutical composition may be a liquid formulation adapted for parenteral administration.

In one embodiment, there is provided the pharmaceutical composition comprising a compound according to any one of the above embodiments and variations wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

The invention also provides a kit comprising a compound or composition according to any one of the above embodiments and variations, and instructions which comprise one or more forms of information selected from the group consisting of indicating a disease state for which the compound is to be administered, storage information for the compound, dosing information and instructions regarding how to administer the compound. In one variation, the kit comprises the compound or composition in a multiple dose form.

In another embodiment, the present invention provides an article of manufacture comprising a compound or composition according to any one of the above embodiments and variations, and packaging materials. In one variation, the packaging material comprises a container for housing the compound or composition. The container optionally comprises a label indicating a disease state for which the compound or composition is to be administered, storage information, dosing information and/or instructions regarding how to administer the compound or composition. In regard to the above embodiments and variations, the article of manufacture optionally comprises the compound or composition in a multiple dose form.

In another embodiment, the present invention provides a therapeutic method comprising administering a compound or composition according to any one of the above embodiments and variations to a subject.

In yet another embodiment, the present invention provides a method of inhibiting mitotic kinesins comprising contacting a mitotic kinesin with a compound or composition according to any one of the above embodiments and variations.

In still another embodiment, there is provided a method of inhibiting a mitotic kinesin comprising causing a compound or composition according to any one of the above embodiments and variations to be present in a subject in order to inhibit mitotic kinesin in vivo.

The present invention also provides a method of inhibiting a mitotic kinesin comprising administering a first compound to a subject that is converted in vivo to a second compound wherein the second compound inhibits mitotic kinesin in vivo, the second compound being a compound according to any one of the above embodiments and variations.

In yet another embodiment, there is provided a method of preventing or treating a disease state for which a mitotic kinesin possesses activity that contributes to the pathology and/or symptomology of the disease state comprising causing a compound or composition according to any one of the above embodiments and variations to be present in a subject in a therapeutically effective amount for the disease state.

The present invention also provides a method of preventing or treating a disease state for which a mitotic kinesin possesses activity that contributes to the pathology and/or symptomology of the disease state comprising administering a first compound to a subject that is converted in vivo to a second compound according to any one of the above embodiments and variations wherein the second compound is present in a subject in a therapeutically effective amount for the disease state.

In addition, there is provided a method of preventing or treating a disease state for which a mitotic kinesin possesses activity that contributes to the pathology and/or symptomology of the disease state comprising administering a compound or composition according to any one of the above embodiments and variations, wherein the compound or composition is present in the subject in a therapeutically effective amount for the disease state.

In each of the above embodiments and variations, the mitotic kinesin is optionally kinesin-like spindle protein (IPSP).

### Salts, Hydrates, and Prodrugs of Mitotic Kinesins Inhibitors:

It should be recognized that the compounds of the present invention may be present and optionally administered in the form of salts, hydrates and prodrugs that are converted in vivo into the compounds of the present invention. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids and their corresponding salts such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g. potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine(benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

Compounds of the present invention that comprise basic nitrogen-containing groups may be quaternized with such agents as (C₁₋₄)alkylhalides, e.g., methyl, ethyl, iso-propyl and tert-butyl chlorides, bromides and iodides; di(C₁₋₄)alkyl sulfates, e.g., dimethyl, diethyl and diamyl sulfates; (C₁₀₋₁₈)alkyl halides, e.g., decyl, dodecyl, lauryl, myristyl and stearyl chlorides, bromides and iodides; and aryl(C₁₋₄)alkylhalides, e.g., benzyl chloride and phenethyl bromide. Such salts permit the preparation of both water-soluble and oil-soluble compounds of the present invention.

N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Prodrug derivatives of compounds according to the present invention can be prepared by modifying substituents of compounds of the present invention that are then converted in vivo to a different substituent. It is noted that in many instances, the prodrugs themselves also fall within the scope of the range of compounds according to the present invention. For example, prodrugs can be prepared by reacting a compound with a carbamylating agent (e.g., 1,1-acyloxyalkylcarbonochloridate, para-nitrophenyl carbonate, or the like) or an acylating agent. Further examples of methods of making prodrugs are described in Saulnier et al. (1994), Bioorganic and Medicinal Chemistry Letters, Vol. 4, p. 1985.

Protected derivatives of compounds of the present invention can also be made. Examples of techniques applicable to the creation of protecting groups and their removal can be found in T. W. Greene, Protecting Groups in Organic Synthesis, 3^{rd} edition, John Wiley & Sons, Inc. 1999.

Compounds of the present invention may also be conveniently prepared, or formed during the process of the invention, as solvates (e.g. hydrates). Hydrates of compounds of the present invention may be conveniently prepared by recrystallization from an aqueous/organic solvent mixture, using organic solvents such as dioxin, tetrahydrofuran or methanol.

A “pharmaceutically acceptable salt”, as used herein, is intended to encompass any compound according to the present invention that is utilized in the form of a salt thereof, especially where the salt confers on the compound improved pharmacokinetic properties as compared to the free form of compound or a different salt form of the compound. The pharmaceutically acceptable salt form may also initially confer desirable pharmacokinetic properties on the compound that it did not previously possess, and may even positively affect the pharmacodynamics of the compound with respect to its therapeutic activity in the body. An example of a pharmacokinetic property that may be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

### Preparation of Mitotic Kinesin Inhibitors:

Various methods may be developed for synthesizing compounds according to the present invention. Representative methods for synthesizing these compounds are provided in the Examples. It is noted, however, that the compounds of the present invention may also be synthesized by other synthetic routes that others may devise.

It will be readily recognized that certain compounds according to the present invention have atoms with linkages to other atoms that confer a particular stereochemistry to the compound (e.g., chiral centers). It is recognized that synthesis of compounds according to the present invention may result in the creation of mixtures of different stereoisomers (enantiomers, diastereomers). Unless a particular stereochemistry is specified, recitation of a compound is intended to encompass all of the different possible stereoisomers.

Various methods for separating mixtures of different stereoisomers are known in the art. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) that they can be readily separated by taking advantage of these dissimilarities. For example, diastereomers can typically be separated by chromatography or by separation/resolution techniques based upon differences in solubility. A more detailed description of techniques that can be used to resolve stereoisomers of compounds from their racemic mixture can be found in Jean Jacques Andre Collet, Samuel H. Wilen, Enantiomers, Racemates and Resolutions, John Wiley & Sons, Inc. (1981).

Compounds according to the present invention can also be prepared as a pharmaceutically acceptable acid addition salt by reacting the free base form of the compound with a pharmaceutically acceptable inorganic or organic acid. Alternatively, a pharmaceutically acceptable base addition salt of a compound can be prepared by reacting the free acid form of the compound with a pharmaceutically acceptable inorganic or organic base. Inorganic and organic acids and bases suitable for the preparation of the pharmaceutically acceptable salts of compounds are set forth in the definitions section of this Application. Alternatively, the salt forms of the compounds can be prepared using salts of the starting materials or intermediates.

The free acid or free base forms of the compounds can be prepared from the corresponding base addition salt or acid addition salt form. For example, a compound in an acid addition salt form can be converted to the corresponding free base by treating with a suitable base (e.g., ammonium hydroxide solution, sodium hydroxide, and the like). A compound in a base addition salt form can be converted to the corresponding free acid by treating with a suitable acid (e.g., hydrochloric acid, etc).

The N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Compounds in an unoxidized form can be prepared from N-oxides of compounds by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds can be prepared by methods known to those of ordinary skill in the art (e.g., for further details see Saulnier et al. (1994), Bioorganic and Medicinal Chemistry Letters, Vol. 4, p. 1985). For example, appropriate prodrugs can be prepared by reacting a non-derivatized compound with a suitable carbamylating agent (e.g., 1,1-acyloxyalkylcarbonochloridate, para-nitrophenyl carbonate, or the like).

Protected derivatives of the compounds can be made by methods known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds according to the present invention may be conveniently prepared, or formed during the process of the invention, as solvates (e.g. hydrates). Hydrates of compounds of the present invention may be conveniently prepared by recrystallization from an aqueous/organic solvent mixture, using organic solvents such as dioxin, tetrahydrofuran or methanol.

Compounds according to the present invention can also be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diastereomeric derivatives of compounds, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

Compositions Comprising Mitotic Kinesin Inhibitors:

A wide variety of compositions and administration methods may be used in conjunction with the mitotic kinesin inhibitors of the present invention. Such compositions may include, in addition to the mitotic kinesin inhibitors of the present invention, conventional pharmaceutical excipients, and other conventional, pharmaceutically inactive agents. Additionally, the compositions may include active agents in addition to the mitotic kinesin inhibitors of the present invention. These additional active agents may include additional compounds according to the invention, and/or one or more other pharmaceutically active agents.

The compositions may be in gaseous, liquid, semi-liquid or solid form, formulated in a manner suitable for the route of administration to be used. For oral administration, capsules and tablets are typically used. For parenteral administration, reconstitution of a lyophilized powder, prepared as described herein, is typically used.

Compositions comprising mitotic kinesins inhibitors of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or coadministered in slow release dosage forms.

The mitotic kinesins inhibitors and compositions comprising them may be administered or coadministered in any conventional dosage form. Co-administration in the context of this invention is intended to mean the administration of more than one therapeutic agent, one of which includes a mitotic kinesins inhibitor, in the course of a coordinated treatment to achieve an improved clinical outcome. Such co-administration may also be coextensive, that is, occurring during overlapping periods of time.

Solutions or suspensions used for parenteral, intradermal, subcutaneous, or topical application may optionally include one or more of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; agents for the adjustment of tonicity such as sodium chloride or dextrose, and agents for adjusting the acidity or alkalinity of the composition, such as alkaline or acidifying agents or buffers like carbonates, bicarbonates, phosphates, hydrochloric acid, and organic acids like acetic and citric acid. Parenteral preparations may optionally be enclosed in ampules, disposable syringes or single or multiple dose vials made of glass, plastic or other suitable material.

When mitotic kinesins inhibitors according to the present invention exhibit insufficient solubility, methods for solubilizing the compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate. Derivatives of the compounds, such as prodrugs of the compounds may also be used in formulating effective pharmaceutical compositions.

Upon mixing or adding mitotic kinesins inhibitors according to the present invention to a composition, a solution, suspension, emulsion or the like may be formed. The form of the resulting composition will depend upon a number of factors, including the intended mode of administration, and the solubility of the compound in the selected carrier or vehicle. The effective concentration needed to ameliorate the disease being treated may be empirically determined.

Compositions according to the present invention are optionally provided for administration to humans and animals in unit dosage forms, such as tablets, capsules, pills, powders, dry powders for inhalers, granules, sterile parenteral solutions or suspensions, and oral solutions or suspensions, and oil-water emulsions containing suitable quantities of the compounds, particularly the pharmaceutically acceptable salts, preferably the sodium salts, thereof. The pharmaceutically therapeutically active compounds and derivatives thereof are typically formulated and administered in unit-dosage forms or multiple-dosage forms. Unit-dose forms, as used herein, refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses that are not segregated in packaging.

In addition to one or more mitotic kinesins inhibitors according to the present invention, the composition may comprise: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to form a solution or suspension. If desired, the pharmaceutical composition to be administered may also contain minor amounts of auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known in the art, or will be apparent, to those skilled in this art; for example, see Remington's Pharmaceutical Sciences, Mack Publishing Company, Easton, Pa., 15th Edition, 1975. The composition or formulation to be administered will, in any event, contain a sufficient quantity of a mitotic kinesin inhibitor of the present invention to reduce mitotic kinesin activity in vivo, thereby treating the disease state of the subject.

Dosage forms or compositions may optionally comprise one or more mitotic kinesin inhibitors according to the present invention in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of one or more mitotic kinesin inhibitors, optionally 0.1-95%, and optionally 1-95%.

Salts, preferably sodium salts, of the mitotic kinesin inhibitors may be prepared with carriers that protect the compound against rapid elimination from the body, such as time release formulations or coatings. The formulations may further include other active compounds to obtain desired combinations of properties.

### Formulations for Oral Administration:

Oral pharmaceutical dosage forms may be as a solid, gel or liquid. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art.

In certain embodiments, mitotic kinesins inhibitors according to the present invention are provided as solid dosage forms, preferably capsules or tablets. The tablets, pills, capsules, troches and the like may optionally contain one or more of the following ingredients, or compounds of a similar nature: a binder; a diluent; a disintegrating agent; a lubricant; a glidant; a sweetening agent; and a flavoring agent.

Examples of binders that may be used include, but are not limited to, microcrystalline cellulose, gum tragacanth, glucose solution, acacia mucilage, gelatin solution, sucrose and starch paste.

Examples of lubricants that may be used include, but are not limited to, talc, starch, magnesium or calcium stearate, lycopodium and stearic acid.

Examples of diluents that may be used include, but are not limited to, lactose, sucrose, starch, kaolin, salt, mannitol and dicalcium phosphate.

Examples of glidants that may be used include, but are not limited to, colloidal silicon dioxide.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of coloring agents that may be used include, but are not limited to, any of the approved certified water soluble FD and C dyes, mixtures thereof; and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

If oral administration is desired, the salt of the compound may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Compounds according to the present invention may also be administered as a component of an elixir, suspension, syrup, wafer, sprinkle, chewing gum or the like. A syrup may optionally comprise, in addition to the active compounds, sucrose as a sweetening agent and certain preservatives, dyes and colorings and flavors.

The mitotic kinesins inhibitors of the present invention may also be mixed with other active materials that do not impair the desired action, or with materials that supplement the desired action.

Examples of pharmaceutically acceptable carriers that may be included in tablets comprising mitotic kinesin inhibitors of the present invention include, but are not limited to binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets may be compressed tablets to which different layers of pharmaceutically acceptable substances are applied. Film-coated tablets may be compressed tablets that have been coated with polymers or other suitable coating. Multiple compressed tablets may be compressed tablets made by more than one compression cycle utilizing the pharmaceutically acceptable substances previously mentioned. Coloring agents may also be used in tablets. Flavoring and sweetening agents may be used in tablets, and are especially useful in the formation of chewable tablets and lozenges.

Examples of liquid oral dosage forms that may be used include, but are not limited to, aqueous solutions, emulsions, suspensions, solutions and/or suspensions reconstituted from non-effervescent granules and effervescent preparations reconstituted from effervescent granules.

Examples of aqueous solutions that may be used include, but are not limited to, elixirs and syrups. As used herein, elixirs refer to clear, sweetened, hydroalcoholic preparations. Examples of pharmaceutically acceptable carriers that may be used in elixirs include, but are not limited to solvents. Particular examples of solvents that may be used include glycerin, sorbitol, ethyl alcohol and syrup. As used herein, syrups refer to concentrated aqueous solutions of a sugar, for example, sucrose. Syrups may optionally further comprise a preservative.

Emulsions refer to two-phase systems in which one liquid is dispersed in the form of small globules throughout another liquid. Emulsions may optionally be oil-in-water or water-in-oil emulsions. Examples of pharmaceutically acceptable carriers that may be used in emulsions include, but are not limited to non-aqueous liquids, emulsifying agents and preservatives.

Examples of pharmaceutically acceptable substances that may be used in non-effervescent granules, to be reconstituted into a liquid oral dosage form, include diluents, sweeteners and wetting agents.

Examples of pharmaceutically acceptable substances that may be used in effervescent granules, to be reconstituted into a liquid oral dosage form, include organic acids and a source of carbon dioxide.

Coloring and flavoring agents may optionally be used in all of the above dosage forms.

Particular examples of preservatives that may be used include glycerin, methyl and propylparaben, benzoic add, sodium benzoate and alcohol.

Particular examples of non-aqueous liquids that may be used in emulsions include mineral oil and cottonseed oil.

Particular examples of emulsifying agents that may be used include gelatin, acacia, tragacanth, bentonite, and surfactants such as polyoxyethylene sorbitan monooleate.

Particular examples of suspending agents that may be used include sodium carboxymethylcellulose, pectin, tragacanth, Veegum and acacia. Diluents include lactose and sucrose. Sweetening agents include sucrose, syrups, glycerin and artificial sweetening agents such as sodium cyclamate and saccharin.

Particular examples of wetting agents that may be used include propylene glycol monostearate, sorbitan monooleate, diethylene glycol monolaurate and polyoxyethylene lauryl ether.

Particular examples of organic acids that may be used include citric and tartaric acid.

Sources of carbon dioxide that may be used in effervescent compositions include sodium bicarbonate and sodium carbonate. Coloring agents include any of the approved certified water soluble FD and C dyes, and mixtures thereof.

Particular examples of flavoring agents that may be used include natural flavors extracted from plants such fruits, and synthetic blends of compounds that produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g. water, to be easily measured for administration.

Alternatively, liquid or semi-solid oral formulations may be prepared by dissolving or dispersing the active compound or salt in vegetable oils, glycols, triglycerides, propylene glycol esters (e.g. propylene carbonate) and other such carriers, and encapsulating these solutions or suspensions in hard or soft gelatin capsule shells. Other useful formulations include those set forth in U.S. Pat. Nos. Re 28,819 and 4,358,603.

### Injectables, Solutions, and Emulsions:

The present invention is also directed to compositions designed to administer the mitotic kinesin inhibitors of the present invention by parenteral administration, generally characterized by injection, either subcutaneously, intramuscularly or intravenously. Injectables may be prepared in any conventional form, for example as liquid solutions or suspensions, solid forms suitable for solution or suspension in liquid prior to injection, or as emulsions.

Examples of excipients that may be used in conjunction with injectables according to the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may be used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that may be used include sodium chloride and dextrose. Examples of buffers that may be used include phosphate and citrate. Examples of antioxidants that may be used include sodium bisulfate. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 80). A sequestering or chelating agent of metal ions include EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of a mitotic kinesins inhibitor in the parenteral formulation may be adjusted so that an injection administers a pharmaceutically effective amount sufficient to produce the desired pharmacological effect. The exact concentration of a mitotic kinesin inhibitor and/or dosage to be used will ultimately depend on the age, weight and condition of the patient or animal as is known in the art.

Unit-dose parenteral preparations may be packaged in an ampoule, a vial or a syringe with a needle. All preparations for parenteral administration should be sterile, as is know and practiced in the art.

Injectables may be designed for local and systemic administration. Typically a therapeutically effective dosage is formulated to contain a concentration of at least about 0.1% w/w up to about 90% w/w or more, preferably more than 1% w/w of the mitotic kinesins inhibitor to the treated tissue(s). The mitotic kinesins inhibitor may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The mitotic kinesin inhibitors may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

### Lyophilized Powders

The mitotic kinesin inhibitors of the present invention may also be prepared as lyophilized powders, which can be reconstituted for administration as solutions, emulsions and other mixtures. The lyophilized powders may also be formulated as solids or gels.

Sterile, lyophilized powder may be prepared by dissolving the compound in a sodium phosphate buffer solution containing dextrose or other suitable excipient. Subsequent sterile filtration of the solution followed by lyophilization under standard conditions known to those of skill in the art provides the desired formulation. Briefly, the lyophilized powder may optionally be prepared by dissolving dextrose, sorbitol, fructose, corn syrup, xylitol, glycerin, glucose, sucrose or other suitable agent, about 1-20%, preferably about 5 to 15%, in a suitable buffer, such as citrate, sodium or potassium phosphate or other such buffer known to those of skill in the art at, typically, about neutral pH. Then, a mitotic kinesin inhibitor is added to the resulting mixture, preferably above room temperature, more preferably at about 30-35° C., and stirred until it dissolves. The resulting mixture is diluted by adding more buffer to a desired concentration. The resulting mixture is sterile filtered or treated to remove particulates and to insure sterility, and apportioned into vials for lyophilization. Each vial may contain a single dosage or multiple dosages of the mitotic kinesin inhibitor.

### Topical Administration:

The mitotic kinesin inhibitors of the present invention may also be administered as topical mixtures. Topical mixtures may be used for local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The mitotic kinesin inhibitors may be formulated as aerosols for topical application, such as by inhalation (see, U.S. Pat. Nos. 4,044,126, 4,414,209, and 4,364,923, which describe aerosols for delivery of a steroid useful for treatment inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The mitotic kinesin inhibitors may also be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the mitotic kinesin inhibitor alone or in combination with other pharmaceutically acceptable excipients can also be administered.

Formulations for other Routes of Administrations:

Depending upon the disease state being treated, other routes of administration, such as topical application, transdermal patches, and rectal administration, may also be used. For example, pharmaceutical dosage forms for rectal administration are rectal suppositories, capsules and tablets for systemic effect. Rectal suppositories are used herein mean solid bodies for insertion into the rectum that melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration may be manufactured using the same pharmaceutically acceptable substance and by the same methods as for formulations for oral administration.

### Examples of Formulations:

The following are particular examples of oral, intravenous and tablet formulations that may optionally be used with compounds of the present invention. It is noted that these formulations may be varied depending on the particular compound being used and the indication for which the formulation is going to be used.

### Oral Formulation

| | | |
|---|---|---|
| Compound of the Present Invention | 10-100 | mg |
| Citric Acid Monohydrate | 105 | mg |
| Sodium Hydroxide | 18 | mg |
| Flavoring | | |
| Water | q.s. to 100 | mL |

### Intravenous Formulation

| Compound of the Present Invention | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |
| Water for Injection | q.s. to 1.0 | mL |

### Tablet Formulation

| | |
|---|---|
| Compound of the Present Invention | 1% |
| Microcrystalline Cellulose | 73% |
| Stearic Acid | 25% |
| Colloidal Silica | 1%. |

### Kits Comprising Mitotic Kinesin Inhibitors:

The invention is also directed to kits and other articles of manufacture for treating diseases associated with mitotic kinesins. It is noted that diseases are intended to cover all conditions for which the mitotic kinesin(s) possess activity that contributes to the pathology and/or symptomology of the condition.

In one embodiment, a kit is provided that comprises a composition comprising at least one mitotic kinesin inhibitor of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one mitotic kinesin inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

### Biological Testing:

The activity of compounds as KSP (HuEg5) inhibitors may be assayed in vitro, in vivo, or in a cell line. Further, compounds according to the present invention may be screened for activity against one or more kinesins. Provided below is the in vitro enzymatic KSP (HuEg5) ATPase activity assay for activity against KSP (HuEg5).

Purified KSP may be obtained as follows:

KSP (Eg5) DNA encoding residues 1-359 of the Kinesin motor domain sequence of the human enzyme may be amplified by PCR and cloned into the NcoI site of pET15b (Novagen), which incorporates a 6-histidine tag at the C-terminus. SEQ. I.D. No. 1 corresponds to residues 1-359 with the C-terminal 6-histidine tag and SEQ. I.D. No. 2 is the DNA sequence that was used to encode SEQ. I.D. No. 1.

Recombinant Protein incorporating the KSP (Eg5) construct may be generated by transformation of the plasmid into Tuner (DE3) E.coli cell. The expression of recombinant protein may be carried out by inducing the E.coli culture with IPTG and continuing to culture the E.coli cell for about 24 hours at 25° C.

Recombinant protein may be isolated from cellular extracts by passage over ProBond resin (Invitrogen), and dialysis against buffer containing 25 mM Tris pH 7.6, 50 mM NaCl, 0.25 mM TCEP. The purity of KSP (Eg5) proteins may be determined on denaturing SDS-PAGE gel. Purified KSP (Eg5) may then be concentrated to a final concentration of 3.4 mg/ml. The aliquots of protein may be stored at −78° C. in the above buffer or at −20° C. in the presence of glycerol (final concentration of glycerol at 50%).

The inhibitory properties of compounds relative to KSP may be determined using a clear 384-well plate format under the following reaction conditions: 20 mM K-PIPES pH 6.8, 2 mM MgCl₂, 1 mM EGTA, 1.5 mM KCl, 1 mM DTT, 0.01% Brij35, 25 μM ATP, 1 mM phosphoenolpyruvate (PEP), 0.4 mM NADH, 25 nM microtubules (MT), 1 μM Taxol, 30 units/ml rabbit muscle pyruvate kinase (PK), 30 units/ml rabbit muscle lactic dehydrogenase (LDH), and 2% DMSO. The production of ADP (via hydrolysis of ATP catalyzed by KSP) is coupled via pyruvate kinase and lactic dehydrogenase to the disappearance of NADH, which may be determined quantitatively by absorbance change at 340 nM using a UV-visible plate reader (Molecular Devices SpectraMax Plus).

The assay reaction may be initiated as follows: 5 μl of inhibitor (2-fold serial dilutions for 11 data points for each inhibitor) containing 10% DMSO was added to each well, followed by the addition of 10 μl of PK/LDH/MT mixture (2.5 times concentrated stock solution in reaction buffer containing ATP, PEP, PK, LDH, MT and Taxol). 10 μl of 30 nM KSP enzyme solution may be added to initiate the reaction (final enzyme concentration was 12 nM). Absorbance change at 340 nm of the resulting reaction mixtures may be measured after 60 minutes incubation at room temperature.

IC50 values may be calculated by non-linear curve fitting of the compound concentrations and absorbance to the standard IC50 equation. As a reference point for this assay, Monastrol and HR22C16 showed IC50s of 45 μM and 1.5 μM, respectively, for the KSP (HuEg5) assay.

### Examples

### Synthetic Scheme For KSP Inhibitors:

KSP inhibitors according to the present invention may be synthesized according to a variety of reaction schemes. Illustrative schemes are provided herein. Other reaction schemes could be readily devised by those skilled in the art.

Various substituted benzimidazole analogues may be prepared using methods generally known in organic synthesis. One method for the preparation of the KSP inhibitors is shown in Scheme I. Various derivatives of 1-fluoro-2-nitro-benzene may be reacted with an amine to faun a 2-nitro-aniline derivative 1. Reduction of the nitro benzene compound provides the ortho amino aniline derivative 2. Reduction of the nitro benzene compound may be accomplished using standard conditions known in the art, including hydrogen and metal catalyst such as Pd on carbon. Reduction may also be performed via transfer hydrogenation methodology utilizing reagents such as hydrazine and Raney-Nickel. Condensation with a protected amino acid derivative afford the N-acyl aniline derivative 3, which may be cyclized to afford the benzimidazole derivative 4. For example, the aniline 2 may be coupled with a protected amino acid to form the N-acylated aniline derivative 3. Coupling of the aniline 2 may be performed using various coupling agents known in the art for coupling amines and acids, including for example EDCl/HOBT in a polar organic solvent such as DMF. Cyclization of the N-acyl aniline 3 using an acid, such as acetic acid, forms the 2-substituted benzimidazole 4. Reductive amination provides the alkylamino benzimidazole 6. Acylation of the alkylamino benzimidazole 6 provides the benzimidazole analogues 7. Alternatively, alkylamino benzimidazole 6 can be alkylated to provide analogues 8.

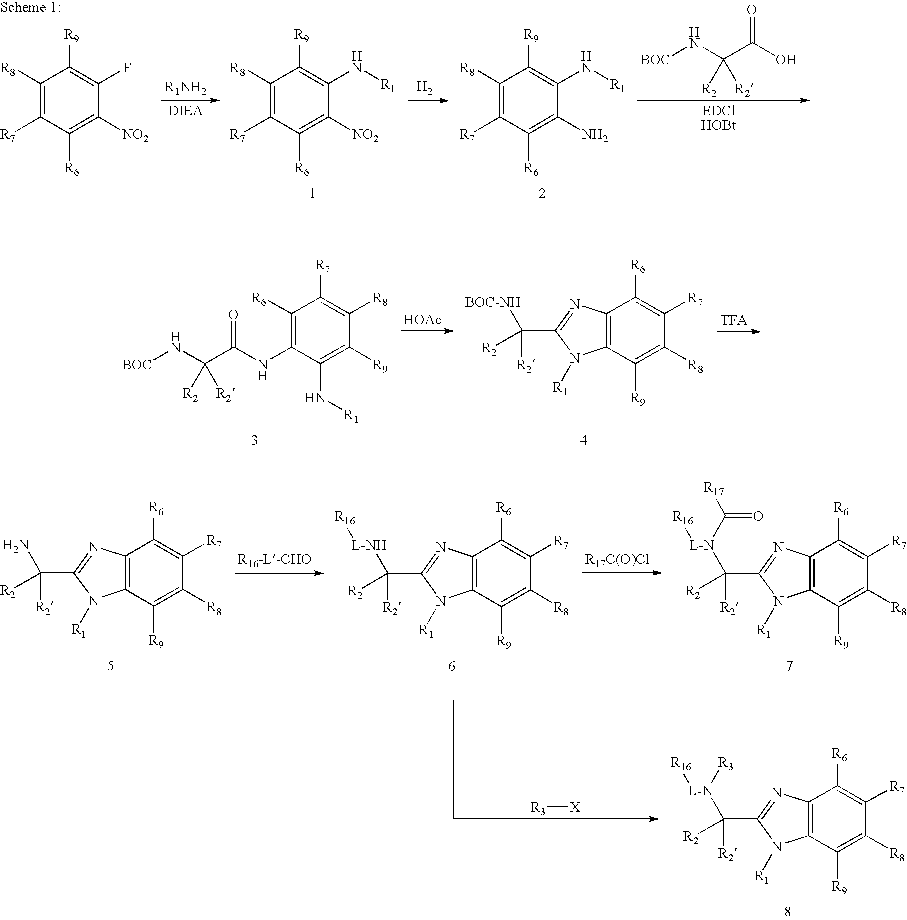

For example, the above reaction scheme, variations thereof, and other reaction schemes readily devised by those skilled in the art can be used to prepare the following:

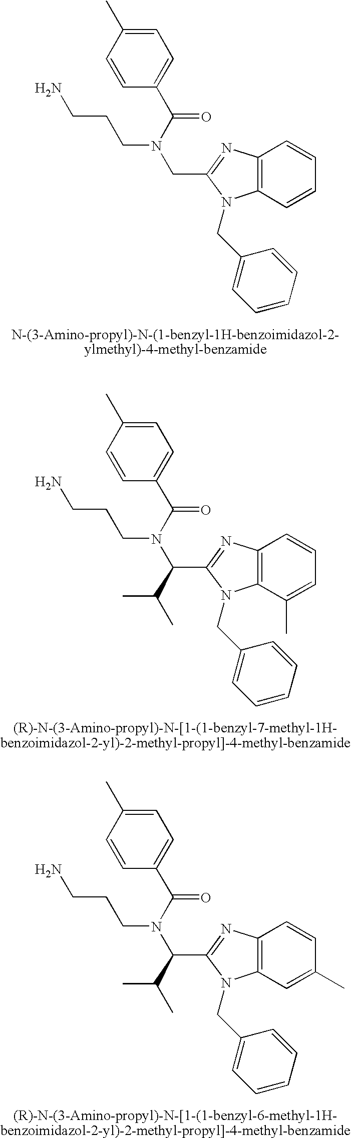

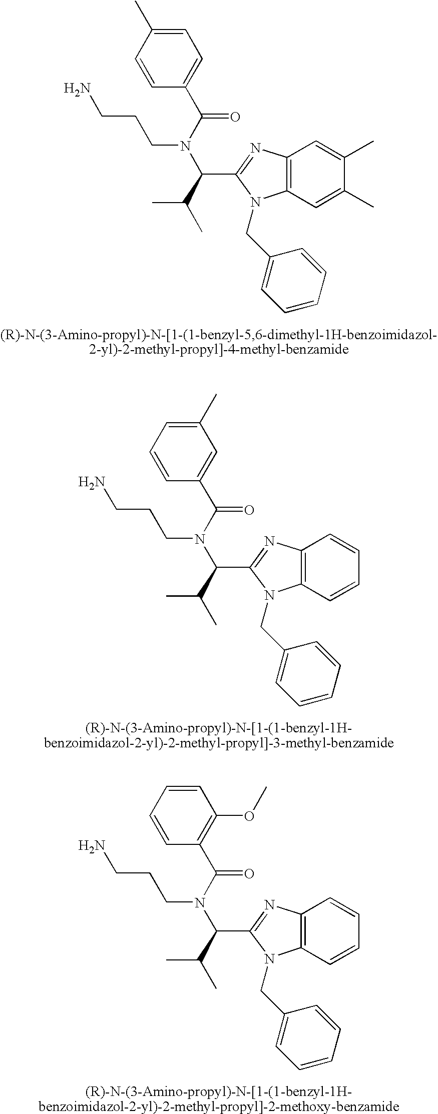

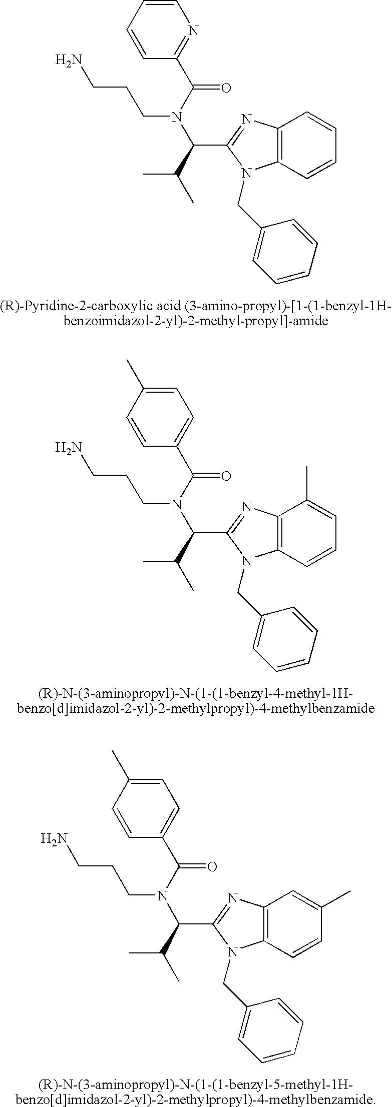

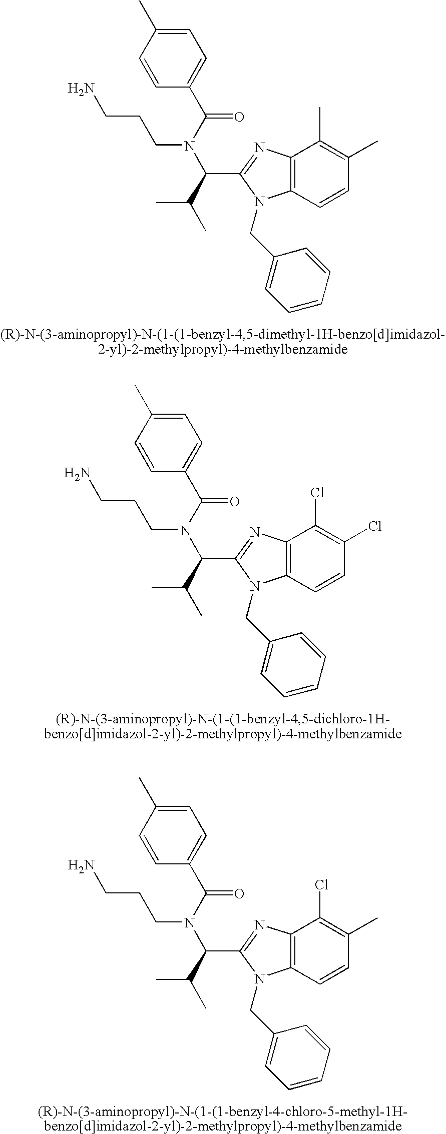

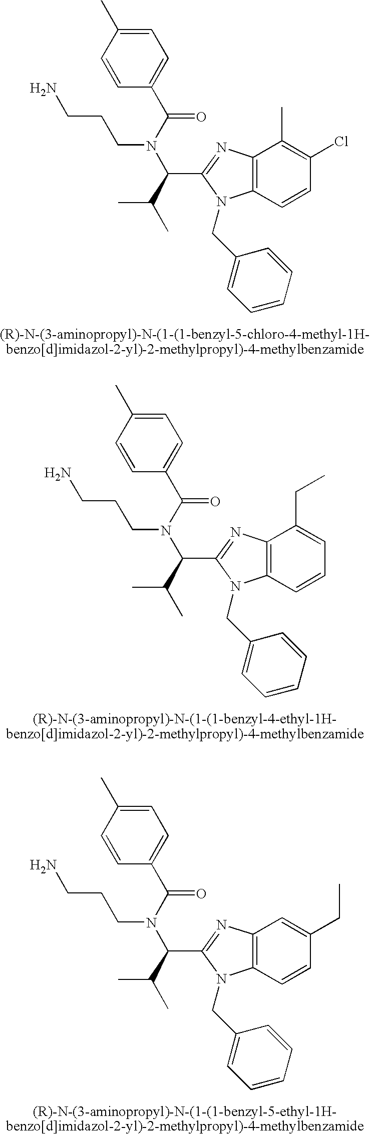

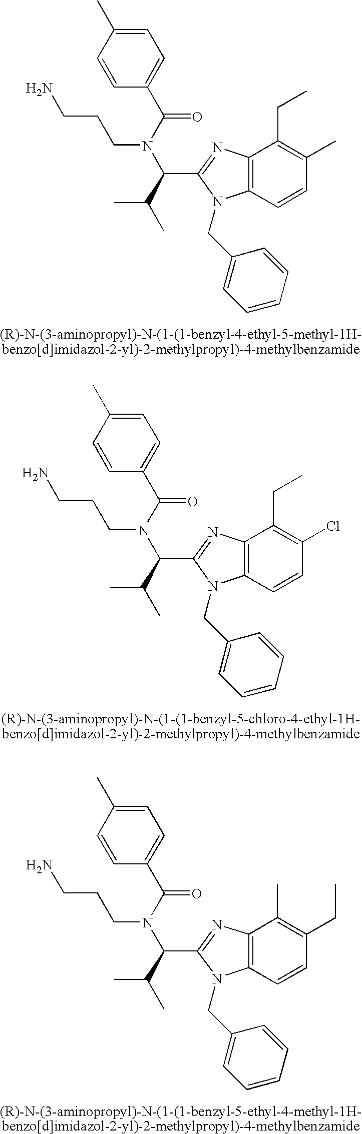

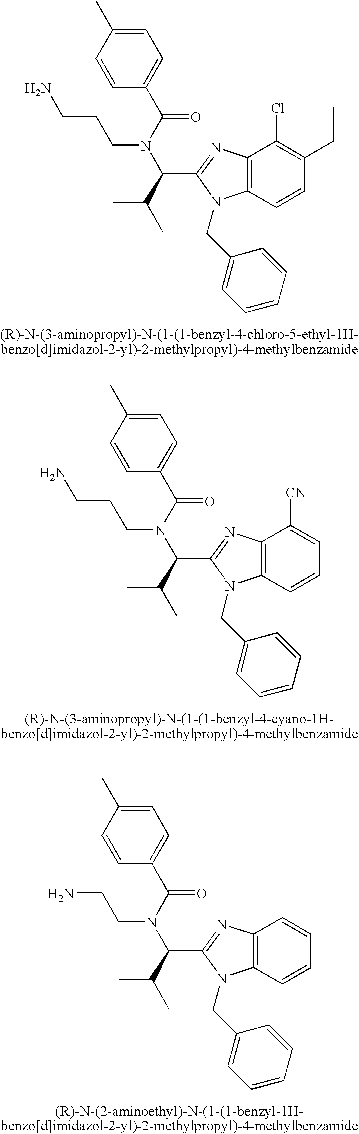

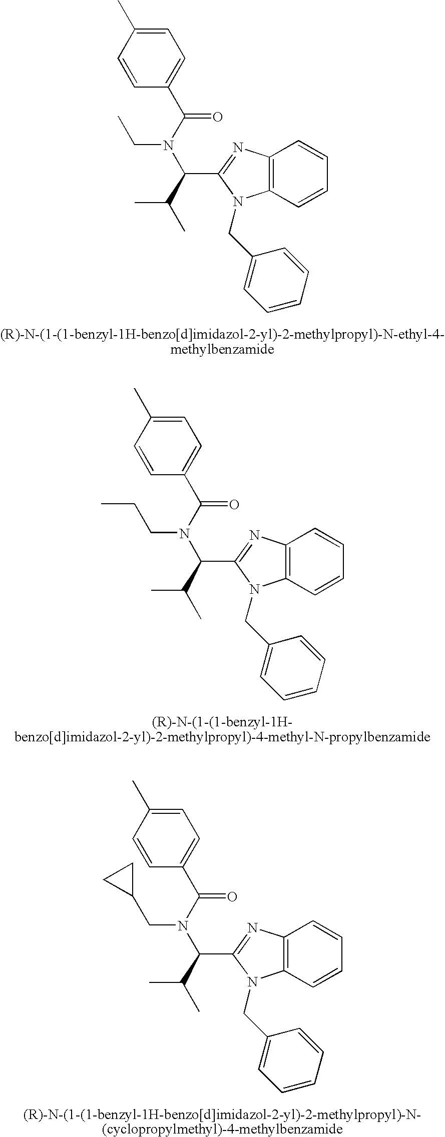

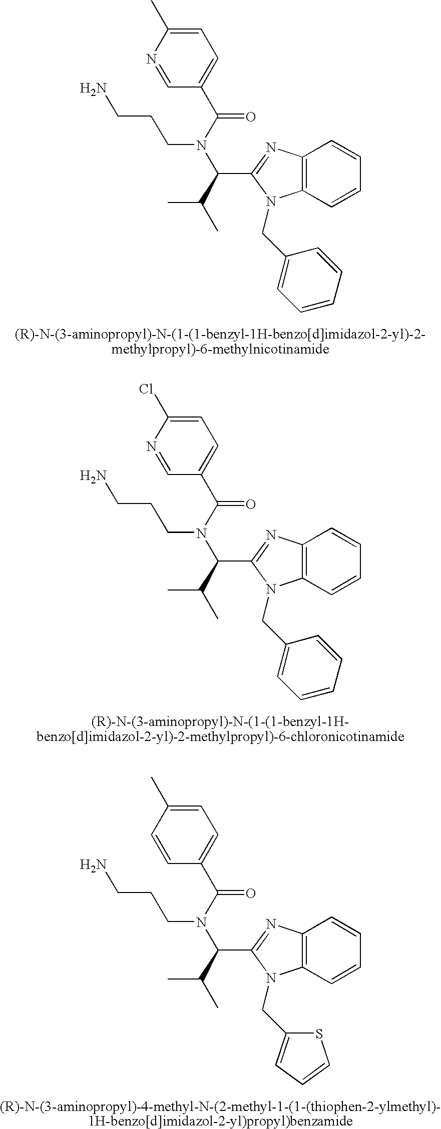

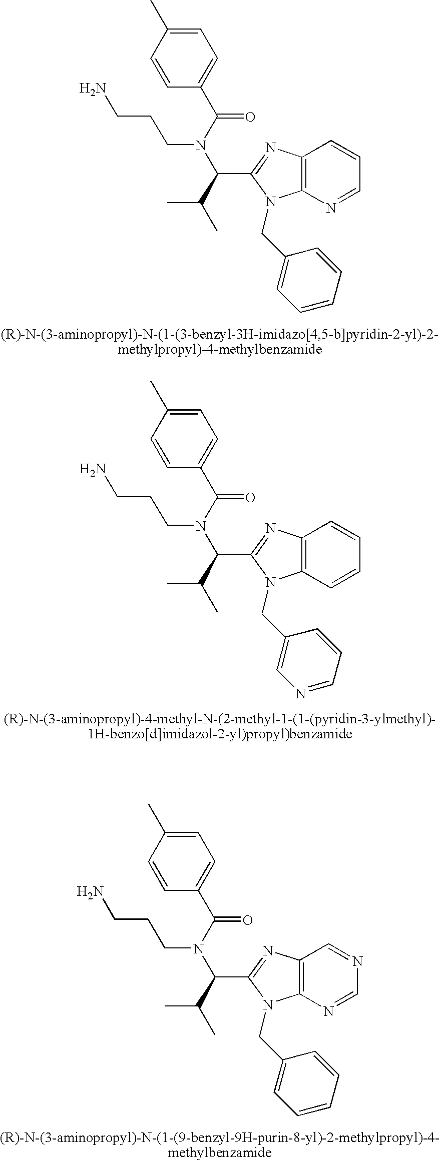

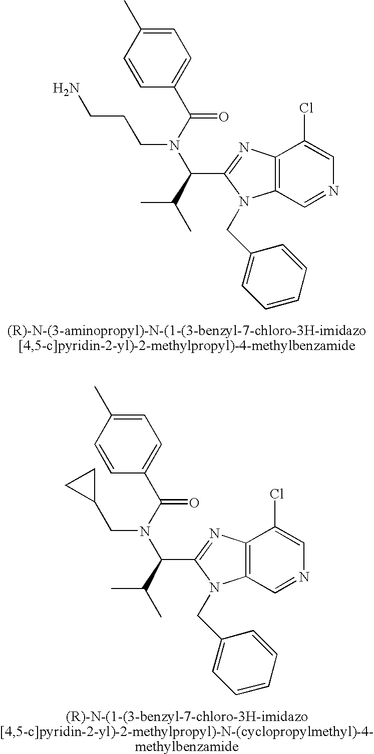

As can be seen from the above reaction scheme, a wide variety of different KSP inhibitors can be synthesized. It is noted that the invention is not intended to be limited to the particular compounds provided in the examples. Rather, a wide variety of other compounds according to the present invention having KSP inhibitory activity may be synthesized by the reaction scheme provided as well as other reaction schemes that may be devised by one of ordinary skill in the art in view of the present teachings.

The compounds of the invention illustrated below can be prepared by the synthetic methods described hereinabove by substituting the appropriate 1-fluoro-2-nitro-benzene, amine, carboxylic acid, carboxylic acid chloride, and aldehyde for the corresponding reagents utilized in the above example.

### Examples of Inhibitors According to the Present Invention:

Provided in this example are particular compounds that have been found to have KSP activity based on the assay provided herein. It is noted that these compounds may also have activity relative to other Kinesins. It is also noted that these compounds are intended to illustrate various KSP inhibitors according to the present invention and the present invention is not intended to be limited to these compounds:

### Example 1

### (R)—N-(3-amino-propyl)-N-[1-(1-benzyl-1H-benzoimidazol-2-yl)-2-methyl-propyl]-4-methyl-benzamide

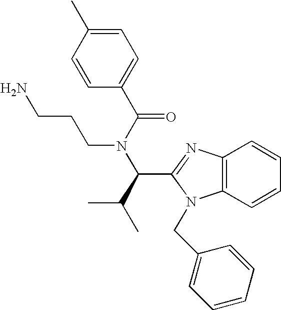

### Benzyl-(2-nitro-phenyl)-amine

To a solution of 1-fluoro-2-nitro-benzene (7.1 mmol) in DMF (2.5 mL) was added benzylamine (7.3 mmol) and DIEA (7.8 mmol). The reaction was stirred at 60° C. for 2 hrs, cooled to ambient temperature, and poured into H₂O (50 mL). The aqueous layer was extracted with EtOAc (2×25 mL). The combined organic layers were washed with brine (1×50 mL), dried over MgSO₄, and concentrated to dryness to provide benzyl-(2-nitro-phenyl)-amine.

### N-Benzyl-benzene-1,2-diamine

To a solution of benzyl-(2-nitro-phenyl)-amine and RaNi (0.7 mmol) in MeOH (20 mL) was added hydrazine monohydrate (14.0 mmol) portion-wise over 30 min at ambient temperature. The reaction was stirred for an additional 15 min at ambient temperature and then filtered over celite. The filtrate was concentrated to dryness and reconstituted in EtOAc (50 mL). The organic solution was washed with H₂O (1×50 mL) and brine (1×50 mL), and then it was dried over MgSO₄ and concentrated to dryness to provide N-benzyl-benzene-1,2-diamine.

### (R)-[1-(2-Benzylamino-phenylcarbamoyl)-2-methyl-propyl]-carbamic acid tert-butyl ester

To a solution of the appropriate BOC-D-valine (9.8 mmol), EDCI (13.0 mmol), and HOBt (13.0 mmol) in anhydrous DMF (10 mL) was added N-benzyl-benzene-1,2-diamine and DIEA (19.5 mmol). The reaction was stirred at ambient temperature 2 hrs. The reaction was then poured into H₂O (100 mL) and extracted with EtOAc (2×25 mL). The combined organic layers were washed with H₂O (1×50 mL) and brine (1×50 mL), dried over MgSO₄, and concentrated to dryness. The resulting residue was purified via column chromatography with hexanes/EtOAc (9:1) to provide (R)-[1-(2-benzylamino-phenylcarbamoyl)-2-methyl-propyl]-carbamic acid tert-butyl ester.

### (R)-[1-(1-Benzyl-1H-benzoimidazol-2-yl)-2-methyl-propyl]-carbamic acid tert-butyl ester

A solution of (R)-[1-(2-benzylamino-phenylcarbamoyl)-2-methyl-propyl]-carbamic acid tert-butyl ester in EtOH/AcOH (9:1, 5 mL) was heated at 90° C. for 36 hrs. The reaction was cooled to ambient temperature and allowed to stand for 24 hrs. The resulting crystals were filtered, washed with ice cold EtOH (2×5 mL), and dried to provide (R)-[1-(1-benzyl-1H-benzoimidazol-2-yl)-2-methyl-propyl]-carbamic acid tert-butyl ester.

### (R)-1-(1-Benzyl-1H-benzoimidazol-2-yl)-2-methyl-propylamine

A solution of (R)-[1-(1-benzyl-1H-benzoimidazol-2-yl)-2-methyl-propyl]-carbamic acid tert-butyl ester in DCM/TFA/Et₃Si (50:49:1, 5 mL) was stirred at ambient temperature for 3 hrs. The reaction was concentrated to dryness and the residue was reconstituted in DCM (25 mL). The organic solution was washed with saturated NaHCO₃ (2×25 mL) and brine (1×25 mL), dried over MgSO₄, and concentrated to dryness to provide (R)-1- (1-benzyl-1H-benzoimidazol-2-yl)-2-methyl-propyl amine.

### (R)-(3-[1-(1-Benzyl-1H-benzoimidazol-2-yl)-2-methyl-propylamino]-propyl]-carbamic acid tert-butyl ester

To a mixture of molecular sieves (4A, 500 mg), (R)-1-(1-benzyl-1H-benzoimidazol-2-yl)-2-methyl-propylamine (5.0 mmol), NaOAc (10.0 mmol) and MeOH (5 mL) was added (3-oxo-propyl)-carbamic acid tert-butyl ester (5.5 mmol) and then NaCNBH₃ (10.0 mmol) at ambient temperature under an inert atmosphere. The reaction was stirred at ambient temperature for 2 hours and then poured into saturated NaHCO₃ (50 mL). The aqueous mixture was extracted with DCM (1×50 mL), and the organic phase was washed with brine, dried over MgSO₄, and concentrated to dryness. The resulting residue was purified by column chromatography with hexanes/EtOAc (1:1) to provide (R)-{3-[1-(1-benzyl-1H-benzoimidazol-2-yl)-2-methyl-propylamino]-propyl}-carbamic acid tert-butyl ester.

(R)—N-(3-amino-propyl)-N-[1-(1-benzyl-1H-benzoimidazol-2-yl)-2-methyl-propyl]-4-methyl-benzamide

To a solution of (R)-{3-[1-(1-benzyl-1H-benzoimidazol-2-yl)-2-methyl-propylamino]-propyl}-carbamic acid tert-butyl ester (4.0 mmol) in DCM (2 mL) was added TEA (4.4 mmol) and 4-methyl-benzoyl chloride (4.0 mmol) at ambient temperature. The reaction was allowed to stir for 30 min at ambient temperature and then treated with TFA (2.0 mL) and allowed to stir for an additional 30 min. The reaction was then concentrated to dryness and the residue was reconstituted in DMF(2.0 mL). The resulting solution was purified via preparative LCMS to provide (R)—N-(3-amino-propyl)-N-[1-(1-benzyl-1H-benzoimidazol-2-yl)-2-methyl-propyl]-4-methyl-benzamide. ¹H NMR (400 MHz, DMSO-D₆) δ ppm 0.55 (m, 1H) 0.76 (m, 3H) 1.04 (m, 4H) 1.96 m, 2H) 2.30 (s, 3 H) 2.84 (m, 1H) 3.22 (m, 2H) 5.60 (dd, 2H). 5.81 (d, 1H) 7.08 (m, 4H) 7.16-7.25 (band, 4H) 7.32 (m, 3H) 7.50 (m, 1H) 7.72 (m, 1H). ESI-MS: m/z 455.2 (M+H)⁺.

### Example 2

### (R)—N-(3-Amino-propyl)-N-[1-(1-benzyl-1H-benzoimidazol-2-yl)-ethyl]-4-methyl-benzamide

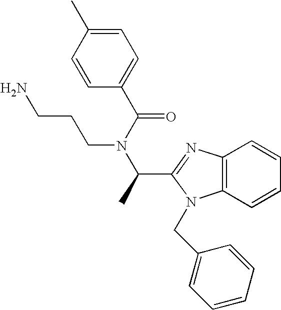

Example 2 was prepared analogously to Example 1. ESI-MS: m/z 427.2 (M+H)⁺.

### Example 3

### (R)—N-(3-Amino-propyl)-N-[1-(1-benzyl-1H-benzoimidazol-2-yl)-propyl]-4-methyl-benzamide

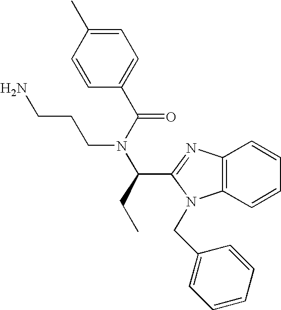

Example 3 was prepared analogously to Example 1. ESI-MS: m/z 441.2 (M+H)⁺.

### Example 4

### (R)—N-(3-Amino-propyl)-N-[1-(1-benzyl-7-chloro-1H-benzoimidazol-2-yl)-2-methyl-propyl]-4-methyl-benzamide

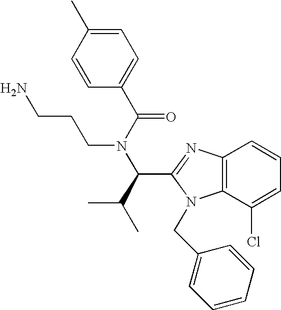

Example 4 was prepared analogously to Example 1. ESI-MS: m/z 489.2 (M+H)⁺.

### Example 5

### (R)—N-(3-Amino-propyl)-N-[1-(1-benzyl-6-chloro-1H-benzoimidazol-2-yl)-2-methyl-propyl]-4-methyl-benzamide

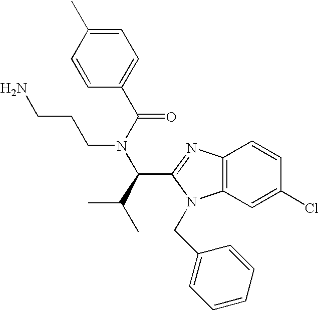

Example 5 was prepared analogously to Example 1. ESI-MS: m/z 489.2 (M+H)⁺.

### Example 6

### (R)—N-(3-Amino-propyl)-N-[1-(1-benzyl-5-chloro-1H-benzoimidazol-2-yl)-2-methyl-propyl]-4-methyl-benzamide

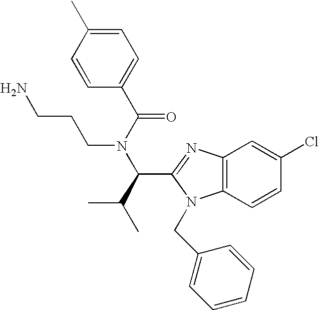

Example 6 was prepared analogously to Example 1. ESI-MS: m/z 489.2 (M+H)⁺.

### Example 7

### (R)—N-(3-Amino-propyl)-N-[1-(1-benzyl-5,6-dichloro-1H-benzoimidazol-2-yl)-2-methyl-propyl]-4-methyl-benzamide

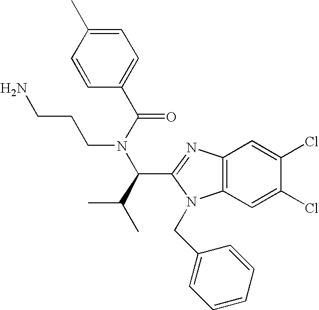

Example 7 was prepared analogously to Example 1. ESI-MS: m/z 523.2 (M+H)⁺.

### Example 8

### (R)—N-(3-Amino-propyl)-N-[1-(1-benzyl-1H-benzoimidazol-2-yl)-2-methyl-propyl]-2-methyl-benzamide

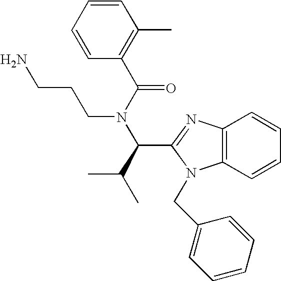

Example 8 was prepared analogously to Example 1. ¹H NMR (400 MHz, DMSO-D₆) δ ppm 0.56 (m, 1H) 0.73 (m, 3H) 1.08 (m, 4H) 1.96 (m, 2H) 2.19 (s, 3H) 2.85 (m, 1H) 3.16 (m, 2H) 5.49-5.73 (dd, 2H) 5.83 (d, 1H) 7.13 (m, 2H) 7.21-7.38 (band, 9H) 7.53 (m, 1H) 7.71 (m, 1H). ESI-MS: m/z 455.2 (M +H)⁺.

### Example 9

### (R)—N-(3-Amino-propyl)-N-[1-(1-benzyl-1H-benzoimidazol-2-yl)-2-methyl-propyl]-4-chloro-benzamide

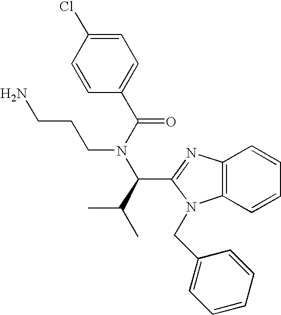

Example 9 was prepared analogously to Example 1. ¹H NMR (400 MHz, DMSO-D₆) δ ppm 0.63 (m, 1H) 0.74 (m, 3H) 1.02 (m, 3H) 1.16 (m, 1H) 2.04 (m, 2H) 2.83 (m, 1H) 3.22 (m, 2H) 5.59 (dd, 2H) 5.79 (d, 1H) 7.08 (m, 2H) 7.14-7.38 (band, 7 H) 7.44 (m, 2H) 7.52 (m, 1H) 7.72 (m, 1H). ESI-MS: m/z 475.2 (M +H)⁺.

### Example 10

### (R)—N-(3-Amino-propyl)-N-[1-(1-benzyl-1H-benzoimidazol-2-yl)-2-methyl-propyl]-3-chloro-benzamide

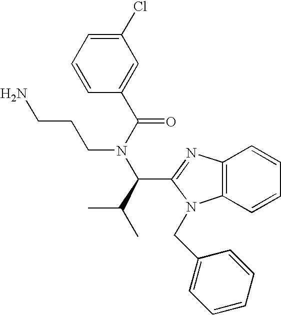

Example 10 was prepared analogously to Example 1. ¹H NMR (400 MHz, DMSO-D₆) δ ppm 0.66 (m, 1H) 0.77 (m, 3H) 1.03 (m, 3H) 1.14 (m, 1H) 2.04 (m, 2H) 2.83 (m, 1H) 3.20 (m, 2H) 5.60 (dd, 2H) 5.80 (d, 1H) 7.06-7.15 (band, 4H) 7.22-7.42 (band, 6H) 7.49 (m, 2H) 7.73 (m, 1H). ESI-MS: m/z 475.2 (M +H)⁺.

### Example 11

### (R)—N-(3-Amino-propyl)-N-[1-(1-benzyl-1H-benzoimidazol-2-yl)-2-methyl-propyl]-2-chloro-benzamide

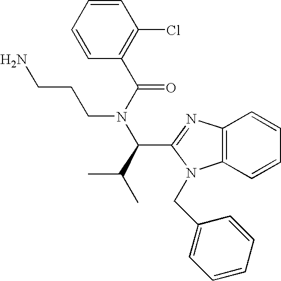

Example 11 was prepared analogously to Example 1. ¹H NMR (400 MHz, DMSO-D₆) δ ppm 0.58 (m, 1H) 0.75 (m, 3H) 1.06 (m, 3H) 1.22 (m, 1H) 2.00 (m, 2H) 2.85 (m, 1H) 3.09 (m, 1H) 3.22 (m, 1H) 5.51 (tt, 2H) 5.79 (m, 1H) 7.13 (m, 2H) 7.21-7.58 (band, 10H) 7.73 (m, 1H). m/z 475.2 (M+H)⁺.

### Example 12

### (R)—N-(3-Amino-propyl)-N-[1-(1-benzyl-1H-benzoimidazol-2-yl)-2-methyl-propyl]-4-methoxy-benzamide

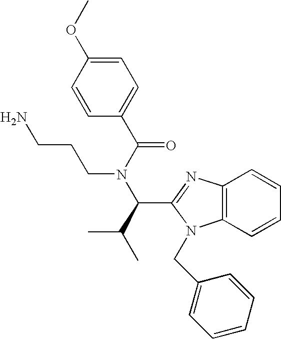

Example 12 was prepared analogously to Example 1. ¹H NMR (400 MHz, DMSO-D₆) δ ppm 0.58 (m, 1H) 0.75 (m, 3H) 1.06 (m, 4H) 2.01 (m, 1H) 2.82 (m, 1H) 3.27 (m, 2H) 3.78 (s, 3H) 5.60 (dd, 2H) 5.81 (d, 1H) 6.93 (m, 2H) 7.07-7.35 (band, 9H) 7.48 (m, 1H) 7.71 (m, 1H). ESI-MS: m/z 471.2 (M+H)⁺.

### Example 13

### (R)—N-(3-Amino-propyl)-N-[1-(1-benzyl-1H-benzoimidazol-2-yl)-2-methyl-propyl]-3-methoxy-benzamide

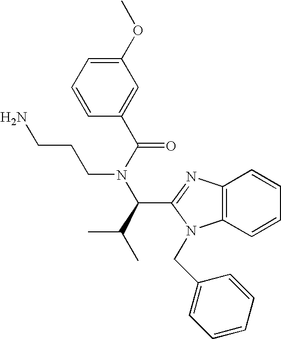

Example 13 was prepared analogously to Example 1. ESI-MS: m/z 471.2 (M+H)⁺.

### Example 14

### (R)—N-(3-Amino-propyl)-N-[1-(1-benzyl-1H-benzoimidazol-2-yl)-2-methyl-propyl]-4-cyano-benzamide

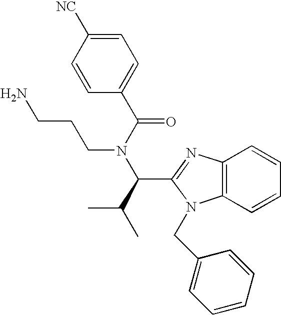

Example 14 was prepared analogously to Example 1. ¹H NMR (400 MHz, DMSO-D₆) δ ppm 0.56 (m, 1H) 0.75 (m, 3H) 1.04 (s, 4H) 2.00 (m, 2H) 2.84 (m, 1H) 3.20 (m, 2H) 5.59 (dd, 2 11) 5.78 (d, 1H) 7.09 (m, 2H) 7.22-7.40 (band, 7H) 7.53(m, 1H) 7.72 (m, 1H) 7.87 (m, 2H). ESI-MS: m/z 466.2 (M+H)⁺.

### Example 15

### (R)—N-(3-Amino-propyl)-N-[1-(1-benzyl-1H-benzoimidazol-2-yl)-2-methyl-propyl]-3-cyano-benzamide

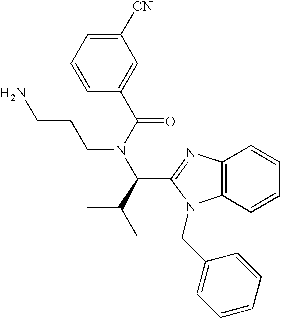

Example 15 was prepared analogously to Example 1. ¹H NMR (400 MHz, DMSO-D₆) δ ppm 0.64 (m, 1H) 0.78 (m, 3H) 1.07 (m, 4H) 2.04 (m, 2H) 2.84 (m, 1H) 3.21 (m, 2H) 5.54 (m, 1H) 5.66 (m, 1H) 5.79 (m, 1H) 7.07 (m, 2H) 7.23-7.37 (band, 5H) 7.44-7.61 (band, 4H) 7.73 (m, 1H) 7.87 (m, 1H). ESI-MS: m/z 466.2 (M+H)⁺.

### Example 16

### (R)—N-(3-Amino-propyl)-N-[1-(1-benzyl-1H-benzoimidazol-2-yl)-2-methyl-propyl]-nicotinamide

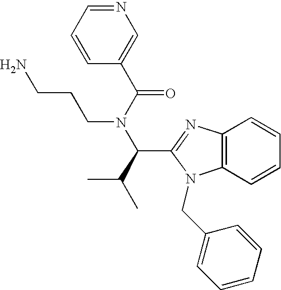

Example 16 was prepared analogously to Example 1. ¹H NMR (400 MHz, DMSO-D₆) δ ppm 0.56 (m, 1H) 0.78 (m, 3 11) 1.04 (m, 4H) 1.97 (m, 2H) 2.83 (m, 1H) 3.24 (m, 2H) 5.61 (dd, 2H) 5.81 (d, 1H) 7.08 (d, 2H) 7.23-7.36 (band, 5H) 7.41 (m, 1H) 7.53 (m, 1H) 7.60 (m, 1H) 7.72 (m, 1H) 8.34 (m, 1H) 8.60 (m, 1H). ESI-MS: m/z 442.2 (M+H)⁺.

### Example 17

### (R)-Furan-2-carboxylic acid (3-amino-propyl)-[1-(1-benzyl-1H-benzoimidazol-2-yl)-2-methyl-propyl]-amide

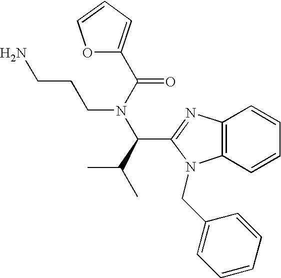

Example 17 was prepared analogously to Example 1. ¹H NMR (400 MHz, DMSO-D₆) δ ppm 0.68 (m, 4H) 0.88 (d, 3H) 1.28 (m, 1H) 2.26 (m, 2H) 2.81 (m, 1H) 3.46 (m, 1H) 3.75 (m, 1H) 5.52 (dd, 2H) 5.78 (d, 1H) 6.62 (s, 1H) 7.09 (m, 3H) 7.20-7.30 (band, 5H) 7.51 (m, 1H) 7.74 (m, 1H) 7.84 (m, 1H). ESI-MS: m/z 431.2 (M+H)⁺.

### Example 18

### (R)-Thiophene-2-carboxylic acid (3-amino-propyl)-[1-(1-benzyl-1H-benzo-imidazol-2-yl)-2-methyl-propyl]-amide

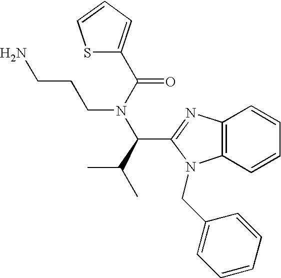

Example 18 was prepared analogously to Example 1. ¹H NMR (400 MHz, DMSO-D₆) δ ppm 0.71 (m, 4H) 0.95 (m, 3H) 1.31 (s, 1H) 2.20 (s, 2H) 2.83 (m, 1H) 3.49 (m, 1H) 3.68 (m, 1H) 5.54 (dd, 2H) 5.60 (d, 1H) 7.07 (m, 3H) 7.20-7.31 (band, 5H) 7.50 (m, 2H) 7.74 (m, 2H). ESI-MS: m/z 447.2 (M+H)⁺.

### Example 19

### (R)—N-(3-Amino-propyl)-N-[1-(1-benzyl-1H-benzoimidazol-2-yl)-2-methyl-propyl]-2-phenyl-acetamide

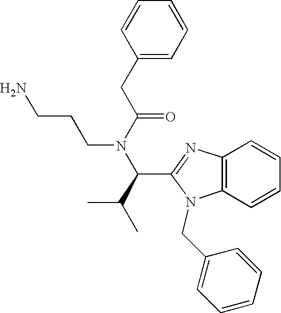

Example 19 was prepared analogously to Example 1. ¹H NMR (400 MHz, DMSO-D₆δ ppm 0.65 (d, 3H) 0.77 (d, 3H) 1.40 (m, 1H) 2.37 (t, 2H) 2.71 (m, 1H) 3.25 (m, 1H) 3.50 (m, 2H) 3.78 (d, 1H) 5.43 (dd, 2H) 5.67 (d, 1H) 7.04 (d, 2H) 7.18-7.33 (band, 10H) 7.49 (m, 1H) 7.70 (m, 1H). ESI-MS: m/z 455.2 (M+H)⁺.

### Example 20

### (R)—N-(3-Amino-propyl)-N-[1-(1-benzyl-1H-benzoimidazol-2-yl)-2-methyl-propyl]-3-phenyl-propionamide

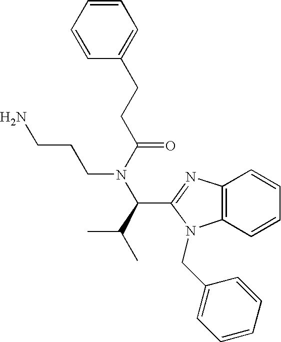

Example 20 was prepared analogously to Example 1. ¹H NMR (400 MHz, DMSO-D₆) δ ppm 0.63 (m, 1H) 0.70 (d, 3H) 0.77 (d, 3H) 1.29 (m, 1H) 2.35 (m, 3H) 2.63-2.86 (band, 5H) 3.14 (m, 1H) 5.39 (dd, 2H) 5.69 (d, 1H) 7.02 (d, 2H) 7.14-7.34 (band, 10H) 7.47 (m, 1H) 7.71 (m, 1H). ESI-MS: m/z 469.2 (M+H)⁺.

### Example 21

### (R)—N-(3-Amino-propyl)-N-[1-(1-benzyl-1H-benzoimidazol-2-yl)-2-methyl-propyl]-4-ethyl-benzamide

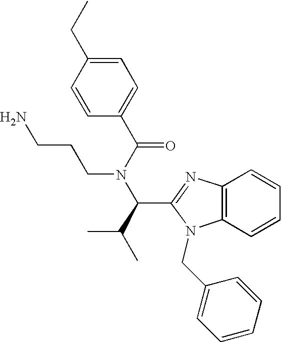

Example 21 was prepared analogously to Example 1. ¹H NMR (400 MHz, DMSO-D₆) δ ppm 0.75 (m, 4H) 1.02 (d, 3H) 1.22 (m, 1H) 2.01 (t, 3H) 2.62 (m, 2.83 (m, 1H) 3.21 (m, 3H) 5.61 (dd, 2H) 5.82 (d, 1H) 7.07 (m, 4H) 7.18-7.35 (band, 5H) 7.50 (m, 1H) 7.71 (m, 1H). ESI-MS: m/z 469.2 (M+H)⁺.

### Example 22

### (R)—N-(3-Amino-propyl)-4-methyl-N-[2-methyl-1-(1-methyl-1H-benzoimidazol-2-yl)-propyl]-benzamide

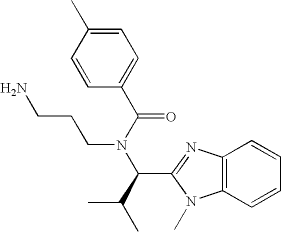

Example 22 was prepared analogously to Example 1. ¹H NMR (400 MHz, DMSO-D₆) δ ppm 0.96 (m, 4H) 1.08 (m, 3H) 1.84 (m, 2H) 2.32 (s, 3H) 3.14 (m, 2H) 3.28 (s, 1H) 3.82 (s, 3H) 5.80 (m, 1H) 7.18 -7.29 (band, 6H) 7.56 (m, 1H) 7.66 (m, 1H). ESI-MS: m/z 379.2 (M+H)⁺.

### Example 23

### (S)—N-(3-Amino-propyl)-N-[1-(1-benzyl-1H-benzoimidazol-2-yl)-2-methyl-propyl]-4-methyl-benzamide

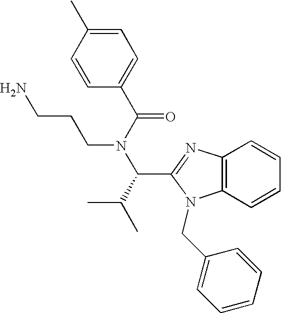

Example 23 was prepared analogously to Example 1. ¹H NMR (400 MHz, DMSO-D₆) δ ppm 0.52 (m, 1H) 0.78 (d, 3H) 1.04 (m, 4H) 1.94 (t, 2H) 2.31 (s, 3H) 2.85 (m, 1H) 3.22 (m, 2H) 5.61 (dd, 2H) 5.82 (d, 1H) 7.09(m, 4H) 7.18-7.37 (band, 7H) 7.50 (m, 1H) 7.72 (m, 1H). ESI-MS: m/z 455.2 (M+H)⁺.

### Example 24

### (R)—N-(3-aminopropyl)-N-(1-(1-benzyl-4-chloro-1H-benzo[d]imidazol-2-yl)-2-methylpropyl)-4-methylbenzamide

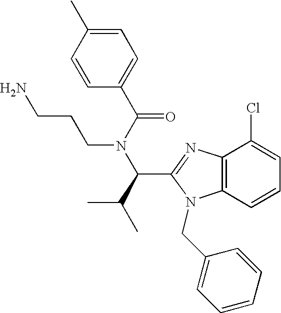

Example 24 was prepared analogously to Example 1. ESI-MS: m/z 489.2 (M+H)⁺.

### Example 25

### (R)—N-(3-aminopropyl)-N-(1-(1-benzyl-1H-benzo[d]imidazol-2-yl)-2-methylpropyl)-5-ethylthiophene-3-carboxamide

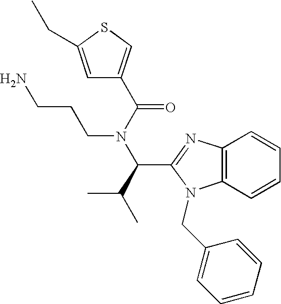

Example 25 was prepared analogously to Example 1. ¹H NMR (400 MHz, DMSO-D₆) δ ppm 0.61 (m, 1H) 0.78 (d, 3H) 1.00 (d, 3H) 1.12 (m, 1H) 1.22 (t, 4H) 2.06 (t, 2H) 2.46 (s, 1H) 2.80 (m, 3H) 5.58 (dd, 2H) 5.81 (d, 1H) 6.76 (s, 1) 7.09 (d, 2H) 7.23-7.39 (band, 5H) 7.44-7.53 (band, 2H) 7.73 (s, 1H). ESI-MS: m/z 475.2 M+H)⁺.

### Example 26

### (R)—N-(3-aminopropyl)-N-(1-(1-benzyl-1H-benzo[d]imidazol-2-yl)-2-methylpropyl)-4-bromo-5-ethylthiophene-2-carboxamide

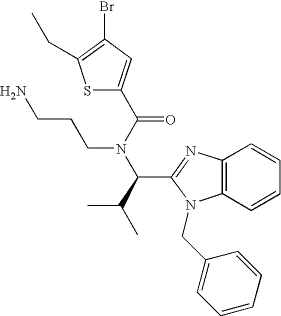

Example 26 was prepared analogously to Example 1. 1H NMR (400 MHz, DMSO-D6) δ ppm 0.72 (m, 4H) 0.92 (d, 3H) 1.21 (m, 3H) 1.34 (m, 1H) 2.25 (m, 2H) 2.75 (m, 3H) 3.46 (m, 1H) 3.69 (m, 1H) 5.51 (dd, 2H) 5.77 (d, 1H) 7.04 (m, 2H) 7.19-7.31 (band, 5H) 7.52 (m, 2H) 7.73 (d, 1H). ESI-MS: m/z 553.6 (M+H)⁺.

### Example 27

### (R)—N-(3-aminopropyl)-N-(1-(1-benzyl-1H-benzo[d]imidazol-2-yl)-2-methylpropyl)-4-bromo-5-propylthiophene-2-carboxamide

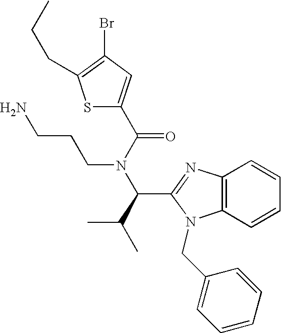

Example 27 was prepared analogously to Example 1. 1H NMR (400 MHz, DMSO-D6) δ ppm 0.74 (m, 4H) 0.93 (m, 5H) 1.31 (m, 1H) 1.62 (m, 2H) 2.26 (m, 2H) 2.72 (t, 3H) 2.83 (m, 1H) 3.48 (m, 1H) 3.73 (m, 1H) 5.50 (dd, 2H) 5.81 (m, 2H) 7.04 (s, 2H) 7.18-7.32 (band, 5H) 7.53 (m, 2H) 7.74 (m 1H). ESI-MS: m/z 566.6 (M+H)⁺.

### Example 28

### (R)—N-(3-aminopropyl)-N-(1-(1-benzyl-1H-benzo[d]imidazol-2-yl)-2-methylpropyl)-5-methylthiophene-2-carboxamide

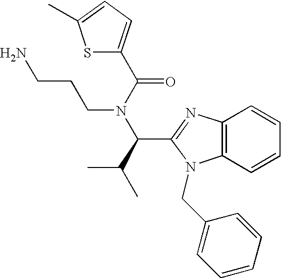

Example 28 was prepared analogously to Example 1. ¹H NMR (400 MHz, DMSO-D₆) δ ppm 0.65 (m, 1H) 0.73 (d, 3H) 0.95 (m, 4H) 2.23 (m, 2H) 2.47 (s, 3H) 2.85 (m, 1H) 3.51 (m, 1H) 3.71 (m, 1H) 5.54 (dd, 2H) 5.81 (d, 1H) 6.82 (m, 2H) 7.09 (m, 2H) 7.23-7.40 (band, 4H) 7.51 (m, 2H) 7.74 (m, 1H). ESI-MS: m/z 461.2 (M+H)⁺.

### Example 29

### (R)—N-(3-aminopropyl)-N-(1-(1-benzyl-1H-benzo[d]imidazol-2-yl)-2-methylpropyl)-5-isopropylthiophene-3-carboxamide

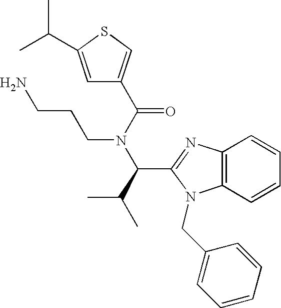

Example 29 was prepared analogously to Example 1. ¹H NMR (400 MHz, DMSO-D₆) δ ppm 0.79 (d, 3H) 0.85 (m, 1H) 0.99 (d, 3H) 1.25 (m, 7H) 2.07 (t, 2H) 2.46 (s, 2H) 2.82 (m, 1H) 3.13 (m, 1H) 5.58 (dd, 2H) 5.78 (d, 1H) 6.75 (s, 1H) 7.09 (d, 2H) 7.21-7.39 (band, 4H) 7.49 (m, 2H) 7.73 (m, 1H). ESI-MS: m/z 489.2 (M+H)⁺.

### Example 30

### (R)—N-(3-aminopropyl)-N-(1-(1-benzyl-1H-benzo[d]imidazol-2-yl)-2-methylpropyl)-5-(methylthio)thiophene-2-carboxamide

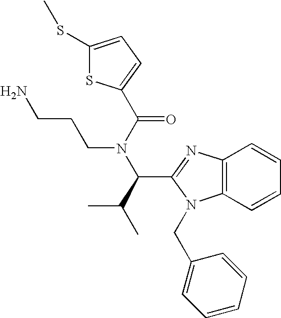

Example 30 was prepared analogously to Example 1. ¹H NMR (400 MHz, DMSO-D₆) δ ppm 0.63 (m, 1H) 0.74 (d, 3H) 0.93 (d, 3H) 1.23 (m, 3H) 1.31 (m, 1H) 2.21 (m, 2H) 2.83 (m, 1H) 2.94 (q, 2H) 3.47 (m, 1H) 3.68 (m, 1H) 5.53 (dd, 2H) 5.78 (d, 1H) 7.04 (m, 3H) 7.19-7.31 (band, 5H) 7.42-7.55 (band, 2H) 7.74 (m, 1H). ESI-MS: m/z 507.7 (M+H)⁺.

### Example 31

### (R)—N-(3-aminopropyl)-N-(1-(1-benzyl-1H-benzo[d]imidazol-2-yl)-2-methylpropyl)-4,5-dimethylthiophene-2-carboxamide

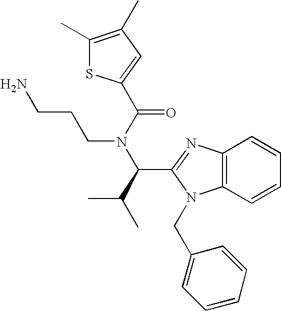

Example 31 was prepared analogously to Example 1. ¹H NMR (400 MHz, DMSO-D₆) δ ppm 0.70 (m, 4H) 0.92 (d, 3H) 1.35 (m, 1H) 2.06 (s, 3H) 2.23 (s, 2H) 2.30 (s, 3H) 2.81 (m, 1H) 3.49 (m, 1H) 3.69 (m, 1H) 5.53 (m, 2H) 5.77 (m, 1H) 7.07 (m, 2H) 7.19-7.31 (band, 6H) 7.50 (m, 1H) 7.72 (m, 1H). ESI-MS: m/z 475.2 (M+H)⁺.

### Example 32

### (R)—N-(3-aminopropyl)-N-(1-(1-benzyl-1H-benzo[d]imidazol-2-yl)-2-methylpropyl)-4-bromothiophene-2-carboxamide

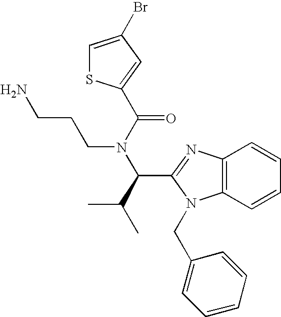

Example 32 was prepared analogously to Example 1. ¹H NMR (400 MHz, DMSO-D₆) δ ppm 0.72 (band, 4H) 0.93 (d, 3H) 1.23 (m, 1H) 2.20 (m, 2H) 2.80 (m, 1H) 3.47 (m, 1H) 3.68 (m, 1H) 5.53 (dd, 2H) 5.78 (d, 1H) 7.04 (d, 2H) 7.19-7.31 (band, 5H) 7.51 (m, 1H) 7.60 (s, 1H) 7.73 (m, 1H) 7.90 (s, 1H). m/z 525.2 (M+H)⁺.

### Example 33

### (R)—N-(3-aminopropyl)-N-(1-(1-benzyl-1H-benzo[d]imidazol-2-yl)-2-methylpropyl)-4,5-dibromothiophene-2-carboxamide

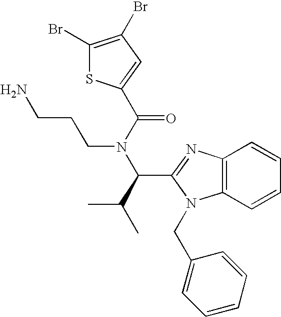

Example 33 was prepared analogously to Example 1. ¹H NMR (400 MHz, DMSO-D₆) δ ppm 0.72 (band, 4H) 0.93 (d, 3H) 1.40 (m, 1H) 2.32 (m, 2H) 2.79 (m, 1H) 3.49 (m, 1H) 3.70 (m, 1H) 5.51 (dd, 2H) 5.74 (d, 1H) 7.01 (m, 2H) 7.18-7.30 (band, 5H) 7.53 (m, 1H) 7.63 (s, 1H) 7.74 (m, 1H). ESI-MS: m/z 603.2 (M+H)⁺.

### Example 34

### (R)—N-(3-aminopropyl)-N-(1-(1-benzyl-1H-benzo[d]imidazol-2-yl)-2-methylpropyl)thieno[2,3-b]thiophene-2-carboxamide

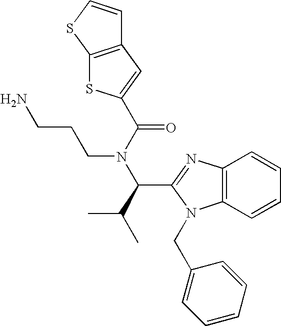

Example 34 was prepared analogously to Example 1. ¹H NMR (400 MHz, DMSO-D₆) δ ppm 0.70 (band, 4H) 0.95 (m, 3H) 1.45 (m, 1H) 2.30 (m, 2H) 2.83 (m, 1H) 3.57 (m, 1H) 3.78 (m, 1H) 5.54 (dd, 2H) 5.80 (m, 1H) 6.95-7.35 (band, 8H) 7.51 (m, 1H) 7.59-7.88 (band, 3H). ESI-MS: m/z 503.2 (M+H)⁺.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams) | mg (milligrams) |
| L (liters) | mL (milliliters) |
| μL (microliters) | psi (pounds per square inch) |
| M (molar) | mM (millimolar) |
| i.v. (intravenous) | Hz (Hertz) |
| MHz (megahertz) | mol (moles) |
| mmol (millimoles) | RT (ambient temperature) |
| min (minutes); h (hours) | mp (melting point) |
| TLC (thin layer chromatography) | Tr (retention time) |
| RP (reverse phase) | MeOH (methanol) |
| i-PrOH (isopropanol) | TEA (triethylamine) |
| TFA (trifluoroacetic acid) | TFAA (trifluoroacetic anhydride) |
| THF (tetrahydrofuran) | DMSO (dimethylsulfoxide) |
| EtOAc (ethyl acetate) | DME (1,2-dimethoxyethane) |
| DCM (dichloromethane) | DCE (dichloroethane) |
| DMF (N,N-dimethylformamide) | DMPU (N,N′-dimethylpropyleneurea) |
| CDI (1,1-carbonyldiimidazole) | IBCF (isobutyl chloroformate) |
| HOAc and AcOH (acetic acid) | HOSu (N-hydroxysuccinimide) |
| HOBT (1-hydroxybenzotriazole) | Et₂O (diethyl ether) |
| EDCI | BOC (tert-butyloxycarbonyl) |
| (ethylcarbodiimide hydrochloride) | |
| FMOC | DCC (dicyclohexylcarbodiimide) |
| (9-fluorenylmethoxycarbonyl) | |
| CBZ (benzyloxycarbonyl) | Ac (acetyl) |
| atm (atmosphere) | TMSE (2-(trimethylsilyl)ethyl) |
| TMS (trimethylsilyl) | TIPS (triisopropylsilyl) |
| TBS (t-butyldimethylsilyl) | DMAP (4-dimethylaminopyridine) |
| Me (methyl) | OMe (methoxy) |
| Et (ethyl) | |
| tBu (tert-butyl) | SPA (Scintillation Proximity Assay) |
| BOP (bis(2-oxo-3-oxazolidinyl)- | TBAF (tetra-n-butylammonium |
| phosphinic chloride) | fluoride) |
| mCPBA (meta-chloroperbenzoic | ATP (Adenosine Triphophatase) |
| acid | |
| BSA (Bovin Serum Albumin) | EDTA (Ethylenediaminetetraacetic |
| | acid) |
| NaOAc (sodium acetate) | MOPS (Morpholinepropanesulfonic |
| | acid) |
| HPLC (high pressure liquid | |
| chromatography) | |

All references to ether or Et₂O are to diethyl ether; brine refers to a saturated aqueous solution of NaCl. Unless otherwise indicated, all temperatures are expressed in ° C. (degrees Centigrade). All reactions conducted under an inert atmosphere at RT unless otherwise noted.

¹H NMR spectra were recorded on a Bruker Avance 400. Chemical shifts are expressed in parts per million (ppm). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) and compound purity data were acquired on a Waters ZQ LC/MS single quadrupole system equipped with electrospray ionization (ESI) source, UV detector (220 and 254 nm), and evaporative light scattering detector (ELSD). Thin-layer chromatography was performed on 0.25 mm E. Merck silica gel plates (60E-254), visualized with UV light, 5% ethanolic phosphomolybdic acid, Ninhydrin or p-anisaldehyde solution. Flash column chromatography was performed on silica gel (230-400 mesh, Merck).

It will be apparent to those skilled in the art that various modifications and variations can be made to the compounds, compositions, kits, and methods of the present invention without departing from the spirit or scope of the invention. Thus, it is intended that the present invention cover the modifications and variations of this invention provided they come within the scope of the appended claims and their equivalents.

1. A compound comprising the formula 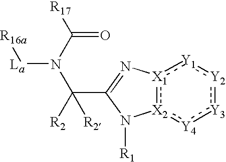
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR₆R₆′, NR₂₃, CO, S, SO, SO₂, and O, with the proviso that R₆′ and R₂₃ are each absent when the atom to which it is bound forms part of a double bond;
Y₂ is selected from the group consisting of CR₇R₇′, NR₂₄, CO, S, SO, SO₂, and O, with the proviso that R₇′ and R₂₄ are each absent when the atom to which it is bound forms part of a double bond;
Y₃ is selected from the group consisting of CR₈R₈′, NR₂₅, CO, S, SO, SO₂, and O, with the proviso that R₈′ and R₂₅ are each absent when the atom to which it is bound forms part of a double bond;
Y₄ is selected from the group consisting of CR₉R₉′, NR₂₆, CO, S, SO, SO₂, and O, with the proviso that R₉′ and R₂₆ are each absent when the atom to which it is bound forms part of a double bond;
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfunyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₆ₐ is an unsubstituted or substituted amino;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfonyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl, each substituted or unsubstituted; and
Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
with the provisos that Lₐ is not ethylene when R₁ₐ is dimethylamino and Lₐ is not hexyl when R₁₆ₐ is a 2-substituted pyridin-5-yl.

2. A compound comprising the formula 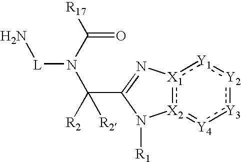
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR₆R₆′, NR₂₃, CO, S, SO, SO₂, and O, with the proviso that R₆′ and R₂₃ are each absent when the atom to which it is bound forms part of a double bond;
Y₂ is selected from the group consisting of CR₇R₇′, NR₂₄, CO, S, SO, SO₂, and O, with the proviso that R₇′ and R₂₄ are each absent when the atom to which it is bound forms part of a double bond;
Y₃ is selected from the group consisting of CR₈R₈′, NR₂₅, CO, S, SO, SO₂, and O, with the proviso that R₈′ and R₂₅ are each absent when the atom to which it is bound forms part of a double bond;
Y₄ is selected from the group consisting of CR₉R₉′, NR₂₆, CO, S, SO, SO₂, and O, with the proviso that R₉′ and R₂₆ are each absent when the atom to which it is bound forms part of a double bond;
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R² and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₄′ are taken together to form a 3- to 7-membered carbocyclic or heterocyclic ring, each substituted or unsubstituted;
R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

3. A compound comprising the formula 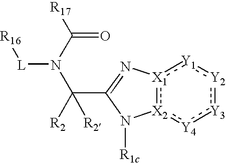
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR₆R₆′, NR₂₃, CO, S, SO, SO₂, and O, with the proviso that R₆′ and R₂₃ are each absent when the atom to which it is bound forms part of a double bond;
Y₂ is selected from the group consisting of CR₇R₇′, NR₂₄, CO, S, SO, SO₂, and O, with the proviso that R₇′ and R₂₄ are each absent when the atom to which it is bound forms part of a double bond;
Y₃ is selected from the group consisting of CR₈R₈′, NR₂₅, CO, S, SO, SO₂, and O, with the proviso that R₈′ and R₂₅ are each absent when the atom to which it is bound forms part of a double bond;
Y₄ is selected from the group consisting of CR₉R₉′, NR₂₆, CO, S, SO, SO₂, and O, with the proviso that R₉′ and R₂₆ are each absent when the atom to which it is bound forms part of a double bond;
R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkyl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl(C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl,
with the proviso that R₁₇ is not 3-chloro-benzo[b]thiophene-2-yl when R_{1c} is a 2-piperidinylmethyl-phenyl-methyl.

4. A compound comprising the formula 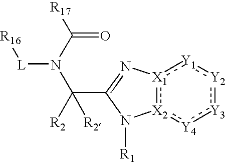
wherein:
X₁ and X₂ are each independently selected from the group consisting of CR_{1 8} and N, with the proviso that R₁₈ is absent when the carbon to which it is bound forms part of a double bond;
Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;
Y₂ is selected from the group consisting of CR_{7d}R_{7d}′, NR₂₄, CO, S, SO, SO₂, and O, with the proviso that R₇′ and R₂₄ are each absent when the atom to which it is bound forms part of a double bond;
Y₃ is selected from the group consisting of CR_{8d}R_{8d}′, NR₂₅, CO, S, SO, SO₂, and O, with the proviso that R₈′ and R₂₅ are each absent when the atom to which it is bound forms part of a double bond;
Y₄ is selected from the group consisting of CR_{9d}R_{9d}′, NR₂₆, CO, S, SO, SO₂, and O, with the proviso that R₉′ and R₂₆ are each absent when the atom to which it is bound forms part of a double bond;
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R_{6d}, R_{6d}′, R_{7d}, R_{7d}′, R_{8d}, R_{8d}′, R_{9d} and R_{9d}′ are each independently hydrogen or are each selected from the group consisting of halo, halo(C₁₋₆)alkyl, amino, nitro, cyano, thio, (C₁₋₃)alkyl, cycloalkyl, hetero(C₃₋₁₂)cycloalkyl, carbonyl(C₁₋₅)alkyl, thiocarbonyl(C₁₋₅)alkyl, sulfonyl (C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;
R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

5. A compound comprising the formula 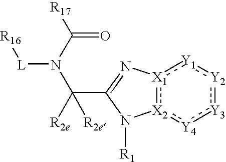
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR₆R₆′, NR₂₃, CO, S, SO, SO₂, and O, with the proviso that R₆′ and R₂₃ are each absent when the atom to which it is bound forms part of a double bond;
Y₂ is selected from the group consisting of CR₇R₇′, NR₂₄, CO, S, SO, SO₂, and O, with the proviso that R₇′ and R₂₄ are each absent when the atom to which it is bound forms part of a double bond;
Y₃ is selected from the group consisting of CR₈R₈′, NR₂₅, CO, S, SO, SO₂, and O, with the proviso that R₈′ and R₂₅ are each absent when the atom to which it is bound forms part of a double bond;
Y₄ is selected from the group consisting of CR₉R₉′, NR₂₆, CO, S, SO, SO₂, and O, with the proviso that R₉′ and R₂₆ are each absent when the atom to which it is bound forms part of a double bond;
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;
R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂′ are taken together to form a 3- to 7-membered ring, each substituted or unsubstituted;
R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

6. The compound according to claim 1, comprising the formula 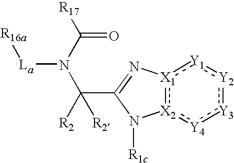
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR₆R₆′, NR₂₃, CO, S, SO, SO₂, and O, with the proviso that R₆′ and R₂₃ are each absent when the atom to which it is bound forms part of a double bond;
Y₂ is selected from the group consisting of CR₇R₇′, NR₂₄, CO, S, SO, SO₂, and O, with the proviso that R₇′ and R₂₄ are each absent when the atom to which it is bound forms part of a double bond;
Y₃ is selected from the group consisting of CR₈R₈′, NR₂₅, CO, S, SO, SO₂, and O, with the proviso that R₈′ and R₂₅ are each absent when the atom to which it is bound forms part of a double bond;
Y₄ is selected from the group consisting of CR₉R₉′, NR₂₆, CO, S, SO, SO₂, and O, with the proviso that R₉′ and R₂₆ are each absent when the atom to which it is bound forms part of a double bond;
R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₆ₐ is an unsubstituted or substituted amino;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)_{alkyl,} carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

7. The compound according to claim 1, comprising the formula 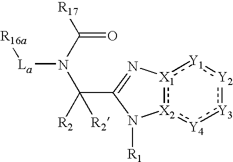
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;
Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;
Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;
Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl(C₁₋₅)alkyl, sulfonyl (C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;
R₁₆ₐ is an unsubstituted or substituted amino;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

8. The compound according to claim 1, comprising the formula 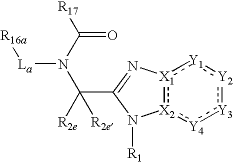
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR₆R₆′, NR₂₃, CO, S, SO, SO₂, and O, with the proviso that R₆′ and R₂₃ are each absent when the atom to which it is bound forms part of a double bond;
Y₂ is selected from the group consisting of CR₇R₇′, NR₂₄, CO, S, SO, SO₂, and O, with the proviso that R₇′ and R₂₄ are each absent when the atom to which it is bound forms part of a double bond;
Y₃ is selected from the group consisting of CR₈R₈′, NR₂₅, CO, S, SO, SO₂, and O, with the proviso that R₈′ and R₂₅ are each absent when the atom to which it is bound forms part of a double bond;
Y₄ is selected from the group consisting of CR₉R₉′, NR₂₆, CO, S, SO, SO₂, and O, with the proviso that R₉′ and R₂₆ are each absent when the atom to which it is bound forms part of a double bond;
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;
R₂′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂′ are taken together to form a 3- to 7-membered ring, each substituted or unsubstituted;
R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₆ₐ is an unsubstituted or substituted amino;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

9. The compound according to claim 1, comprising the formula 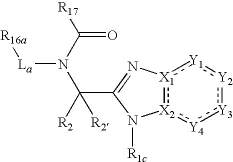
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;
Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;
Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;
Y₄ is selected from the group consisting of CR_{9d}R_{9d}′, NR₂₆, CO, S, SO, SO₂, and O, with the proviso that R₉′ and R₂₆ are each absent when the atom to which it is bound forms part of a double bond;
R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl(C₁₋₅)alkyl, sulfonyl (C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;
R₁₆ₐ is an unsubstituted or substituted amino;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

10. The compound according to claim 1, comprising the formula 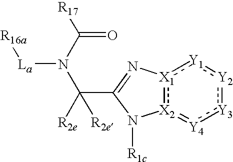
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR₆R₆′, NR₂₃, CO, S, SO, SO₂, and O, with the proviso that R₆′ and R₂₃ are each absent when the atom to which it is bound forms part of a double bond;
Y₂ is selected from the group consisting of CR₇R₇′, NR₂₄, CO, S, SO, SO₂, and O, with the proviso that R₇′ and R₂₄ are each absent when the atom to which it is bound forms part of a double bond;
Y₃ is selected from the group consisting of CR₈R₈′, NR₂₅, CO, S, SO, SO₂, and O, with the proviso that R₈′ and R₂₅ are each absent when the atom to which it is bound forms part of a double bond;
Y₄ is selected from the group consisting of CR₉R₉′, NR₂₆, CO, S, SO, SO₂, and O, with the proviso that R₉′ and R₂₅ are each absent when the atom to which it is bound forms part of a double bond;
R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;
R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;
R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₆ₐ is an unsubstituted or substituted amino;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, hetero aryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₅ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

11. The compound according to claim 1, comprising the formula 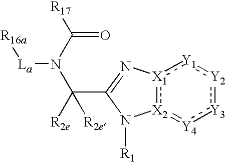
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;
Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;
Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;
Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl hetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;
R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;
R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl(C₁₋₅)alkyl, sulfonyl (C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;
R₁₆ₐ is an unsubstituted or substituted amino;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

12. The compound according to claim 2, comprising the formula 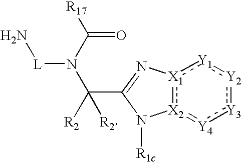
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR₆R₆′, NR₂₃, CO, S, SO, SO₂, and O, with the proviso that R₆′ and R₂₃ are each absent when the atom to which it is bound forms part of a double bond;
Y₂ is selected from the group consisting of CR₇R₇′, NR₂₄, CO, S, SO, SO₂, and O, with the proviso that R₇′ and R₂₄ are each absent when the atom to which it is bound forms part of a double bond;
Y₃ is selected from the group consisting of CR₈R₈′, NR₂₅, CO, S, SO, SO₂, and O, with the proviso that R₈′ and R₂₅ are each absent when the atom to which it is bound forms part of a double bond;
Y₄ is selected from the group consisting of CR₉R₉′, NR₂₆, CO, S, SO, SO₂, and O, with the proviso that R₉′ and R₂₆ are each absent when the atom to which it is bound forms part of a double bond;
R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl sulfonyl(C₁₋₅)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

13. The compound according to claim 2, comprising the formula 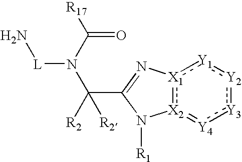
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;
Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;
Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;
Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁2)cycloalkyl, carbonyl(C₁₋₅)alkyl, thiocarbonyl(C₁₋₅)alkyl, sulfonyl (C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₄₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
L is a bond, or a linker comprising a backbone chain of 1 to 10 atoms comprising C, N, O, or S and may be optionally substituted with halo, halo(C₁₋₁₀)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

14. The compound according to claim 2, comprising the formula 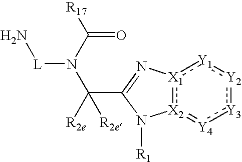
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR₆R₆′, NR₂₃, CO, S, SO, SO₂, and O, with the proviso that R₆′ and R₂₃ are each absent when the atom to which it is bound forms part of a double bond;
Y₂ is selected from the group consisting of CR₇R₇′, NR₂₄, CO, S, SO, SO₂, and O, with the proviso that R₇′ and R₂₄ are each absent when the atom to which it is bound forms part of a double bond;
Y₃ is selected from the group consisting of CR₈R₈′, NR₂₅, CO, S, SO, SO₂, and O, with the proviso that R₈′ and R₂₅ are each absent when the atom to which it is bound forms part of a double bond;
Y₄ is selected from the group consisting of CR₉R₉′, NR₂₆, CO, S, SO, SO₂, and O, with the proviso that R₉′ and R₂₆ are each absent when the atom to which it is bound forms part of a double bond;
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;
R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;
R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₆)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

15. The compound according to claim 2, comprising the formula 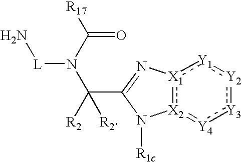
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR_{6d}R_{6d}′, NR₂₃, CO, S, SO, SO₂, and O, with the proviso that R_{5d}′ and R₂₃ are each absent when the atom to which it is bound forms part of a double bond;
Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;
Y₃ is selected from the group consisting of CR_{8d}R_{8d}′, NR₂₅, CO, S, SO, SO₂, and O, with the proviso that R₈′ and R₂₅ are each absent when the atom to which it is bound forms part of a double bond;
Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;
R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R_{6d}, R_{6d}′, R_{7d}, R_{7d}′, R_{8d}, R₈′, R_{9d}, and R_{9d}′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

16. The compound according to claim 2, comprising the formula 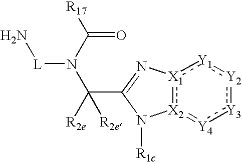
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR₆R₆′, NR₂₃, CO, S, SO, SO₂, and O, with the proviso that R₆′ and R₂₃ are each absent when the atom to which it is bound forms part of a double bond;
Y₂ is selected from the group consisting of CR₇R₇′, NR₂₄, CO, S, SO, SO₂, and O, with the proviso that R₇′ and R₂₄ are each absent when the atom to which it is bound forms part of a double bond;
Y₃ is selected from the group consisting of CR₈R₈′, NR₂₅, CO, S, SO, SO₂, and O, with the proviso that R₈′ and R₂₅ are each absent when the atom to which it is bound forms part of a double bond;
Y₄ is selected from the group consisting of CR₉R₉′, NR₂₆, CO, S, SO, SO₂, and O, with the proviso that R₉′ and R₂₆ are each absent when the atom to which it is bound forms part of a double bond;
R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;
R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂′ are taken together to form a 3- to 7-membered ring, each substituted or unsubstituted;
R₆, R₆′, R₇, R₇′, R₈, R₈′, R₉, and R₉′ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

17. The compound according to claim 2, comprising the formula 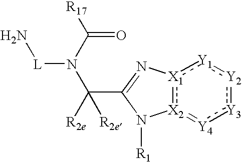
wherein:
X₁ and X₂ are each independently selected from the group consisting of CR₁₈ and N, with the proviso that R₁₈ is absent when the carbon to which it is bound forms part of a double bond;
Y₁ is selected from the group consisting of _{CR}_{6d}R_{6d}′, NR₂₃, CO, S, SO, SO₂, and O, with the proviso that R_{6d}′ and R₂₃ are each absent when the atom to which it is bound forms part of a double bond;
Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;
Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;
Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, hetero aryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;
R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂′ are taken together to form a 3- to 7-membered ring, each substituted or unsubstituted;
R_{6d}, R_{6d}′, R_{7d}, R_{7d}′, R_{8d}, R₈′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl(C₁₋₅)alkyl, sulfonyl(C₁₋₃)alkyl, sulfinyl(C_{f-3})alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

18. The compound according to claim 1, comprising the formula 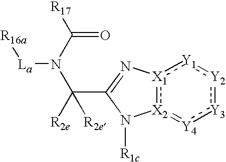
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;
Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;
Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;
Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;
R_{1c} is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;
R₂ₑ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;
R_{6d}, R₆′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;
R₁₆ₐ is an unsubstituted or substituted amino;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted.

19. The compound according to claim 2, comprising the formula 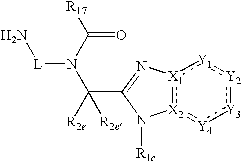
wherein:
X₁ and X₂ are each independently selected from the group consisting of CR₁₈ and N, with the proviso that R₁₈ is absent when the carbon to which it is bound forms part of a double bond;
Y₁ is selected from the group consisting of CR_{6d}R_{6d}′, NR₂₃, CO, S, SO, SO₂, and O, with the proviso that R_{6d}′ and R₂₃ are each absent when the atom to which it is bound forms part of a double bond;
Y₂ is selected from the group consisting of CR_{7d}R_{7d}′, NR₂₄, CO, S, SO, SO₂, and O, with the proviso that R_{7d}′ and R₂₄ are each absent when the atom to which it is bound forms part of a double bond;
Y₃ is selected from the group consisting of CR_{8d}R_{8d}′, NR₂₅, CO, S, SO, SO₂, and O, with the proviso that R_{8d}′ and R₂₅ are each absent when the atom to which it is bound forms part of a double bond;
Y₄ is selected from the group consisting of CR_{9d}R_{9d}′, NR₂₆, CO, S, SO, SO₂, and O, with the proviso that R_{9d}′ and R₂₆ are each absent when the atom to which it is bound forms part of a double bond;
R₁ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;
R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂′ are taken together to form a 3- to 7-membered ring, each substituted or unsubstituted;
R_{6d}, R_{6d}′, R_{7d}, R_{7d}′, R_{8d}, R_{8d}′, R_{9d}, and R_{9d}′ are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R₈_{d}, and R_{9d} is not H;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
R₁₈ is selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, halo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted;
R₂₃, R₂₄, R₂₅, and R₂₆ are each independently selected from the group consisting of hydrogen, carbonyl, sulfonamido, imino, sulfonyl, sulfinyl, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(_{C}₁₋₁₀)alkyl, imino(C₁₋₃)alkyl, (C₁₋₆)alkylaryl, aryl(C₁₋₆)alkyl, heteroaryl, (C₁₋₆)alkylheteroaryl, and heteroaryl(C₁₋₆)alkyl each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

20. The compound according to claim 1, comprising the formula 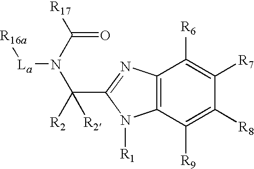
wherein:
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R₆, R₇, R₈, and R₉ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₆ₐ is an unsubstituted or substituted amino;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylhetero aryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, (C₁₋₆)heteroalkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
with the provisos that Lₐ is not ethyl when R₁₆ₐ is dimethylamino and Lₐ is not hexyl when R₁₆ₐ is a 2-substituted pyridine-5-yl.

21. The compound according to claim 2, comprising the formula 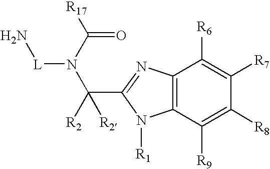
wherein:
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R₆, R₇, R₈ and R₉ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted;
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

22. The compound according to claim 3, comprising the formula 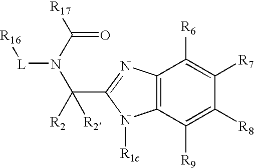
wherein:
R₁ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R₆, R₇, R₈, and R₉ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.
with the proviso that R₁₇ is not 3-chloro-benzo[b]thiophene-2-yl when R₁ is a 2-piperidinylmethyl-phenyl-methyl.

23. The compound according to claim 4, comprising the formula 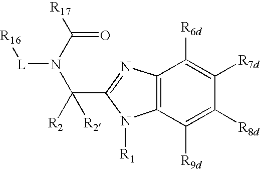
wherein:
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R_{6d}, R_{7d}, R_{8d} and R_{9d} are each independently hydrogen or are each selected from the group consisting of halo, halo(C₁₋₆)alkyl, amino, nitro, cyano, thio, (C₁₋₆)alkyl, cycloalkyl, hetero(C₃₋₁₂)cycloalkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, and a substituted or unsubstituted 4, 5, 6 or 7 membered ring, with the proviso that at least one of R_{6d}, R_{7d}, R_{8d}, and R_{9d} is not H;
R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

24. The compound according to claim 5, comprising the formula 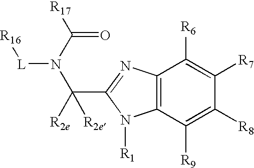
wherein:
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ₑ is selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid;
R₂ₑ′ is selected from the group consisting of hydrogen, (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ₑ and R₂ₑ′ are taken together to form a 3- to 7-membered ring, each substituted or unsubstituted;
R₆, R₇, R₈, and R₉ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl sulfonyl(C₁₋₅)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring;
R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₁₂)cycloalkyl, (C₁₋₆)alkylhetero(C₃₋₇)cycloalkyl, hetero(C₃₋₇)cycloalkyl(C₁₋₆)alkyl, (C₉₋₁₂)bicycloalkyl, (C₁₋₆)alkyl(C₉₋₁₂)bicycloalkyl, (C₉₋₁₂)bicycloalkyl(C₁₋₆)alkyl, hetero(C₃₋₁₂)bicycloalkyl, (C₁₋₆)alkylhetero(C₃₋₁₂)bicycloalkyl, hetero(C₃₋₁₂)bicycloalkyl(C₁₋₆)alkyl, aryl, (C₁₋₆)alkylaryl, aryl(C₁₋₆)alkyl, heteroaryl, (C₁₋₆)alkylhetero aryl, heteroaryl(C₁₋₆)alkyl, halo(C₁₋₁₀)alkyl, (C₃₋₁₂)cycloalkyl(C₁₋₁₀)alkyl, and amino(C₁₋₁₀)alkyl, each substituted or unsubstituted; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

25. The compound according to claim 1, wherein X₁ and X₂ are both C and form part of a double bond.

26. The compound according to claim 1, wherein Y₁ is CR₆ and forms part of a double bond.

27. The compound according to claim 1, wherein Y₂ is CR₇ and forms part of a double bond.

28. The compound according to claim 1, wherein Y₃ is CR₈ and forms part of a double bond.

29. The compound according to claim 1, wherein Y₄ is CR₉ and forms part of a double bond.

30. The compound according to claim 1, wherein R₁ is selected from the group consisting of (C₁₋₆)alkyl, aryl(C₁₋₆)alkyl, and heteroaryl(C₁₋₆)alkyl, each substituted or unsubstituted.

31. The compound according to claim 1, wherein R₁ is selected from the group consisting of methyl, benzyl, thiophene-yl-methyl, and pyridinylmethyl, each substituted or unsubstituted.

32. The compound according to claim 3, wherein R_{1c} is selected from the group consisting of (C₁₋₆)alkyl, (C₁₋₆)alkyloxy(C₁₋₆)alkyl, aryl, (C₁₋₆)alkylaryl, heteroaryl, (C₁₋₆)alkylheteroaryl, and hetero(C₃₋₇)cycloalkyl, each substituted or unsubstituted.

33. The compound according to claim 1, wherein R₁ is selected from the group consisting of (C₁₋₆)alkyl, aryl, (C₁₋₆)alkylaryl, heteroaryl, (C₁₋₆)alkylheteroaryl, and hetero(C₃₋₇)cycloalkyl, each substituted or unsubstituted.

34. The compound according to claim 1, wherein R₁ is selected from the group consisting of benzyl, phenyl, methylthiophene-yl, and methylfuranyl, each substituted or unsubstituted.

35. The compound according to claim 1, wherein R₁ is selected from the group consisting of methyl, ethyl, propyl, methoxymethyl, methoxyethyl, ethoxymethyl, ethoxyethyl, phenyl, 4-morpholinyl-(C₂₋₆)alkenyl, 4-morpholin-4-yl-buten-2-yl, pyrrolidin-1-yl-(C₂₋₆)alkenyl, 2-, 3-, or 4-halophenyl, 2-, 3- or 4-(C₁₋₃)alkylphenyl, 2,4-di-(C₁₋₃)alkylphenyl, 3,4-di-(C₁₋₃)alkylphenyl, 3-, or 4-(C₁₋₃)alkoxyphenyl, 2-(C₁₋₃)alkyl-3-halophenyl, 2,4-di-halophenyl, 3,4-di-halophenyl, 2-(C₁₋₃)alkyl-4-halophenyl, 2-, 3- or 4-(C₁₋₃)alkylbenzyl, 2,4-di-(C₁₋₃)alkylbenzyl, 3,4-di-(C₁₋₃)alkylbenzyl, 3 or 4-(C₁₋₃)alkoxybenzyl, 2-, 3- or 4-halobenzyl, 2-(C₁₋₃)alkyl-3-halobenzyl, 2,4-di-halobenzyl, 3,4-di-halobenzyl, 2-(C₁₋₃)alkyl-4-halobenzyl, and (C₁₋₅)alkoxycarbonyl-(C₁₋₆)alkyl, each optionally further substituted.

36. The compound according to claim 1, wherein R₁ is selected from the group consisting of naphthyl, naphthalen(C₁₋₃)alkyl, 2-tetrahydrofuran-methyl-, piperidinyl, piperidino(C₁₋₃)alkyl, 2-imidazol-1-ylmethyl-benzyl, 4-morpholinyl, 4-morpholino(C₁₋₃)alkyl, 4-piperazinyl, 2-piperidin-1-ylmethyl-benzyl, 2-(3,3-dimetyl-piperidin-1-ylmethyl)-benzyl, 2-diethylaminomethyl-benzyl, 2-((ethyl-methyl-amino)methyl)-benzyl, 2-(4-methyl-piperidin-1-ylmethyl)-benzyl, (2-morpholin-4-yl-methylbenzyl), 2-(4-methyl-piperazin-1-ylmethyl)-benzyl, 4-piperazino(C₁₋₃)alkyl, pyrrolidinyl, pyrrolidino(C₁₋₃)alkyl, perhydropyrrolizinyl, perhydropyrrolizino(C₁₋₃)alkyl, 1,4-diazaperhydroepinyl, 1,4-diazaperhydroepinyl(C₁₋₃)alkyl, 1,3-dioxanyl, 1,4-dioxanyl(C₁₋₃)alkyl, 1,3-dioxanyl, and 1,4-dioxanyl(C₁₋₃)alkyl, each substituted or unsubstituted.

37. The compound according to claim 1, wherein R₂ is selected from the group consisting of hydrogen and (C₁₋₆)alkyl, each substituted or unsubstituted.

38. The compound according to claim 1, wherein R₂ is selected from the group consisting of methyl, ethyl and propyl, each substituted or unsubstituted.

39. The compound according to claim 1, wherein R₂′ is hydrogen.

40. The compound according to claim 1, wherein R₂ and R₂′ are each independently hydrogen or are each independently selected from the group consisting of (C₁₋₆)alkyl, (C₁₋₃)alkyloxy(C₁₋₃)alkyl, aryl, (C₁₋₁₀)alkylaryl, heteroaryl, (C₃₋₁₂)cycloalkyl, hetero(C₃₋₁₂)cycloalkyl, (C₁₋₆)alkylheteroaryl, and the side chain of an amino acid, or R₂ and R₂′ are taken together to form a 3- to 7-membered ring, each substituted or unsubstituted.

41. The compound according to claim 1, wherein R₂ and R₂′ are each independently hydrogen or the side chain of an amino acid.

42. The compound according to claim 1, wherein R₂ and R₂′ are taken together to form a 3- to 7-membered ring, each substituted or unsubstituted.

43. The compound according to claim 1, wherein R₂ and R₂′ are each independently hydrogen or are each selected from the group consisting of an unsubstituted or substituted (R) or (S) (C₁₋₅)alkyl.

44. The compound according to claim 1, wherein R₂ and R₂′ are each independently hydrogen or are each selected from the group consisting of an unsubstituted or substituted (R) (C₁₋₅)alkyl.

45. The compound according to claim 1, wherein the stereogenic center to which R₂ and R₂′ are attached is in the R configuration.

46. The compound according to claim 5, wherein R₂ₑ is selected from the group consisting of (C₁₋₁₀)alkyl, (C₁₋₆)alkyloxy(C₁₋₆)alkyl, aryl(C₁₋₁₀)alkyl, aryl, (C₁₋₁₀)alkylaryl, heteroaryl, heteroaryl(C₁₋₆)alkyl, hetero(C₃₋₁₂)cycloalkyl, (C₁₋₆)alkylheteroaryl, and the side chain of an amino acid.

47. The compound according to claim 5, wherein R₂ₑ′ is selected from the group consisting of hydrogen, (C₁₋₁₀)alkyl, (C₁₋₆)alkyloxy(C₁₋₆)alkyl, aryl(C₁₋₁₀)alkyl, aryl, (C₁₋₁₀)alkylaryl, heteroaryl, heteroaryl(C₁₋₆)alkyl, hetero(C₃₋₁₂)cycloalkyl, (C₁₋₆)alkylheteroaryl, and the side chain of an amino acid, or R₂ₑ and R₂₀′ are taken together to form a 3- to 7-membered ring, each substituted or unsubstituted.

48. The compound according to claim 1, wherein R₁₇ is selected from the group consisting of aryl(C₁₋₆)alkyl, (C₃₋₇)cycloalkyl, hetero(C₃₋₇)cycloalkyl, haloaryl, (C₁₋₆)alkylaryl, (C₁₋₆)alkoxyaryl, cyanoaryl, heteroaryl, haloheteroaryl, (C₁₋₆)alkylheteroaryl, (C₁₋₆)alkyl-thio-heteroaryl, and heterobicycloaryl, each substituted or unsubstituted.

49. The compound according to claim 1, wherein R₁₇ is selected from the group consisting of hydrogen, thio, hydroxy, alkoxy, aryloxy, heteroaryloxy, amino, (C₁₋₁₀)alkylamino, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, halo(C₁₋₁₀)alkyl, (C₃₋₁₂)cycloalkyl(C₁₋₁₀)alkyl, amino(C₁₋₁₀)alkyl, aryl, heteroaryl, (C₉₋₁₂)bicycloaryl, and hetero(C₄₋₁₂)bicycloaryl, each substituted or unsubstituted.

50. The compound according to claim 1, wherein R₁₈ is selected from the group consisting of hydrogen, nitro, cyano, thio, hydroxy, alkoxy, carbonyl, amino, (C₁₋₁₀)alkylamino, sulfonamido, imino, sulfonyl, sulfinyl, (C₁₋₁₀)alkyl, (C₃₋₇)cycloalkyl, hetero(C₃₋₇)cycloalkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted.

51. The compound according to claim 1, wherein R₁₈ is selected from the group consisting of hydrogen, halo, and (C₁₋₆)alkyl, each substituted or unsubstituted.

52. The compound according to claim 1, wherein R₂₃, R₂₄, R₂₅, and R₂₆ are each independently selected from the group consisting of hydrogen, (C₁₋₁₀)alkylamino, (C₁₋₅)alkyl, (C₃₋₇)cycloalkyl, hetero(C₃₋₇)cycloalkyl, halo(C₁₋₁₀)alkyl, aryl, and heteroaryl, each substituted or unsubstituted.

53. The compound according to claim 1, wherein R₁₇ is hydrogen or is selected from the group consisting of halo(C₁₋₆)alkyl, amino, (C₁₋₁₅)alkyl, cycloalkyl, hetero(C₃₋₁₂)cycloalkyl, aryl, heteroaryl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, aryloxy, heteroaryloxy, each substituted or unsubstituted, and a substituted or unsubstituted 4, 5, 6 or 7 membered ring.

54. The compound according to claim 1, wherein R₁₇ is hydrogen or is selected from the group consisting of (C₁₋₁₅)alkyl, aryl, aryl(C₁₋₃)alkyl, heteroaryl, heteroaryl(C₁₋₃)alkyl, each substituted or unsubstituted, R₁₄O(C₁₋₆)alkyl where R₁₄ is H or is selected from the group consisting of (C₁₋₃)alkyl, aryl, aryl(C₁₋₃)alkyl, heteroaryl, heteroaryl(C₁₋₃)alkyl, each substituted or unsubstituted, (C₁₋₃)alkoxyCO(C₁₋₃)alkyl, [(C₁₋₃)alkoxyl(C₁₋₃)alkyl]₁₋₃, aryl[(C₁₋₃)alkoxy(C₁₋₃)alkyl]₁₋₃, [(C₁₋₃)alkoxy(C₁₋₃)alkyl]₁₋₃aryl, and [(C₁₋₃)alkoxy(C₁₋₃)alkyl]₁₋₃aryl, each substituted or unsubstituted, or R₁₄R₁₅N(C₁₋₆)alkyl where R₁₄ and R₁₅ are each independently H or (C₁₋₃)alkyl, aryl, aryl(C₁₋₃)alkyl, heteroaryl, and heteroaryl(C₁₋₃)alkyl, each substituted or unsubstituted.

55. The compound according to claim 1, wherein R₁₇ is selected from the group consisting of styrenyl, naphthyl, 2-, 3- or 4-(C₁₋₅)alkylbenzyl, and 2-, 3- or 4-(C₁₋₅)alkoxybenzyl, each substituted or unsubstituted.

56. The compound according to claim 1, wherein R₁₇ is selected from the group consisting of phenyl, methylenedioxyphenyl, biphenyl, 2-, 3- or 4-(C₁₋₅)alkylphenyl, 2-, 3-, or 4-halophenyl, 2- or 4-CN-phenyl, 3- or 4-NO₂-phenyl, 2-, 3-, or 4-MeO-phenyl, 2-, 3-, or 4-CF₃-phenyl, 2-, 3-, or 4-CF₃O-phenyl, 2- or 4(C₁₋₅)alkylSO₂phenyl, 2- or 4-(C₁₋₅)alkylSOphenyl, 2- or 4-[(C₁₋₄)alkylOCO]phenyl, 2,3-, 2,4-, 2,5- or 2,6-dihalophenyl, 3,4-dihalophenyl, 3,5-dihalophenyl, 2-, 3- or 4-carboxyphenyl, 2,3-, 2,4-, 2,5- or 2,6-, or 3,5-di(C₁₋₄)alkylphenyl, 3,5-di(C₁₋₄)alkoxyphenyl, 3,5-di(CF₃)-phenyl, perfluoro(C₁₋₄)alkylphenyl, and F₅-phenyl, each further substituted or unsubstituted.

57. The compound according to claim 1, wherein R₁₇ is selected from the group consisting of (C₁₋₅)alkylHNCH₂—, arylNHCH₂—, alkylaryl HNCH₂—, heteroarylHNCH₂—, and alkylheteroarylHNCH₂—, each substituted or unsubstituted.

58. The compound according to claim 1, wherein R₁₇ is selected from the group consisting of (C₁₋₅)alkylNH—, ((C₁₋₅)alkyl)₂N, ((C₁₋₅)alkyl)₂NCH₂—, phenyl-NH—, phenyl-N(C₁₋₅)alkyl -, 2-, 3-, or 4-halophenyl-NH—, and 2-, 3-, or 4-(C₁₋₅)alkylphenyl-NH—, each substituted or unsubstituted.

59. The compound according to claim 1, wherein R₁₇ is selected from the group consisting of styrenyl-CH₂—, and naphthyl-CH₂—, each substituted or unsubstituted.

60. The compound according to claim 1, wherein R₁₇ is selected from the group consisting of benzyl, methylenedioxybenzyl, biphenyl-CH₂—, 2-, 3- or 4-(C₁₋₅)alkylbenzyl, 2-, 3-, or 4-halobenzyl, 2- or 4-CN-benzyl, 3- or 4-NO₂-benzyl, 2-, 3- or 4-MeO-benzyl, 2-, 3-, or 4-CF₃-benzyl, 2-, 3-, or 4-CF₃O-benzyl, 2- or 4-(C₁₋₅)alkylSO₂benzyl, 2- or 4-[(C₁₋₄alkylOCO]benzyl, 2,3-, 2,4-, 2,5- or 2,6-dihalobenzyl, 3,4-dihalobenzyl, 3,5-dihalobenzyl, 2-, 3- or 4-carboxybenzyl, 2,3-, 2,4-, 2,5- or 2,6-, or 3,5-di(C₁₋₄)alkylbenzyl, 3,5-di(C₁₋₄)alkoxybenzyl, 3,5-di(CF₃)-benzyl, perfluoro(C₁₋₄)alkylbenzyl, and F₅-benzyl, each further substituted or unsubstituted.

61. The compound according to claim 1, wherein R₁₇ is selected from the group consisting of (C₁₋₅)alkylNH—CH₂—, ((C₁₋₅)alkyl)₂NCH₂—, phenyl-NH—CH₂—, phenyl-N(C₁₋₅)alkyl-, 2-, 3-, or 4-halophenyl-NH—CH₂—, and 2-, 3-, or 4-(C₁₋₅)alkylphenyl-NH—CH₂—, each substituted or unsubstituted.

62. The compound according to claim 1, wherein R₁₇ is selected from the group consisting of 2-, 3- or 4-pyridyl, 2-(C₁₄)alkyl-pyrid-5-yl, imidazolyl, pyrazolyl, quinolinyl, quinoxalinyl, quinazolinyl, pyrrolyl, pyrazolyl, benzofuranyl, benzothiophenyl, benzimidazolyl, indolyl, furanyl, and tetrahydrofuranyl, each substituted or unsubstituted.

63. The compound according to claim 1, wherein R₁₇ is selected from the group consisting of halo(C₁₋₆)alkyl, cycloalkyl, heterocycloalkyl, aryl, heteroaryl, carbonyl(C₁₋₅)alkyl, thiocarbonyl(C₁₋₅)alkyl, sulfonyl(C₁₋₃)alkyl, sulfinyl(C₁₋₃)alkyl, and imino(C₁₋₃)alkyl, each substituted or unsubstituted.

64. The compound according to claim 1, wherein R₁₇ is selected from the group consisting of (C₁₋₁₀)alkyl, halo(C₁₋₆)alkyl, aryl(C₁₋₁₀)alkyl, cycloalkyl, aryl, heteroaryl, (C₁₋₁₀)alkylaryl, heteroaryl, heteroaryl(C₁₋₆)alkyl, hetero(C₃₋₁₂)cycloalkyl, and (C₁₋₁₀)alkylhetero(C₃₋₈)aryl, each substituted or unsubstituted, and wherein R₁₁ is hydrogen or is selected from the group consisting of (C₁₋₁₀)alkyl, halo(C₁₋₆)alkyl, aryl(C₁₋₁₀)alkyl, cycloalkyl, aryl, heteroaryl, (C₁₋₁₀)alkylaryl, heteroaryl, heterocycloalkyl, heteroaryl(C₁₋₆)alkyl, hetero(C₃₋₁₂)cycloalkyl, and (C₁₋₁₀)alkylhetero(C₃₋₈)aryl, each substituted or unsubstituted.

65. The compound according to claim 1, wherein R₁₇ is selected from the group consisting of (C₁₋₁₀)alkyl, halo(C₁₋₆)alkyl, aryl(C₁₋₁₀)alkyl, cycloalkyl, aryl, heteroaryl, (C₁₋₁₀)alkylaryl, heteroaryl, hetero(C₃₋₁₂)cycloalkyl, heteroaryl(C₁₋₆)alkyl, hetero(C₃₋₁₂)cycloalkyl, and (C₁₋₁₀)alkylhetero(C₃₋₈)aryl, each substituted or unsubstituted.

66. The compound according to claim 1, wherein R₁₇ is selected from the group consisting of (C₁₋₁₀)alkyl, aryl(C₁₋₁₀)alkyl, aryl, heteroaryl, heteroaryl(C₁₋₆)alkyl, and hetero(C₃₋₁₂)cycloalkyl, each substituted or unsubstituted.

67. The compound according to claim 1, wherein R₁₇ is selected from the group consisting of (C₁₋₃)alkyl, phenyl(C₁₋₃)alkyl, phenyl, biphenyl, naphthyl, naphthyl(C₁₋₃)alkyl, heteroaryl, and heteroaryl(C₁₋₆)alkyl, each substituted or unsubstituted.

68. The compound according to claim 1, wherein R₁₇ is selected from the group consisting of 3- or 4-halophenyl, 2-(C₁₋₃)alkyl-5-halophenyl, 2-, 3- or 4-halophenyl, 2-, 3- or 4-halobenzyl, 2,3-, 2,4-, 2,5-, or 3,4-dihalophenyl, 4-cyanophenyl, 4-(pyrrolidiny-1-yl)phenyl, 4-(C₁₋₃)alkylSO₂phenyl, 4-(C₁₋₃)alkylSOphenyl, 4-C₁₋₆)alkoxyphenyl, 2-, 3- or 4-(C₁₋₃)alkylthiophenyl, 4-CF₃-thiophenyl, 4-(C₁₋₃)alkylNH—(C₁₋₃)alkylphenyl, 4-morpholin-4-yl-(C₁₋₃)alkylphenyl, and 4-(piperidin-1-yl-(C₁₋₃)alkyl)phenyl, each unsubstituted or further substituted.

69. The compound according to claim 1, wherein R₁₇ is selected from the group consisting of 2-, 3- or 4-(C₁₋₃)alkylphenyl, 2-, 3- or 4)C₁₋₃)alkylbenzyl, 8-halo-naphthalen-1-yl, napthalen-1-yl-CH′CH—, 2-(C₁₋₃)alkylphenyl-CH═CH—, 2,3-dihalophenyl-CH═CH—, 2,5-dihalophenyl, 2,6-dihalophenyl-CH═CH—, 2-halo-4,5-dimethoxyphenyl, 2-halo-3,4-dimethoxyphenyl, 3-halo-4-methoxyphenyl, 3-halo-4-(C₁₋₃)alkylphenyl, 2-halo-5-trifluoromethylphenyl, 4-trifluoromethoxyphenyl, 2-, 3- or 4 (C₁₋₃)alkoxyphenyl, phenylcyclopropyl-, 2-, 3- or 4-(C₁₋₃)alkoxybenzyl, 3- or 4-cyanophenyl, 3- or 4-cyanobenzyl, 2- or 3-pyridyl, 2- or 3-furanyl, 2- or 3-thiophenyl, 3-halo-thiophen-2-yl, thieno[2,3-b]thiophenyl, 4,5-dihalo-isothiazole-3-yl, 3,6-dihalo-benzo[b]thiophen-2-yl, 5-halo-3-(C₁₋₃)alkyl-benzo[b]thiophen-2-yl, 5-halo-thiophen-2-yl, 3-bromo-5-chloro-thiophen-2-yl, and 3-halo-benzo[b]thiophen-2-yl, each further substituted or unsubstituted.

70. The compound according to claim 2, wherein L is a substituted or unsubstituted (C₁₋₆)alkyl.

71. The compound according to claim 2, wherein L is selected from the group consisting of methyl, ethyl, and propyl, each unsubstituted or substituted.

72. The compound according to claim 2, wherein L is a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl, each further substituted or unsubstituted.

73. The compound according to claim 1, wherein Lₐ is an unsubstituted or substituted (C₁₋₆)alkyl.

74. The compound according to claim 1, wherein Lₐ is selected from the group consisting of (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl, each further substituted or unsubstituted.

75. The compound according to claim 2, wherein L is selected from the group consisting of —CH₂—, —(CH₂)₂—, —(CH₂)₃—, —(CH₂)₄—, —(CH₂)₅—, and —(CH₂)₆—, each substituted or unsubstitute

76. The compound according to claim 2, wherein L is selected from the group consisting of arylene, (C₁₋₆)alkylarylene, aryl(C₁₋₆)alkylene, heteroarylene, and (C₁₋₆)alkylhetero(C₃₋₈)arylene, and hetero(C₃₋₈)aryl(C₁₋₆)alkylene, each substituted or unsubstituted.

77. The compound according to claim 3, wherein R₁₆ is a substituted or unsubstituted amino.

78. The compound according to claim 3, wherein R₁₆ is NH₂.

79. The compound according to claim 3, wherein R₁₆ is selected from the group consisting of 3-chloro-benzo[b]thiophene, 2,3-difluorophenyl, 4-methylthiophenyl, 2-ethoxyphenyl, 2-chloro-5-trifluoromethylphenyl, 1-naphthyl, 8-bromo-1-naphthyl, 3-fluoro-4-methoxyphenyl, 2-methyl-5-fluorophenyl, 4-chlorophenyl, 2,5-dichlorophenyl and 2-chlorothienyl, each further substituted or unsubstituted.

80. The compound according to claim 3, wherein R₁₆ is selected from the group consisting of phenyl, —CH═CH-phenyl, phenylcyclopropyl-, pyridinyl, quinolinyl, indolinyl, quinazolinyl, isothiazolyl, 1H-pyrazolyl, and thiophenyl, each substituted or unsubstituted.

81. The compound according to claim 3, wherein R₁₆ is selected from the group consisting of methyl, i-propyl, t-butyl, MeCO₂—, —CO₂H, cyclohexyl, hydroxy, methoxy, —NH₂, —NHMe, —NMe₂, —NEt₂, —NH(i-propyl), —N(i-propyl)₂, —NHBoc, phenyl, 2-methyl-, 2-ethyl- or 2-methoxy-phenyl, 3-methyl-, 3-ethyl-, or 3-hydroxyphenyl, and 2,5-dimethyl or 2,5-dimethoxyphenyl, each substituted or unsubstituted.

82. The compound according to claim 3, wherein R₁₆ is selected from the group consisting of pyrrolidin-1-yl, pyrrolidin-2-yl, pyrrolidin-3-yl, N-methyl or N-ethylpyrrolidin-2-yl, pyrrolidin-2-one-1-yl, piperazin-1-yl, morpholin-1-yl, 3-pyridyl, 1H-indol-1-yl, 1H-imidazol-4-yl, 1H-imidazol-1-yl, tetrahydrofuran-2-yl-methyl, azetidin-1-yl, azetidin-2-yl, azetidin-3-yl, piperidin-1-yl, 2-methylpiperidin-1-yl, piperidin-4-yl, piperidin-3-yl, piperidin-2-yl, and N-methylpiperidin-1-yl, each unsubstituted or further substituted.

83. The compound according to claim 3, wherein -L-R₁₆ together is selected from the group consisting of 3-dimethylamino-2,2-di(C₁₋₃)alkylpropanyl, 4-di(C₁₋₃)alkyl amino-pentan-2-yl, 4-di(C₁₋₃)alkylamino-1-cyclohexyl-pentanyl, 4-di(C₁₋₃)alkylamino-ethyl, and N-benzyl-piperidin-4-yl, each unsubstituted or further substituted.

84. The compound according to claim 1, wherein R₆, R₇, R₈, and R₉ are each independently selected from the group consisting of hydrogen, halo, a substituted or unsubstituted (C₁₋₆)alkyl, and cyano.

85. The compound according to claim 1, wherein R₆, R₇, R₈, R₉ are each independently hydrogen or are each selected from the group consisting of halo, halo(C₁₋₆)alkyl, amino, nitro, cyano, thio, (C₁₋₆)alkyl, cycloalkyl, heterocycloalkyl, aryl, heteroaryl, carbonyl(C₁₋₅)alkyl, thiocarbonyl (C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring.

86. The compound according to claim 1, wherein R₆, R₇, R₈, R₉ are each independently hydrogen or are each independently selected from the group consisting of (C₁₋₆)dialkylamino, (C₁₋₆)alkylsulfonyl, (C₁₋₆)alkylsulfonamido, sulfonamido(C₁₋₆)alkyl, sulfonamidoaryl, (C₁₋₆)alkylthio, carboxy(C₁₋₆)alkyl, carboxamido, aminocarbonyl, aryl and heteroaryl, each substituted or unsubstituted.

87. The compound according to claim 1, wherein R₆, R₇, R₈, R₉ are each independently hydrogen or are each independently selected from the group consisting of —C(O)NH₂, —SO₂NH₂, —COOH, (C₁₋₆)alkyl, (C₂₋₆)alkenyl, (C₂₋₆)alkenyl, (C₁₋₆)alkoxy, (C₁₋₆)alkoxyCO—, (C₁₋₆)alkylC)—, (C₁₋₆)alkylthio-, (C₁₋₆)alkylNH—, ((C₁₋₆)alkyl)₂N—, (C₁₋₆)alkylOCONH—, (C₁₋₆)alkylSO₂NH—, (C₁₋₆)alkylSONH—, and (C₁₋₆)alkylOCONH—, each unsubstituted or further substituted with the group selected from hydroxy, halogen, amino, cyano, nitro, (C₁₋₆)alkyl and halo(C₁₋₆)alkyl.

88. The compound according to claim 1, wherein R₆, R₇, R₈, R₉ are each independently selected from the group consisting of fluoro, chloro, bromo, iodo, cyano, and nitro.

89. The compound according to claim 1, wherein R₇ and R₈ are each independently selected from the group consisting of fluoro, chloro, bromo, iodo, cyano, and nitro.

90. The compound according to claim 1, wherein R₈ and R₉ are each H.

91. The compound according to claim 1, wherein R₇ and R₈ are each independently selected from the group consisting of halo, (C₁₋₆)alkyl, halo(C₁₋₆)alkyl, and (C₁₋₆)alkoxy.

92. A compound selected from the group consisting of:
(R)-{3-[1-(1-Benzyl-1H-benzoimidazol-2-yl)-2-methyl-propylamino]-propyl}-carbamic acid tert-butyl ester;
(R)—N-(3-amino-propyl)-N-[1-(1-benzyl-1H-benzoimidazol-2-yl)-2-methyl-propyl]-4-methyl-benzamide;
N-(3-Amino-propyl)-N-(1-benzyl-1H-benzoimidazol-2-ylmethyl)-4-methyl-benzamide;
(R)—N-(3-Amino-propyl)-N-[1-(1-benzyl-1H-benzoimidazol-2-yl)-ethyl]-4-methyl-benzamide;
(R)—N-(3-Amino-propyl)-N-[1-(1-benzyl-1H-benzoimidazol-2-yl)-propyl]-4-methyl-benzamide;
(R)—N-(3-Amino-propyl)-N-[1-(1-benzyl-1H-benzoimidazol-2-yl)-2-methyl-propyl]-4-methyl-benzamide;
(R)—N-(3-Amino-propyl)-N-[1-(1-benzyl-7-chloro-1H-benzoimidazol-2-yl)-2-methyl-propyl]-4-methyl-benzamide;
(R)—N-(3-Amino-propyl)-N-[1-(1-benzyl-6-chloro-1H-benzoimidazol-2-yl)-2-methyl-propyl]-4-methyl-benzamide;
(R)—N-(3-Amino-propyl)-N-[1-(1-benzyl-5-chloro-1H-benzoimidazol-2-yl)-2-methyl-propyl]-4-methyl-benzamide;
(R)—N-(3-Amino-propyl)-N-[1-(1-benzyl-5,6-dichloro-1H-benzoimidazol-2-yl)-2-methyl-propyl]-4-methyl-benzamide;
(R)—N-(3-Amino-propyl)-N-[1-(1-benzyl-7-methyl-1H-benzoimidazol-2-yl)-2-methyl-propyl]-4-methyl-benzamide;
(R)—N-(3-Amino-propyl)-N-[1-(1-benzyl-6-methyl-1H-benzoimidazol-2-yl)-2-methyl-propyl]-4-methyl-benzamide;
(R)—N-(3-Amino-propyl)-N-[1-(1-benzyl-5,6-dimethyl-1H-benzoimidazol-2-yl)-2-methyl-propyl]-4-methyl-benzamide;
(R)—N-(3-Amino-propyl)-N-[1-(1-benzyl-1H-benzoimidazol-2-yl)-2-methyl-propyl]-3-methyl-benzamide;
(R)—N-(3-Amino-propyl)-N-[1-(1-benzyl-1H-benzoimidazol-2-yl)-2-methyl-propyl]-2-methyl-benzamide;
(R)—N-(3-Amino-propyl)-N-[1-(1-benzyl-1H-benzoimidazol-2-yl)-2-methyl-propyl]-4-chloro-benzamide;
(R)—N-(3-Amino-propyl)-N-[1-(1-benzyl-1H-benzoimidazol-2-yl)-2-methyl-propyl]-3-chloro-benzamide;
(R)—N-(3-Amino-propyl)-N-[1-(1-benzyl-1H-benzoimidazol-2-yl)-2-methyl-propyl]-2-chloro-benzamide;
(R)—N-(3-Amino-propyl)-N-[1-(1-benzyl-1H-benzoimidazol-2-yl)-2-methyl-propyl]-4-methoxy-benzamide;
(R)—N-(3-Amino-propyl)-N-[1-(1-benzyl-1H-benzoimidazol-2-yl)-2-methyl-propyl]-3-methoxy-benzamide;
(R)—N-(3-Amino-propyl)-N-[1-(1-benzyl-1H-benzoimidazol-2-yl)-2-methyl-propyl]-2-methoxy-benzamide;
(R)—N-(3-Amino-propyl)-N-[1-(1-benzyl-1H-benzoimidazol-2-yl)-2-methyl-propyl]-4-cyano-benzamide;
(R)—N-(3-Amino-propyl)-N-[1-(1-benzyl-1H-benzoimidazol-2-yl)-2-methyl-propyl]-3-cyano-benzamide;
(R)—N-(3-Amino-propyl)-N-[1-(1-benzyl-1H-benzoimidazol-2-yl)-2-methyl-propyl]-nicotinamide;
(R)-Pyridine-2-carboxylic acid (3-amino-propyl)-[1-(1-benzyl-1H-benzoimidazol-2-yl)-2-methyl-propyl]-amide;
(R)-Furan-2-carboxylic acid (3-amino-propyl)-[1-(1-benzyl-1H-benzoimidazol-2-yl)-2-methyl-propyl]-amide;
(R)-Thiophene-2-carboxylic acid (3-amino-propyl)-[1-(1-benzyl-1H-benzo-imidazol-2-yl)-2-methyl-propyl]-amide;
(R)—N-(3-Amino-propyl)-N-[1-(1-benzyl-1H-benzoimidazol-2-yl)-2-methyl-propyl]-2-phenyl-acetamide;
(R)—N-(3-Amino-propyl)-N-[1-(1-benzyl-1H-benzoimidazol-2-yl)-2-methyl-propyl]-3-phenyl-propionamide;
(R)—N-(3-Amino-propyl)-N-[1-(1-benzyl-1H-benzoimidazol-2-yl)-2-methyl-propyl]-4-ethyl-benzamide;
(R)—N-(3-Amino-propyl)-4-methyl-N-[2-methyl-1-(1-methyl-1H-benzoimidazol-2-yl)-propyl]-benzamide;
(S)-N-(3-Amino-propyl)-N-[1-(1-benzyl-1H-benzoimidazol-2-yl)-2-methyl-propyl]-4-methyl-benzamide;
(R)—N-(3-aminopropyl)-N-(1-(1-benzyl-4-chloro-1H-benzo[d]imidazol-2-yl)-2-methylpropyl)-4-methylbenzamide;
(R)—N-(3-aminopropyl)-N-(1-(1-benzyl-1H-benzo[d]imidazol-2-yl)-2-methylpropyl)-5-ethylthiophene-3-carboxamide;
(R)—N-(3-aminopropyl)-N-(1-(1-benzyl-1H-benzo[d]imidazol-2-yl)-2-methylpropyl)-4-bromo-5-ethylthiophene-2-carboxamide;
(R)—N-(3-aminopropyl)-N-(1-(1-benzyl-1H-benzo[d]imidazol-2-yl)-2-methylpropyl)-4-bromo-5-propylthiophene-2-carboxamide;
(R)—N-(3-aminopropyl)-N-(1-(1-benzyl-1H-benzo[d]imidazol-2-yl)-2-methylpropyl)-5-methylthiophene-2-carboxamide;
(R)—N-(3-aminopropyl)-N-(1-(1-benzyl-1H-benzo[d]imidazol-2-yl)-2-methylpropyl)-5-isopropylthiophene-3-carboxamide;
(R)—N-(3-aminopropyl)-N-(1-(1-benzyl-1H-benzo[d]imidazol-2-yl)-2-methylpropyl)-5-(methylthio)thiophene-2-carboxamide;
(R)—N-(3-aminopropyl)-N-(1-(1-benzyl-1H-benzo[d]imidazol-2-yl)-2-methylpropyl)-4,5-dimethylthiophene-2-carboxamide;
(R)—N-(3-aminopropyl)-N-(1-(1-benzyl-1H-benzo[d]imidazol-2-yl)-2-methylpropyl)-4-bromothiophene-2-carboxamide;
(R)—N-(3-aminopropyl)-N-(1-(1-benzyl-1H-benzo[d]imidazol-2-yl)-2-methylpropyl)-4,5-dibromothiophene-2-carboxamide;
(R)—N-(3-aminopropyl)-N-(1-(1-benzyl-1H-benzo[d]imidazol-2-yl)-2-methylpropyl)thieno[2,3-b]thiophene-2-carboxamide;
(R)—N-(3-aminopropyl)-N-(1-(1-benzyl-4-methyl-1H-benzo[d]imidazol-2-yl)-2-methylpropyl)-4-methylbenzamide;
(R)—N-(3-aminopropyl)-N-(1-(1-benzyl-5-methyl-1H-benzo[d]imidazol-2-yl)-2-methylpropyl)-4-methylbenzamide;
(R)—N-(3-aminopropyl)-N-(1-(1-benzyl-4,5-dimethyl-1H-benzo[d]imidazol-2-yl)-2-methylpropyl)-4-methylbenzamide;
(R)—N-(3-aminopropyl)-N-(1-(1-benzyl-4,5-dichloro-1H-benzo[d]imidazol-2-yl)-2-methylpropyl)-4-methylbenzamide;
(R)—N-(3-aminopropyl)-N-(1-(1-benzyl-4-chloro-5-methyl-1H-benzo[d]imidazol-2-yl)-2-methylpropyl)-4-methylbenzamide;
(R)—N-(3-aminopropyl)-N-(1-(1-benzyl-5-chloro-4-methyl-1H-benzo[d]imidazol-2-yl)-2-methylpropyl)-4-methylbenzamide;
(R)—N-(3-aminopropyl)-N-(1-(1-benzyl-4-ethyl-1 H-benzo[d]imidazol-2-yl)-2-methylpropyl)-4-methylbenzamide;
(R)—N-(3-aminopropyl)-N-(1-(1-benzyl-5-ethyl-1H-benzo[d]imidazol-2-yl)-2-methylpropyl)-4-methylbenzamide;
(R)—N-(3-aminopropyl)-N-(1-(1-benzyl-4-ethyl-5-methyl-1H-benzo[d]imidazol-2-yl)-2-methylpropyl)-4-methylbenzamide;
(R)—N-(3-aminopropyl)-N-(1-(1-benzyl-5-chloro-4-ethyl-1H-benzo[d]imidazol-2-yl)-2-methylpropyl)-4-methylbenzamide;
(R)—N-(3-aminopropyl)-N-(1-(1-benzyl-5-ethyl-4-methyl-1H-benzo[d]imidazol-2-yl)-2-methylpropyl)-4-methylbenzamide;
(R)—N-(3-aminopropyl)-N-(1-(1-benzyl-4-chloro-5-ethyl-1H-benzo[d]imidazol-2-yl)-2-methylpropyl)-4-methylbenzamide;
(R)—N-(3-aminopropyl)-N-(1-(1-benzyl-4-cyano-1H-benzo[d]imidazol-2-yl)-2-methylpropyl)-4-methylbenzamide;
(R)—N-(2-aminoethyl)-N-(1-(1-benzyl-1H-benzo[d]imidazol-2-yl)-2-methylpropyl)-4-methylbenzamide;
(R)—N-(1-(1-benzyl-1H-benzo[d]imidazol-2-yl)-2-methylpropyl)-N-ethyl-4-methylbenzamide;
(R)—N-(1-(1-benzyl-1H-benzo[d]imidazol-2-yl)-2-methylpropyl)-4-methyl-N-propylbenzamide;
(R)—N-(1-(1-benzyl-1H-benzo[d]imidazol-2-yl)-2-methylpropyl)-N-(cyclopropylmethyl)-4-methylbenzamide;
(R)—N-(3-aminopropyl)-N-(1-(1-benzyl-1H-benzo[d]imidazol-2-yl)-2-methylpropyl)-6-methylnicotinamide;
(R)—N-(3-aminopropyl)-N-(1-(1-benzyl-1H-benzo[d]imidazol-2-yl)-2-methylpropyl)-6-chloronicotinamide;
(R)—N-(3-aminopropyl)-4-methyl-N-(2-methyl-1-(1-(thiophen-2-ylmethyl)-1H-benzo[d]imidazol-2-yl)propyl)benzamide;
(R)—N-(3-aminopropyl)-N-(1-(3-benzyl-3H-imidazo[4,5-b]pyridin-2-yl)-2-methylpropyl)-4-methylbenzamide;
(R)—N-(3-aminopropyl)-4-methyl-N-(2-methyl-1-(1-(pyridin-3-ylmethyl)-1H-benzo[d]imidazo1-2-yl)propyl)benzamide;
(R)—N-(3-aminopropyl)-N-(1-(9-benzyl-9H-purin-8-yl)-2-methylpropyl)-4-methylbenzamide;
(R)—N-(3-aminopropyl)-N-(1-(3-benzyl-7-chloro-3H-imidazo[4,5-c]pyridin-2-yl)-2-methylpropyl)-4-methylbenzamide; and
(R)—N-(1-(3-benzyl-7-chloro-3H-imidazo[4,5-e]pyridin-2-yl)-2-methylpropyl)-N-(cyclopropylmethyl)-4-methylbenzamide.

93. The compound according to claim 1, wherein the compound is in the form of a pharmaceutically acceptable salt.

94. The compound according to claim 1, wherein the compound is present in a mixture of stereoisomers.

95. The compounds according to claim 1, wherein the compound comprises a single stereoisomer.

96. A pharmaceutical composition comprising, as an active ingredient, a compound according to claim 1.

97. The pharmaceutical composition of claim 96 wherein the composition is a solid formulation adapted for oral administration.

98. The pharmaceutical composition of claim 97 wherein the composition is a tablet.

99. The pharmaceutical composition of claim 96 wherein the composition is a liquid formulation adapted for oral administration.

100. The pharmaceutical composition of claim 96 wherein the composition is a liquid formulation adapted for parenteral administration.

101. The pharmaceutical composition comprising a compound according to claim 1 wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, and intrathecally.

102. A kit comprising:
a compound according to claim 1; and
instructions which comprise one or more forms of information selected from the group consisting of indicating a disease state for which the compound is to be administered, storage information for the compound, dosing information and instructions regarding how to administer the compound.

103. The kit of claim 102 wherein the kit comprises the compound in a multiple dose form.

104. An article of manufacture comprising:
a compound according to claim 1; and
packaging materials.

105. The article of manufacture of claim 104 wherein the packaging material comprises a container for housing the compound.

106. The article of manufacture of claim 105 wherein the container comprises a label indicating one or more members of the group consisting of a disease state for which the compound is to be administered, storage information, dosing information and/or instructions regarding how to administer the composition.

107. The article of manufacture of claim 104 wherein the article of manufacture comprises the compound in a multiple dose form.

108. A method of treating cellular proliferative diseases comprising administering a compound of claim 1.

109. A method of treating a disorder associated with KSP kinesin activity comprising administering a compound of claim 1.

110. A method of inhibiting KSP kinesin comprising contacting KSP kinesin with a compound of claim 1.

111. The method of claim 109, wherein the disease or disorder is selected from the group consisting of cancer, hyperplasia, restenosis, cardiac hypertrophy, immune disorders and inflammation.

112. A method of inhibiting KSP kinesin comprising contacting KSP kinesin with a compound comprising the formula: 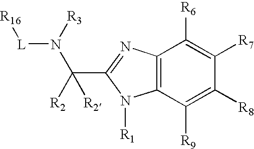
wherein:
R₁ is hydrogen, or is selected from the group consisting of (C₁₋₆)alkyl, (C₁₋₆)alkyloxy(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, (C₃₋₇)cycloalkyl, (C₁₋₆)alkyl(C₃₋₇)cycloalkyl, (C₃₋₇)cycloalkyl(C₁₋₆)alkylhetero(C₃₋₇)cycloalkyl, (C₁₋₆)alkylhetero(C₃₋₇)cycloalkyl, and hetero(C₃₋₇)cycloalkyl(C₁₋₆)alkyl each substituted or unsubstituted;
R₂ and R₂′ are each independently hydrogen or are each selected from the group consisting of (C₁₋₆)alkyl, (C₁₋₃)alkyloxy(C₁₋₃)alkyl, aryl, (C₁₋₆)alkylaryl, aryl(C₁₋₆)alkyl, heteroaryl, (C₁₋₆)alkylheteroaryl, heteroaryl(C_{1 -6})alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, and hetero(C₃₋₇)cycloalkyl(C₁₋₆)alkyl and the side chain of an amino acid, or R₂ and R₂′ are taken together to form a 3- to 7-membered carbocyclic or heterocyclic ring, each substituted or unsubstituted;
R₃ is selected from the group consisting of hydrogen, halo(C₁₋₆)alkyl, (C₁₋₁₅)alkyl, cycloalkyl, hetero(C₃₋₁₂)cycloalkyl, aryl, heteroaryl, carbonyl(C₁₋₅)alkyl, thiocarbonyl (C₁₋₅)alkyl, sulfonyl(C₁₋₃)alkyl, sulfinyl(C₁₋₃)alkyl, imino(C₁₋₃)alkyl, carbonyl group, imino group, sulfonyl group, sulfinyl group and sulfonamido, each substituted or unsubstituted, or R₃ and R₂ or R₂′ are taken together to form a 4- to 7-membered substituted or unsubstituted ring;
R₆, R₇, R₈, and R₉ are each independently hydrogen or are each selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₅)alkyl, thiocarbonyl(C₁₋₅)alkyl, sulfonyl(C₁₋₃)alkyl, sulfinyl(C₁₋₃)alkyl, imino(C₁₋₃)alkyl, hydroxy, (C₁₋₆)alkoxy, carbonyl group, imino group, sulfonyl group, sulfinyl group and sulfonamido, each substituted or unsubstituted, or two of R₆, R₇, R₈, and R₉ are taken together to form a substituted or unsubstituted 4, 5, 6 or 7 membered carbocyclic or heterocyclic ring;
R₁₆ is selected from the group consisting of halo, halo(C₁₋₆)alkyl, amino, nitro, cyano, thio, (C₁₋₆)alkyl, cycloalkyl, hetero(C₃₋₁₂)cycloalkyl, aryl, heteroaryl, carbonyl(C₁₋₅)alkyl, thiocarbonyl(C₁₋₅)alkyl, sulfonyl(C₁₋₃)alkyl, sulfinyl imino(C₁₋₃)alkyl, hydroxy, (C₁₋₆)alkoxy, aryloxy, heteroaryloxy, carbonyl group, imino group, sulfonyl group, sulfinyl group and sulfonamido, each substituted or unsubstituted, and a substituted or unsubstituted 4, 5, 6 or 7 membered ring; and
L is a bond, or a linker comprising a backbone chain of 1 to 10 atoms comprising C, N, O, or S and may be optionally substituted with halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.
